SEQUENCE LISTING (27 sequences)

SEQ ID NO: 1 - protein, 290 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 2 - protein, 417 aa, Homo sapiens
MARAMAAAWPLLLVALLVLSWPPPGTGDVVVQAPTQVPGFLGDSVTLPCYLQVPNMEVTHVSQLTWARHGESGSMAVFHQTQGPSYSESKRLEFVAARLGAELRNASLRMFGLRVEDEGNYTCLFVTFPQGSRSVDIWLRVLAKPQNTAEVQKVQLTGEPVPMARCVSTGGRPPAQITWHSDLGGMPNTSQVPGFLSGTVTVTSLWILVPSSQVDGKNVTCKVEHESFEKPQLLTVNLTVYYPPEVSISGYDNNWYLGQNEATLTCDARSNPEPTGYNWSTTMGPLPPFAVAQGAQLLIRPVDKPINTTLICNVTNALGARQAELTVQVKEGPPSEHSGISRNAIIFLVLGILVFLILLGIGIYFYWSKCSREVLWHCHLCPSSTEHASASANGHVSYSAVSRENSSSQDPQTEGTR

SEQ ID NO: 3 - protein, 538 aa, Homo sapiens
MARAAALLPSRSPPTPLLWPLLLLLLLETGAQDVRVQVLPEVRGQLGGTVELPCHLLPPVPGLYISLVTWQRPDAPANHQNVAAFHPKMGPSFPSPKPGSERLSFVSAKQSTGQDTEAELQDATLALHGLTVEDEGNYTCEFATFPKGSVRGMTWLRVIAKPKNQAEAQKVTFSQDPTTVALCISKEGRPPARISWLSSLDWEAKETQVSGTLAGTVTVTSRFTLVPSGRADGVTVTCKVEHESFEEPALIPVTLSVRYPPEVSISGYDDNWYLGRTDATLSCDVRSNPEPTGYDWSTTSGTFPTSAVAQGSQLVIHAVDSLFNTTFVCTVTNAVGMGRAEQVIFVRETPNTAGAGATGGIIGGIIAAIIATAVAATGILICRQQRKEQTLQGAEEDEDLEGPPSYKPPTPKAKLEAQEMPSQLFTLGASEHSPLKTPYFDAGASCTEQEMPRYHELPTLEERSGPLHPGATSLGSPIPVPPGPPAVEDVSLDLEDEEGEEEEEYLDKINPIYDALSYSSPSDSYQGKGFVMSRAMYV

SEQ ID NO: 4 - protein, 312 aa, Homo sapiens
MAKVFSFILVTTALTMGREISALEDCAQEQMRLRAQVRLLETRVKQQQVKIKQLLQENEVQFLDKGDENTVIDLGSKRQYADCSEIFNDGYKLSGFYKIKPLQSPAEFSVYCDMSDGGGWTVIQRRSDGSENFNRGWKDYENGFGNFVQKHGEYWLGNKNLHFLTTQEDYTLKIDLADFEKNSRYAQYKNFKVGDEKNFYELNIGEYSGTAGDSLAGNFHPEVQWWASHQRMKFSTWDRDHDNYEGNCAEEDQSGWWFNRCHSANLNGVYYSGPYTAKTDNGIVWYTWHGWWYSLKSVVMKIRPNDFIPNVI

SEQ ID NO: 5 - protein, 355 aa, Homo sapiens
MAFSGSQAPYLSPAVPFSGTIQGGLQDGLQITVNGTVLSSSGTRFAVNFQTGFSGNDIAFHFNPRFEDGGYVVCNTRQNGSWGPEERKTHMPFQKGMPFDLCFLVQSSDFKVMVNGILFVQYFHRVPFHRVDTISVNGSVQLSYISFQNPRTVPVQPAFSTVPFSQPVCFPPRPRGRRQKPPGVWPANPAPITQTVIHTVQSAPGQMFSTPAIPPMMYPHPAYPMPFITTILGGLYPSKSILLSGTVLPSAQRFHINLCSGNHIAFHLNPRFDENAVVRNTQIDNSWGSEERSLPRKMPFVRGQSFSVWILCEAHCLKVAVDGQHLFEYYHRLRNLPTINRLEVGGDIQLTHVQT

SEQ ID NO: 6 - protein, 323 aa, Homo sapiens
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 7 - protein, 534 aa, Homo sapiens
MLRRRGSPGMGVHVGAALGALWFCLTGALEVQVPEDPVVALVGTDATLCCSFSPEPGFSLAQLNLIWQLTDTKQLVHSFAEGQDQGSAYANRTALFPDLLAQGNASLRLQRVRVADEGSFTCFVSIRDFGSAAVSLQVAAPYSKPSMTLEPNKDLRPGDTVTITCSSYQGYPEAEVFWQDGQGVPLTGNVTTSQMANEQGLFDVHSILRVVLGANGTYSCLVRNPVLQQDAHSSVTITPQRSPTGAVEVQVPEDPVVALVGTDATLRCSFSPEPGFSLAQLNLIWQLTDTKQLVHSFTEGRDQGSAYANRTALFPDLLAQGNASLRLQRVRVADEGSFTCFVSIRDFGSAAVSLQVAAPYSKPSMTLEPNKDLRPGDTVTITCSSYRGYPEAEVFWQDGQGVPLTGNVTTSQMANEQGLFDVHSVLRVVLGANGTYSCLVRNPVLQQDAHGSVTITGQPMTFPPEALWVTVGLSVCLIALLVALAFVCWRKIKQSCEEENAGAEDQDGEGEGSKTALQPLKHSDSKEDDGQEIA

SEQ ID NO: 8 - protein, 282 aa, Homo sapiens
MASLGQILFWSIISIIIILAGAIALIIGFGISGRHSITVTTVASAGNIGEDGILSCTFEPDIKLSDIVIQWLKEGVLGLVHEFKEGKDELSEQDEMFRGRTAVFADQVIVGNASLRLKNVQLTDAGTYKCYIITSKGKGNANLEYKTGAFSMPEVNVDYNASSETLRCEAPRWFPQPTVVWASQVDQGANFSEVSNTSFELNSENVTMKVVSVLYNVTINNTYSCMIENDIAKATGDIKVTESEIKRRSHLQLLNSKASLCVSSFFAISWALLPLSPYLMLK

SEQ ID NO: 9 - protein, 486 aa, Artificial Sequence
  Synthetic Construct
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 10 - protein, 487 aa, Artificial Sequence
  Synthetic Construct
MALPVTALLLPLALLLHAARPAVQLVESGGGLVQAGDSLRLTCTASGRAFSTYFMAWFRQAPGKEREFVAGIAWSGGSTAYADSVKGRFTISRDNAKNTVYLQMNSLKSEDTAVYYCASRGIEVEEFGAWGQGTQVTVSSGGGGSQVQLEESGGGSVQAGGSLRLSCAYTYSTYSNYYMGWFREAPGKARTSVAIISSDTTITYKDAVKGRFTISKDNAKNTLYLQMNSLKPEDSAMYRCAAWTSDWSVAYWGQGTQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 11 - protein, 22 aa, Artificial Sequence
  Synthetic Construct
GSGATNFSLLKQAGDVEENPGP

SEQ ID NO: 12 - protein, 798 aa, Artificial Sequence
  Synthetic Construct
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 13 - protein, 925 aa, Homo sapiens
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMARAMAAAWPLLLVALLVLSWPPPGTGDVVVQAPTQVPGFLGDSVTLPCYLQVPNMEVTHVSQLTWARHGESGSMAVFHQTQGPSYSESKRLEFVAARLGAELRNASLRMFGLRVEDEGNYTCLFVTFPQGSRSVDIWLRVLAKPQNTAEVQKVQLTGEPVPMARCVSTGGRPPAQITWHSDLGGMPNTSQVPGFLSGTVTVTSLWILVPSSQVDGKNVTCKVEHESFEKPQLLTVNLTVYYPPEVSISGYDNNWYLGQNEATLTCDARSNPEPTGYNWSTTMGPLPPFAVAQGAQLLIRPVDKPINTTLICNVTNALGARQAELTVQVKEGPPSEHSGISRNAIIFLVLGILVFLILLGIGIYFYWSKCSREVLWHCHLCPSSTEHASASANGHVSYSAVSRENSSSQDPQTEGTR

SEQ ID NO: 14 - protein, 1046 aa, Homo sapiens
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMARAAALLPSRSPPTPLLWPLLLLLLLETGAQDVRVQVLPEVRGQLGGTVELPCHLLPPVPGLYISLVTWQRPDAPANHQNVAAFHPKMGPSFPSPKPGSERLSFVSAKQSTGQDTEAELQDATLALHGLTVEDEGNYTCEFATFPKGSVRGMTWLRVIAKPKNQAEAQKVTFSQDPTTVALCISKEGRPPARISWLSSLDWEAKETQVSGTLAGTVTVTSRFTLVPSGRADGVTVTCKVEHESFEEPALIPVTLSVRYPPEVSISGYDDNWYLGRTDATLSCDVRSNPEPTGYDWSTTSGTFPTSAVAQGSQLVIHAVDSLFNTTFVCTVTNAVGMGRAEQVIFVRETPNTAGAGATGGIIGGIIAAIIATAVAATGILICRQQRKEQTLQGAEEDEDLEGPPSYKPPTPKAKLEAQEMPSQLFTLGASEHSPLKTPYFDAGASCTEQEMPRYHELPTLEERSGPLHPGATSLGSPIPVPPGPPAVEDVSLDLEDEEGEEEEEYLDKINPIYDALSYSSPSDSYQGKGFVMSRAMYV

SEQ ID NO: 15 - protein, 820 aa, Homo sapiens
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMAKVFSFILVTTALTMGREISALEDCAQEQMRLRAQVRLLETRVKQQQVKIKQLLQENEVQFLDKGDENTVIDLGSKRQYADCSEIFNDGYKLSGFYKIKPLQSPAEFSVYCDMSDGGGWTVIQRRSDGSENFNRGWKDYENGFGNFVQKHGEYWLGNKNLHFLTTQEDYTLKIDLADFEKNSRYAQYKNFKVGDEKNFYELNIGEYSGTAGDSLAGNFHPEVQWWASHQRMKFSTWDRDHDNYEGNCAEEDQSGWWFNRCHSANLNGVYYSGPYTAKTDNGIVWYTWHGWWYSLKSVVMKIRPNDFIPNVI

SEQ ID NO: 16 - protein, 863 aa, Homo sapiens
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMAFSGSQAPYLSPAVPFSGTIQGGLQDGLQITVNGTVLSSSGTRFAVNFQTGFSGNDIAFHFNPRFEDGGYVVCNTRQNGSWGPEERKTHMPFQKGMPFDLCFLVQSSDFKVMVNGILFVQYFHRVPFHRVDTISVNGSVQLSYISFQNPRTVPVQPAFSTVPFSQPVCFPPRPRGRRQKPPGVWPANPAPITQTVIHTVQSAPGQMFSTPAIPPMMYPHPAYPMPFITTILGGLYPSKSILLSGTVLPSAQRFHINLCSGNHIAFHLNPRFDENAVVRNTQIDNSWGSEERSLPRKMPFVRGQSFSVWILCEAHCLKVAVDGQHLFEYYHRLRNLPTINRLEVGGDIQLTHVQT

SEQ ID NO: 17 - protein, 831 aa, Homo sapiens
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 18 - protein, 1042 aa, Homo sapiens
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMLRRRGSPGMGVHVGAALGALWFCLTGALEVQVPEDPVVALVGTDATLCCSFSPEPGFSLAQLNLIWQLTDTKQLVHSFAEGQDQGSAYANRTALFPDLLAQGNASLRLQRVRVADEGSFTCFVSIRDFGSAAVSLQVAAPYSKPSMTLEPNKDLRPGDTVTITCSSYQGYPEAEVFWQDGQGVPLTGNVTTSQMANEQGLFDVHSILRVVLGANGTYSCLVRNPVLQQDAHSSVTITPQRSPTGAVEVQVPEDPVVALVGTDATLRCSFSPEPGFSLAQLNLIWQLTDTKQLVHSFTEGRDQGSAYANRTALFPDLLAQGNASLRLQRVRVADEGSFTCFVSIRDFGSAAVSLQVAAPYSKPSMTLEPNKDLRPGDTVTITCSSYRGYPEAEVFWQDGQGVPLTGNVTTSQMANEQGLFDVHSVLRVVLGANGTYSCLVRNPVLQQDAHGSVTITGQPMTFPPEALWVTVGLSVCLIALLVALAFVCWRKIKQSCEEENAGAEDQDGEGEGSKTALQPLKHSDSKEDDGQEIA

SEQ ID NO: 19 - protein, 790 aa, Homo sapiens
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMASLGQILFWSIISIIIILAGAIALIIGFGISGRHSITVTTVASAGNIGEDGILSCTFEPDIKLSDIVIQWLKEGVLGLVHEFKEGKDELSEQDEMFRGRTAVFADQVIVGNASLRLKNVQLTDAGTYKCYIITSKGKGNANLEYKTGAFSMPEVNVDYNASSETLRCEAPRWFPQPTVVWASQVDQGANFSEVSNTSFELNSENVTMKVVSVLYNVTINNTYSCMIENDIAKATGDIKVTESEIKRRSHLQLLNSKASLCVSSFFAISWALLPLSPYLMLK

SEQ ID NO: 20 - protein, 799 aa, Homo sapiens
MALPVTALLLPLALLLHAARPAVQLVESGGGLVQAGDSLRLTCTASGRAFSTYFMAWFRQAPGKEREFVAGIAWSGGSTAYADSVKGRFTISRDNAKNTVYLQMNSLKSEDTAVYYCASRGIEVEEFGAWGQGTQVTVSSGGGGSQVQLEESGGGSVQAGGSLRLSCAYTYSTYSNYYMGWFREAPGKARTSVAIISSDTTITYKDAVKGRFTISKDNAKNTLYLQMNSLKPEDSAMYRCAAWTSDWSVAYWGQGTQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 21 - protein, 926 aa, Homo sapiens
MALPVTALLLPLALLLHAARPAVQLVESGGGLVQAGDSLRLTCTASGRAFSTYFMAWFRQAPGKEREFVAGIAWSGGSTAYADSVKGRFTISRDNAKNTVYLQMNSLKSEDTAVYYCASRGIEVEEFGAWGQGTQVTVSSGGGGSQVQLEESGGGSVQAGGSLRLSCAYTYSTYSNYYMGWFREAPGKARTSVAIISSDTTITYKDAVKGRFTISKDNAKNTLYLQMNSLKPEDSAMYRCAAWTSDWSVAYWGQGTQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMARAMAAAWPLLLVALLVLSWPPPGTGDVVVQAPTQVPGFLGDSVTLPCYLQVPNMEVTHVSQLTWARHGESGSMAVFHQTQGPSYSESKRLEFVAARLGAELRNASLRMFGLRVEDEGNYTCLFVTFPQGSRSVDIWLRVLAKPQNTAEVQKVQLTGEPVPMARCVSTGGRPPAQITWHSDLGGMPNTSQVPGFLSGTVTVTSLWILVPSSQVDGKNVTCKVEHESFEKPQLLTVNLTVYYPPEVSISGYDNNWYLGQNEATLTCDARSNPEPTGYNWSTTMGPLPPFAVAQGAQLLIRPVDKPINTTLICNVTNALGARQAELTVQVKEGPPSEHSGISRNAIIFLVLGILVFLILLGIGIYFYWSKCSREVLWHCHLCPSSTEHASASANGHVSYSAVSRENSSSQDPQTEGTR

SEQ ID NO: 22 - protein, 1047 aa, Homo sapiens
MALPVTALLLPLALLLHAARPAVQLVESGGGLVQAGDSLRLTCTASGRAFSTYFMAWFRQAPGKEREFVAGIAWSGGSTAYADSVKGRFTISRDNAKNTVYLQMNSLKSEDTAVYYCASRGIEVEEFGAWGQGTQVTVSSGGGGSQVQLEESGGGSVQAGGSLRLSCAYTYSTYSNYYMGWFREAPGKARTSVAIISSDTTITYKDAVKGRFTISKDNAKNTLYLQMNSLKPEDSAMYRCAAWTSDWSVAYWGQGTQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMARAAALLPSRSPPTPLLWPLLLLLLLETGAQDVRVQVLPEVRGQLGGTVELPCHLLPPVPGLYISLVTWQRPDAPANHQNVAAFHPKMGPSFPSPKPGSERLSFVSAKQSTGQDTEAELQDATLALHGLTVEDEGNYTCEFATFPKGSVRGMTWLRVIAKPKNQAEAQKVTFSQDPTTVALCISKEGRPPARISWLSSLDWEAKETQVSGTLAGTVTVTSRFTLVPSGRADGVTVTCKVEHESFEEPALIPVTLSVRYPPEVSISGYDDNWYLGRTDATLSCDVRSNPEPTGYDWSTTSGTFPTSAVAQGSQLVIHAVDSLFNTTFVCTVTNAVGMGRAEQVIFVRETPNTAGAGATGGIIGGIIAAIIATAVAATGILICRQQRKEQTLQGAEEDEDLEGPPSYKPPTPKAKLEAQEMPSQLFTLGASEHSPLKTPYFDAGASCTEQEMPRYHELPTLEERSGPLHPGATSLGSPIPVPPGPPAVEDVSLDLEDEEGEEEEEYLDKINPIYDALSYSSPSDSYQGKGFVMSRAMYV

SEQ ID NO: 23 - protein, 821 aa, Homo sapiens
MALPVTALLLPLALLLHAARPAVQLVESGGGLVQAGDSLRLTCTASGRAFSTYFMAWFRQAPGKEREFVAGIAWSGGSTAYADSVKGRFTISRDNAKNTVYLQMNSLKSEDTAVYYCASRGIEVEEFGAWGQGTQVTVSSGGGGSQVQLEESGGGSVQAGGSLRLSCAYTYSTYSNYYMGWFREAPGKARTSVAIISSDTTITYKDAVKGRFTISKDNAKNTLYLQMNSLKPEDSAMYRCAAWTSDWSVAYWGQGTQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMAKVFSFILVTTALTMGREISALEDCAQEQMRLRAQVRLLETRVKQQQVKIKQLLQENEVQFLDKGDENTVIDLGSKRQYADCSEIFNDGYKLSGFYKIKPLQSPAEFSVYCDMSDGGGWTVIQRRSDGSENFNRGWKDYENGFGNFVQKHGEYWLGNKNLHFLTTQEDYTLKIDLADFEKNSRYAQYKNFKVGDEKNFYELNIGEYSGTAGDSLAGNFHPEVQWWASHQRMKFSTWDRDHDNYEGNCAEEDQSGWWFNRCHSANLNGVYYSGPYTAKTDNGIVWYTWHGWWYSLKSVVMKIRPNDFIPNVI

SEQ ID NO: 24 - protein, 864 aa, Homo sapiens
MALPVTALLLPLALLLHAARPAVQLVESGGGLVQAGDSLRLTCTASGRAFSTYFMAWFRQAPGKEREFVAGIAWSGGSTAYADSVKGRFTISRDNAKNTVYLQMNSLKSEDTAVYYCASRGIEVEEFGAWGQGTQVTVSSGGGGSQVQLEESGGGSVQAGGSLRLSCAYTYSTYSNYYMGWFREAPGKARTSVAIISSDTTITYKDAVKGRFTISKDNAKNTLYLQMNSLKPEDSAMYRCAAWTSDWSVAYWGQGTQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMAFSGSQAPYLSPAVPFSGTIQGGLQDGLQITVNGTVLSSSGTRFAVNFQTGFSGNDIAFHFNPRFEDGGYVVCNTRQNGSWGPEERKTHMPFQKGMPFDLCFLVQSSDFKVMVNGILFVQYFHRVPFHRVDTISVNGSVQLSYISFQNPRTVPVQPAFSTVPFSQPVCFPPRPRGRRQKPPGVWPANPAPITQTVIHTVQSAPGQMFSTPAIPPMMYPHPAYPMPFITTILGGLYPSKSILLSGTVLPSAQRFHINLCSGNHIAFHLNPRFDENAVVRNTQIDNSWGSEERSLPRKMPFVRGQSFSVWILCEAHCLKVAVDGQHLFEYYHRLRNLPTINRLEVGGDIQLTHVQT

SEQ ID NO: 25 - protein, 832 aa, Homo sapiens
MALPVTALLLPLALLLHAARPAVQLVESGGGLVQAGDSLRLTCTASGRAFSTYFMAWFRQAPGKEREFVAGIAWSGGSTAYADSVKGRFTISRDNAKNTVYLQMNSLKSEDTAVYYCASRGIEVEEFGAWGQGTQVTVSSGGGGSQVQLEESGGGSVQAGGSLRLSCAYTYSTYSNYYMGWFREAPGKARTSVAIISSDTTITYKDAVKGRFTISKDNAKNTLYLQMNSLKPEDSAMYRCAAWTSDWSVAYWGQGTQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 26 - protein, 1043 aa, Homo sapiens
MALPVTALLLPLALLLHAARPAVQLVESGGGLVQAGDSLRLTCTASGRAFSTYFMAWFRQAPGKEREFVAGIAWSGGSTAYADSVKGRFTISRDNAKNTVYLQMNSLKSEDTAVYYCASRGIEVEEFGAWGQGTQVTVSSGGGGSQVQLEESGGGSVQAGGSLRLSCAYTYSTYSNYYMGWFREAPGKARTSVAIISSDTTITYKDAVKGRFTISKDNAKNTLYLQMNSLKPEDSAMYRCAAWTSDWSVAYWGQGTQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMLRRRGSPGMGVHVGAALGALWFCLTGALEVQVPEDPVVALVGTDATLCCSFSPEPGFSLAQLNLIWQLTDTKQLVHSFAEGQDQGSAYANRTALFPDLLAQGNASLRLQRVRVADEGSFTCFVSIRDFGSAAVSLQVAAPYSKPSMTLEPNKDLRPGDTVTITCSSYQGYPEAEVFWQDGQGVPLTGNVTTSQMANEQGLFDVHSILRVVLGANGTYSCLVRNPVLQQDAHSSVTITPQRSPTGAVEVQVPEDPVVALVGTDATLRCSFSPEPGFSLAQLNLIWQLTDTKQLVHSFTEGRDQGSAYANRTALFPDLLAQGNASLRLQRVRVADEGSFTCFVSIRDFGSAAVSLQVAAPYSKPSMTLEPNKDLRPGDTVTITCSSYRGYPEAEVFWQDGQGVPLTGNVTTSQMANEQGLFDVHSVLRVVLGANGTYSCLVRNPVLQQDAHGSVTITGQPMTFPPEALWVTVGLSVCLIALLVALAFVCWRKIKQSCEEENAGAEDQDGEGEGSKTALQPLKHSDSKEDDGQEIA

SEQ ID NO: 27 - protein, 791 aa, Homo sapiens
MALPVTALLLPLALLLHAARPAVQLVESGGGLVQAGDSLRLTCTASGRAFSTYFMAWFRQAPGKEREFVAGIAWSGGSTAYADSVKGRFTISRDNAKNTVYLQMNSLKSEDTAVYYCASRGIEVEEFGAWGQGTQVTVSSGGGGSQVQLEESGGGSVQAGGSLRLSCAYTYSTYSNYYMGWFREAPGKARTSVAIISSDTTITYKDAVKGRFTISKDNAKNTLYLQMNSLKPEDSAMYRCAAWTSDWSVAYWGQGTQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMASLGQILFWSIISIIIILAGAIALIIGFGISGRHSITVTTVASAGNIGEDGILSCTFEPDIKLSDIVIQWLKEGVLGLVHEFKEGKDELSEQDEMFRGRTAVFADQVIVGNASLRLKNVQLTDAGTYKCYIITSKGKGNANLEYKTGAFSMPEVNVDYNASSETLRCEAPRWFPQPTVVWASQVDQGANFSEVSNTSFELNSENVTMKVVSVLYNVTINNTYSCMIENDIAKATGDIKVTESEIKRRSHLQLLNSKASLCVSSFFAISWALLPLSPYLMLK

(19) United States
(12) Patent Application Publication
(10) Pub. No.: US 2023/0149461 A1
(43) Pub. Date: May 18, 2023

(54) COMPOSITIONS AND METHODS FOR REDUCING GRAFT REJECTION IN ALLOGENEIC CELL THERAPY

(71) Applicant: Nanjing Legend Biotech Co., Ltd., Nanjing, Jiangsu (CN)

(72) Inventors: Zhongyuan TU, Nanjing, Jiangsu (CN); Jie LI, Nanjing, Jiangsu (CN); Yafeng ZHANG, Nanjing, Jiangsu (CN); Shu WU, Nanjing, Jiangsu (CN)

(21) Appl. No.: 17/916,370

(22) PCT Filed: Apr. 1, 2021
(86) PCT No.: PCT/CN2021/084956

§ 371 Date: Sep. 30, 2022

(30) Foreign Application Priority Data
Apr. 1, 2020 (CN) PCT/CN2020/082759

(51) Int. Cl. A61K 35/17; C12N 5/0783; A61K 38/17; A61K 35/15; A61P 37/06; A61P 35/00

(52) U.S. Cl. CPC A61K 35/17; C12N 5/0636; A61K 38/1774; A61K 35/15; A61P 37/06; A61P 35/00; C12N 2501/2302

(57) ABSTRACT

Provided are modified therapeutic cells that overexpress an immune checkpoint ligand such as PD-L1, CD155, CD112, FGL1, galectin-9, CD47, B7H3 and B7H4, while expressing one or more Major Histocompatibility Complex (MHC) molecules. Also provided are methods of treatment using the modified therapeutic cells.

### CROSS REFERENCE TO RELATED APPLICATIONS

This application claims priority benefits of International Patent Applications No PCT/CN2020/082759 filed on Apr. 1, 2020, the contents of which are incorporated herein by reference in their entirety.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 761422002341.txt, date recorded: Mar. 25, 2021, size: 149 KB).

### FIELD OF THE PRESENT APPLICATION

The present application generally relates to immunotherapy, and more specifically to the compositions and methods for reducing undesired immune response in adoptive cell therapy.

### BACKGROUND OF THE PRESENT APPLICATION

Adoptive cell therapy or adoptive cell transfer (ACT) is becoming an ever more important treatment paradigm, particularly in the treatment of cancer. ACT refers to the transfer of therapeutic cells, most typically immune cells, into a patient. These cells may have originated from the patient (i.e., autologous therapy) or from another individual of the same species (i.e., allogeneic therapy). The goal of ACT is to improve functions and characteristics of the immune system in the patient. Specially, in cancer immunotherapy, the goal of ACT is to trigger an immune response against the cancer. Although T cells are most often used in ACT, other immune cell types such as NK cells, lymphocytes (e.g., tumor-infiltrating lymphocytes or TILs), dendritic cells and myeloid cells have also been applied.

Ideally, the therapeutic cells that are infused to a patient receiving an ACT (or reinfused in case of autologous therapy) will expand and persist in the patient. To improve ACT efficacy, lymphodepletion is often used as neoadjuvant therapy, which ablates competing immune cells that may repopulate the immune cell space. This is especially important for allogeneic therapy because an immune response may be raised against infused non-self cells, both by the adaptive and the innate immune systems. Sometimes, myeloablation, which applies high-dose chemotherapy to kill cells in the bone marrow, is also used. However, lymphodepletion or myeloablation are quite drastic measures that often result in severe side effects because of their effects on the immune system. Accordingly, it would be advantageous to prevent or reduce undesired immune response against infused ACT cells, which may increase the persistence of the ACT cells in vivo and enhance the benefits of the therapy. Ideally, such strategy would lead to reduced need for lymphodepletion or myeloablation and the associated side effects.

The immune system has developed elaborate and effective mechanisms against foreign agents. Undesired immune response presents the most formidable barriers against successful ACT, especially allogeneic ACT, including allogeneic Chimeric Antigen Receptor (CAR) T cells. One barrier is graft-versus-host disease (GvHD), in which the donor T cells recognize the patient's cells as foreign, resulting in attack of the patient's healthy tissues. Another barrier is Host-versus-grail disease (HvGD), in which T cells in the patient (i.e., host) recognize the donor cells as foreign, resulting in attack of the donor cells and graft rejection. GvHD is mediated by T cell receptors (TCR) on the surface of donor T cells, which recognize human leukocyte antigen (HLA) on the host cells as foreign and initiate the attack by the donor T cells. HvGD is mediated by TCRs on the surface of host T cells, which recognize HLA on the donor cells as foreign and initiate the attack by the host T cells.

To mitigate GvHD, genome-editing technologies have been used to knockout TCR genes from the genome of donor CAR-T cells. Many companies also knockout beta-2 microglobulin (B2M) in donor CAR-T cells to reduce graft rejection caused by host T cells. Recently developed genome-editing tools such as transcription activator-like effector nucleases (TALENs), zinc finger nucleases and CRISPR-Cas9 systems are effective and easy to easy. However, genome editing in clinically relevant human somatic cells remains a challenge because of immunogenicity of the genome-edited cells. For example, B2M knockout increases the risk of graft rejection mediated by Natural Killer (NK) cells. To date, allogeneic αβ T cell therapy products have yet to achieve promising clinical results (Sommer C et al., Mol Ther. (2019) 27(6):1126-1138; MacLeod D T et al., Mol Ther. (2017) 25(4):949-96; Sheridan C et al., Nat Biotechnol (2018) 36, 375-377; Qasim W. Am J Hematol. 2019; 94: S50-S54). Accordingly, there exists a need for therapeutic cells suitable for allogeneic transplantation with reduced risk of undesired immune response.

The disclosures of all publications, patents, patent applications and published patent applications referred to herein are hereby incorporated herein by reference in their entirety.

### BRIEF SUMMARY OF THE INVENTION

The present application provides modified therapeutic cells that are suitable for adoptive cell therapy with reduced undesired immune response (e.g., graft rejection against the therapeutic cells) and methods of use thereof.

One aspect of the present application provides a modified therapeutic cell comprising a first heterologous nucleic acid sequence encoding an immune checkpoint ligand (ICL), wherein the therapeutic cell expresses a Major Histocompatibility Complex (MHC) molecule.

One aspect of the present application provides a method of reducing graft rejection of allogeneic therapeutic cells in an individual in need thereof, comprising administering to the individual an effective amount of the allogeneic therapeutic cells, wherein the allogeneic therapeutic cells comprise a first heterologous nucleic acid sequence encoding an immune checkpoint ligand (ICL), and wherein the therapeutic cells express an MHC molecule.

In some embodiments according to any one of the modified therapeutic cells or methods described above, the therapeutic cell expresses an MHC class I molecule. In some embodiments, the therapeutic cell expresses an MHC class II molecule. In some embodiments, the β2-microglobulin (B2M) gene of the therapeutic cell is not genetically modified. In some embodiments, the MHC genes of the therapeutic cell are not genetically modified. In some embodiments, the therapeutic cells do not elicit proliferation or killing by NK cells.

In some embodiments according to any one of the modified therapeutic cells or methods described above, the ICL is selected from the group consisting of PD-L1, CD155, CD112, FGL1, galectin-9, CD47, B7H3 and B7H4. In some embodiments, the ICL is PD-LL. In some embodiments, the ICL is CD155. In some embodiments, the ICL is CD112. In some embodiments, the ICL is FGL1. In some embodiments, the ICL is galectin-9. In some embodiments, the ICL is CD47. In some embodiments, the ICL is B7H3. In some embodiments, the ICL is B7H4. In some embodiments, the ICL comprises an amino acid sequence having at least about 85% (e.g., at least about any one of 90%, 91%, 92%, 93%, 94%, 95%, 96%, 97%, 98%, 99%, or more) sequence identity to an amino acid sequence selected from the group consisting of SEQ ID NOs: 1-8. In some embodiments, the ICL comprises an amino acid sequence selected from the group consisting of SEQ ID NOs: 1-8.

In some embodiments according to any one of the modified therapeutic cells or methods described above, the therapeutic cell is an immune cell. In some embodiments, the immune cell is selected from the group consisting of cytotoxic T cell, helper T cell, NK cell, NK-T cell, αβ T cell, γδ T cell, tumor-infiltrating T cell, dendritic cell (DC)-activated T cell, and peripheral blood mononuclear cell (PBMC). In some embodiments, the therapeutic cell is a T cell. In some embodiments, the therapeutic cell is a γδ T cell. In some embodiments, the therapeutic cell is a γ9δ2 T cell. In some embodiments, the therapeutic cell is a δ1 T cell. In some embodiments, the therapeutic cell is a δ3 T cell. In some embodiments, the therapeutic cell is a stem cell. In some embodiments, the therapeutic cell is an embryonic stem cell (ESC). In some embodiments, the therapeutic cell is a hematopoietic stem cell (HSC).

In some embodiments according to any one of the modified therapeutic cells or methods described above, the therapeutic cell further comprises a second heterologous nucleic acid sequence encoding an engineered receptor. In some embodiments, the engineered receptor is selected from the group consisting of a chimeric antigen receptor (CAR), a recombinant T cell receptor (TCR), a T-cell antigen coupler (TAC) receptor and a TCR fusion protein (TFP). In some embodiments, the therapeutic cell is a CAR-T cell. In some embodiments, the engineered receptor targets a tumor antigen selected from the group consisting of CD19, BCMA, NY-ESO-1, VEGFR2, MAGE-A3, CD20, CD22, CD33, CD38, CEA, EGFR, GD2, HER2, IGF1R, mesothelin, PSMA, ROR1, WT1 and combinations thereof. In some embodiments, the engineered receptor is an anti-BCMA CAR. In some embodiments, the engineered receptor is an anti-CD19 CAR. In some embodiments, the therapeutic cell does not have genetic modification other than the first and second heterologous nucleic acid sequences. In some embodiments, the first heterologous nucleic acid sequence and the second heterologous nucleic acid sequence are operably linked to the same promoter or different promoters. In some embodiments, the first heterologous nucleic acid sequence and the second heterologous nucleic acid sequence are present in a vector (e.g., viral vector such as lentiviral vector). In some embodiments, the first heterologous nucleic acid sequence is fused to the second heterologous nucleic acid sequence via a third nucleic acid sequence encoding a self-cleavable linker. In some embodiments, the modified therapeutic cell comprises a nucleic acid sequence encoding an amino acid sequence selected from the group consisting of SEQ ID NOs: 12-27.

In some embodiments according to any one of the modified therapeutic cells or methods described above, the ICL is expressed at an increased level compared to an unmodified therapeutic cell.

One aspect of the present application provides a pharmaceutical composition comprising the modified therapeutic cell according to any one of the modified therapeutic cells described above.

One aspect of the present application provides a method of treating a disease or condition in an individual in need thereof, comprising administering to the individual an effective amount of the pharmaceutical composition according to afy one of the pharmaceutical compositions described above.

In some embodiments according to any one of the methods described above, the modified therapeutic cell is allogeneic. In some embodiments, the individual is human.

In some embodiments according to any one of the methods described above, one or more human leukocyte antigen (HLA) alleles of the individual have mismatching allotypes compared to those of the modified therapeutic cell. In some embodiments, one or more (e.g., 8 out of 8, 7 out of 8, or 6 out of 8) alleles of HLA-A, HLA-B, HLA-C and HLA-DRB1 have mismatching allotypes compared to those of the modified therapeutic cell. In some embodiments, all tested HLA alleles of the individual have matching allotypes compared to those of the modified therapeutic cell.

In some embodiments according to any one of the methods described above, the method reduces undesired immune response against the therapeutic cell in the individual compared to a method using a therapeutic cell that does not comprise the first heterologous nucleic acid sequence encoding the ICL. In some embodiments, the undesired immune response comprises Host-versus-Graft (HvG) response.

In some embodiments according to any one of the methods described above, the method induces immune tolerance towards the therapeutic cell in the individual compared to a method using a therapeutic cell that does not comprise the first heterologous nucleic acid sequence encoding the ICL.

In some embodiments according to any one of the methods described above, the disease or condition is a cancer, an infectious disease, or an autoimmune disease.

One aspect of the present application provides a kit comprising the modified therapeutic cell according to any one of the modified therapeutic cells described above and instructions for treating a disease or condition in an individual in need thereof. In some embodiments, the modified therapeutic cell is allogeneic. In some embodiments, the modified therapeutic cell is an allogeneic CAR-T cell. In some embodiments, the modified therapeutic cell is an allogeneic CAR-NK cell. In some embodiments, the modified therapeutic cell is an allogeneic CAR-NKT cell. In some embodiments, the modified therapeutic cell is an allogeneic CAR-γδ T cell.

Compositions, uses, kits and articles of manufacture comprising any one of the modified therapeutic cells described herein are also provided.

### BRIEF DESCRIPTION OF THE DRAWINGS

FIGS. 1A-1B show schematics of constructs for therapeutic immune cells (FIG. 1A) and CAR-modified immune cells (FIG. 1B), which overexpress an immune checkpoint protein (ICP) ligand to reduce graft rejection. The ICP ligands can be PD-L1, CD155, CD112, FGL1, galectin-9, CD47, B7H3 or B7H4. The transgene encoding the immune checkpoint protein ligand can be placed either upstream or downstream of the transgene encoding the CAR. The construct in FIG. 1B is a contiguous piece of nucleic acid encoding both CAR and ICP.

FIGS. 2A-2D show expression level of anti-BCMA CAR, BSF17, or anti-CD19 CAR, CTL-019, alongside ICL ligand, PD-L1, on αβ or γδ T cells.

FIGS. 3A-3F show short-term in vitro killing level and long-term persistence of anti-BCMA CAR, BSF17, or anti-CD19 CAR, CTL-019, alongside ICL ligand, PD-L1, on αβ or γδ T cells.

FIGS. 4A-4C show the level of IFN-γ and TNF-α production anti-BCMA CAR, BSF17, or anti-CD19 CAR, CTL-019, alongside ICL ligand, PD-L1, on αβ or γδ T cells.

FIGS. 5A-5J show one-way MLR results displayed by the level of allogeneic αβ T or NK cells proliferation, with control groups, stimulated by anti-BCMA CAR, BSF17, or anti-CD19 CAR, CTL-019, alongside ICL ligand, PD-L1, on αβ or γδ T cells.

FIGS. 6A-6C show in vivo anti-tumor efficacy and proliferation of anti-BCMA CAR, BSF17, alongside ICL ligand, PD-L1, on γδ T cells in an RPMI-8226 (myeloma) xenograft model.

### DETAILED DESCRIPTION OF THE INVENTION

The present application provides therapeutic cells and methods of treatment associated with reduced undesired immune response by overexpressing an immune checkpoint ligand (ICL) in the therapeutic cells without suppressing the entire host immune system or eliciting innate immune response against the therapeutic cells. In some embodiments, the therapeutic cells are allogeneic immune cells or stem cells. The compositions and methods described herein are applicable to a wide range of adoptive cell therapy, including therapy using adaptive immunity cells such as αβ T cells, γδ T cells, and Natural Killer T (NKT) cells, innate immunity cells such as NK cells, stem cells such as hematopoietic stem cells (HSC), induced pluripotent stem cells (iPSC) and embryonic stem cells (ESC), as well as products modified with engineered receptors such as Chimeric Antigen Receptor (CAR) modified.

The present application is based at least in part on the inventor's observation that immune cells overexpressing an ICL can suppress T cell proliferation in MHC-mismatched PBMCs, but immune cells co-cultured with MHC-mismatched PBMCs in the presence of soluble ICL do not show similar immune-suppressive effects. Without being bound by any theory or hypothesis, when CAR-T cells do not express MHC complexes, they are not be recognized by host T cells, but such CAR-T cells can be recognized and cleared by NK cells. The compositions and methods described herein can reduce graft rejection by both T cells and NK cells in the patient, induce peripheral immune tolerance in the patient, and increase efficacy of the cell therapy.

Accordingly, one aspect of the present application provides a modified therapeutic cell comprising a first heterologous nucleic acid sequence encoding an immune checkpoint ligand (ICL), wherein the therapeutic cell expresses a Major Histocompatibility Complex (MHC) molecule. In some embodiments, the ICL is PD-L1, CD155, CD112, FGL1, galectin-9, CD47, B7H3 or B71-14.

One aspect of the present application provides a method of reducing graft rejection of allogeneic therapeutic cells in an individual in need thereof, comprising administering to the individual an effective amount of the allogeneic therapeutic cells, wherein the allogeneic therapeutic cells comprise a first heterologous nucleic acid sequence encoding an immune checkpoint ligand (ICL), and wherein the therapeutic cells express an MHC molecule.

### I. Definitions

As used herein, “immune checkpoint,” “immune checkpoint protein,” or “ICP” are used herein interchangeably to refer to a molecule in the immune system that either turns up (stimulatory molecules) or turns down a signal (inhibitory molecules). Immune checkpoint proteins regulate and maintain self-tolerance and the duration and amplitude of physiological immune responses. Stimulatory checkpoint molecules include, but are not limited to, CD27, CD40, OX40, GITR and CD137, which belong to tumor necrosis factor (TNF) receptor superfamily, as well as CD28 and ICOS, which belong to the B7-CD28 superfamily. Inhibitory checkpoint molecules include, but are not limited to, program death 1 (PD-1), Cytotoxic T-Lymphocyte-Associated protein 4 (CTLA-4), Lymphocyte Activation Gene-3 (LAG-3), T-cell Immunoglobulin domain and Mucin domain 3 (TIM-3), V-domain Ig suppressor of T cell activation (VISTA), B7-H3, B7-H4, B and T Lymphocyte Attenuator (BTLA), Indoleamine 2,3-dioxygenase (IDO), Killer-cell Immunoglobulin-like Receptor (KIR), adenosine A2A receptor, and ligands thereof. Numerous checkpoint proteins have been studied extensively, such as CTLA-4 and its ligands CD80 and CD86, and PD-1 with its ligands PD-L1 and PD-L2 (See, for example, Pardoll, Nature Reviews Cancer 12: 252-264 (2012)).

The term “immune checkpoint ligand,” “immune checkpoint protein ligand” or “ICL” refers to a natural or engineered ligand that specifically binds to an immune checkpoint molecule. In some embodiments, the ICL is membrane bound. In some embodiments, the ICL is a soluble protein.

As used herein, “treatment” or “treating” is an approach for obtaining beneficial or desired results including clinical results. For purposes of this invention, beneficial or desired clinical results include, but are not limited to, one or more of the following: alleviating one or more symptoms resulting from the disease, diminishing the extent of the disease, stabilizing the disease (e.g., preventing or delaying the worsening of the disease), preventing or delaying the spread (e.g., metastasis) of the disease, preventing or delaying the recurrence of the disease, delay or slowing the progression of the disease, ameliorating the disease state, providing a remission (partial or total) of the disease, decreasing the dose of one or more other medications required to treat the disease, delaying the progression of the disease, increasing the quality of life, and/or prolonging survival. Also encompassed by “treatment” is a reduction of pathological consequence of the disease. The methods of the present application contemplate any one or more of these aspects of treatment.

The term “prevent,” and similar words such as “prevented,” “preventing” etc., indicate an approach for preventing, inhibiting, or reducing the likelihood of the recurrence of, a disease or condition, e.g., cancer. It also refers to delaying the recurrence of a disease or condition or delaying the recurrence of the symptoms of a disease or condition. As used herein, “prevention” and similar words also includes reducing the intensity, effect, symptoms and/or burden of a disease or condition prior to recurrence of the disease or condition.

As used herein, “delaying” the development of a disease means to defer, hinder, slow, retard, stabilize, and/or postpone development of the disease. This delay can be of varying lengths of time, depending on the history of the disease and/or individual being treated. A method that “delays” development of a disease is a method that reduces probability of disease development in a given time frame and/or reduces the extent of the disease in a given time frame, when compared to not using the method. Such comparisons are typically based on clinical studies, using a statistically significant number of individuals. Disease development can be detectable using standard methods, including, but not limited to, computerized axial tomography (CAT Scan), Magnetic Resonance Imaging (MRI), abdominal ultrasound, clotting tests, arteriography, or biopsy. Development may also refer to disease progression that may be initially undetectable and includes occurrence, recurrence, and onset.

The term “effective amount” used herein refers to an amount of an agent sufficient to treat a specified disorder, condition or disease such as ameliorate, palliate, lessen, and/or delay one or more of its symptoms. In reference to cancer, an effective amount comprises an amount sufficient to cause a tumor to shrink and/or to decrease the growth rate of the tumor (such as to suppress tumor growth) or to prevent or delay other unwanted cell proliferation. In some embodiments, an effective amount is an amount sufficient to delay development. In some embodiments, an effective amount is an amount sufficient to prevent or delay recurrence. An effective amount can be administered in one or more administrations. The effective amount of the drug or composition may: (i) reduce the number of cancer cells; (ii) reduce tumor size; (iii) inhibit, retard, slow to some extent and preferably stop cancer cell infiltration into peripheral organs; (iv) inhibit (i.e., slow to some extent and preferably stop) tumor metastasis, (v) inhibit tumor growth; (vi) prevent or delay occurrence and/or recurrence of tumor; and/or (vii) relieve to some extent one or more of the symptoms associated with the cancer.

As used herein, an “individual” or a “subject” refers to a mammal, including, but not limited to, human, bovine, horse, feline, canine, rodent, or primate. In some embodiments, the individual is a human.

As use herein, the term “binds”, “specifically binds to” or is “specific for” refers to measurable and reproducible interactions such as binding between a target and a ligand, which is determinative of the presence of the target in the presence of a heterogeneous population of molecules including biological molecules. For example, a ligand that binds to or specifically binds to a target is a ligand that binds this target with greater affinity, avidity, more readily, and/or with greater duration than it binds to other targets. In one embodiment, the extent of binding of a ligand to an unrelated target is less than about 10% of the binding of the ligand to the target as measured, e.g., by a radioimmunoassay (RIA). In certain embodiments, a ligand that specifically binds to a target has a dissociation constant (Kd) of ≤1 μM, ≤100 nM, ≤10 nM, ≤1 nM, or ≤0.1 nM. In certain embodiments, a ligand specifically binds to a region on a protein that is conserved among the proteins from different species. In another embodiment, specific binding can include, but does not require exclusive binding.

The term “antibody” includes monoclonal antibodies, antibody compositions with polyepitopic specificity, multispecific antibodies (e.g., bispecific antibodies, diabodies, and single-chain molecules), as well as antibody fragments (e.g., Fab, F(ab′)₂, and Fv). The term “immunoglobulin” (Ig) is used interchangeably with “antibody” herein. The term antibody includes conventional four-chain antibodies, and single-domain antibodies, such as heavy-chain only antibodies or fragments thereof, e.g., V_{H}H.

“Chimeric antigen receptor” or “CAR” as used herein refers to engineered receptors, which can be used to graft one or more antigen specificity onto immune effector cells, such as T cells. Some CARs are also known as “artificial T-cell receptors,” “chimeric T cell receptors,” or “chimeric immune receptors.” In some embodiments, the CAR comprises an extracellular antigen binding domain specific for one or more antigens (such as tumor antigens), a transmembrane domain, and an intracellular signaling domain of a T cell and/or other receptors. “CAR-T” refers to a T cell that expresses a CAR.

“UCAR-T” or “universal CAR-T cells” refers to off-the-shelf CAR-modified T cells that can be used to treat an allogeneic patient in need thereof. UCAR-Ts include those that contain genetic modification in addition to the CAR construct, and those that do not contain genetic modification other than the CAR construct.

“T cell receptor” or “TCR” as used herein refers to endogenous or recombinant T cell receptor comprising an extracellular antigen-binding domain that binds to a specific antigenic peptide bound in an MHC molecule. In some embodiments, the TCR comprises a TCRα polypeptide chain and a TCR β polypeptide chain. In some embodiments, the TCR specifically binds a tumor antigen. “TCR-T” refers to a T cell that expresses a recombinant TCR.

“T-cell antigen coupler receptor” or “TAC receptor” as used herein refers to an engineered receptor comprising an extracellular antigen-binding domain that binds to a specific antigen and a T-cell receptor (TCR) binding domain, a transmembrane domain, and an intracellular domain of a co-receptor molecule. The TAC receptor co-opts the endogenous TCR of a T cell that expressed the TAC receptor to elicit antigen-specific T-cell response against a target cell.

“TCR fusion protein” or “TFP” as used herein refers to an engineered receptor comprising an extracellular antigen-binding domain that binds to a specific antigen fused to a subunit of the TCR complex or a portion thereof, including TCRα chain, TCRβ chain, TCRγ chain, TCRδ chain, CD3ε, CD3δ, or CD3γ. The subunit of the TCR complex or portion thereof comprise a transmembrane domain and at least a portion of the intracellular domain of the naturally occurring TCR subunit. In some embodiments, the TFP comprises the extracellular domain of the TCR subunit or a portion thereof. In some embodiments, the TFP does not comprise the extracellular domain of the TCR subunit.

“Percent (%) amino acid sequence identity” and “homology” with respect to a polypeptide sequence are defined as the percentage of amino acid residues in a candidate sequence that are identical with the amino acid residues in the specific polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms needed to achieve maximal alignment over the full length of the sequences being compared. For example, polypeptides having at least 70%, 85%, 90%, 95%, 98% or 99% identity to specific polypeptides described herein and preferably exhibiting substantially the same functions, as well as polynucleotide encoding such polypeptides, are contemplated.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

The term “transfected” or “transformed” or “transduced” as used herein refers to a process by which exogenous nucleic acid is transferred or introduced into the host cell. A “transfected” or “transformed” or “transduced” cell is a cell, which has been transfected, transformed or transduced with exogenous nucleic acid.

As used herein, the expressions “cell”, “cell line”, and “cell culture” are used interchangeably and all such designations include progeny. Thus, the words “transfectants” and “transfected cells” include the primary subject cell and cultures derived there from without regard for the number of transfers. It is also understood that not all progeny may be precisely identical in DNA content, due to deliberate or inadvertent mutations. Variant progeny that have the same function or biological activity as screened for in the originally transformed cell are included.

“Primary cells” refer to cells taken directly from living tissue (i.e. biopsy material) and established for growth in vitro, which have undergone very few population doublings and are therefore more representative of the main functional components and characteristics of tissues from which they are derived from, in comparison to continuous tumorigenic or artificially immortalized cell lines.

The term “in vivo” refers to inside the body of the organism from which the cell is obtained. “Ex vivo” or “in vitro” means outside the body of the organism from which the cell is obtained.

As used herein, the term “autologous” refers to any material derived from the same individual to whom it is later to be re-introduced into the individual.

“Allogeneic” refers to a graft derived from a different individual of the same species.

The term “recombinant” refers to a biomolecule, e.g., a gene or protein, that (1) has been removed from its naturally occurring environment, (2) is not associated with all or a portion of a polynucleotide in which the gene is found in nature, (3) is operatively linked to a polynucleotide which it is not linked to in nature, or (4) does not occur in nature. The term “recombinant” can be used in reference to cloned DNA isolates, chemically synthesized polynucleotide analogs, or polynucleotide analogs that are biologically synthesized by heterologous systems, as well as proteins and/or mRNAs encoded by such nucleic acids.

The term “express” refers to transcription of a DNA to an RNA (e.g., mRNA), or translation of a nucleic acid into a protein. Proteins may be expressed and remain intracellular, become a component of the cell surface membrane, or be secreted into extracellular matrix or medium.

It is understood that embodiments of the present application described herein include “consisting” and/or “consisting essentially of” embodiments.

Reference to “about” a value or parameter herein includes (and describes) variations that are directed to that value or parameter per se. For example, description referring to “about X” includes description of “X”.

As used herein, reference to “not” a value or parameter generally means and describes “other than” a value or parameter. For example, the method is not used to treat cancer of type X means the method is used to treat cancer of types other than X.

The term “about X-Y” used herein has the same meaning as “about X to about Y.”

As used herein and in the appended claims, the singular forms “a”, “an”, and “the” include plural referents unless the context clearly dictates otherwise.

### II. Modified Therapeutic Cells

The present application provides a modified therapeutic cell (e.g., allogeneic immune cell or stem cell) associated with reduced undesired immune response, such as reduced graft rejection or Host-versus-Graft Disease (HvGD), increased immune tolerance, and/or increased therapeutic efficacy. The modified therapeutic cells described herein comprise heterologous nucleic acid sequence(s) encoding one or more immune checkpoint ligands, and express endogenous Major Histocompatibility (MHC) molecule(s).

In some embodiments, there is provided a modified therapeutic cell comprising a first heterologous nucleic acid sequence encoding an ICL, wherein the therapeutic cell expresses an MHC molecule. In some embodiments, the therapeutic cell expresses an MHC class I molecule. In some embodiments, the therapeutic cell expresses an MHC class II molecule. In some embodiments, the therapeutic cell expresses both an MHC class I molecule and an MHC class II molecule. In some embodiments, the β2-microglobulin (B2M) gene of the therapeutic cell is not genetically modified. In some embodiments, the MHC genes of the therapeutic cell are not genetically modified.

Suitable ICL molecules include, but are not limited to, PD-L1, CD155, CD112, FGL1, galectin-9, CD47, B7H3 and B7114. In some embodiments, the therapeutic cell comprises a first heterologous nucleic acid sequence encoding an ICL selected from the group consisting of PD-L1, CD155, CD112, FGL1, galectin-9, CD47, B7H3 and B7H4. In some embodiments, the therapeutic cell comprises a first heterologous nucleic acid sequence encoding a single ICL. In some embodiments, the therapeutic cell comprises a first heterologous nucleic acid sequence encoding two or more ICLs, such as any one of 2, 3, 4, 5, 6, or more ICLs. Exemplary ICLs are described in the subsection “A. Immune checkpoint ligands” below.

In some embodiments, the ICL is expressed at an increased level compared to an unmodified therapeutic cell. For example, the ICL is expressed in the modified therapeutic cell at a level that is at least about any one of 10%, 20%, 30%, 40%, 50%, 60%, 70%, 80%, 90%, 1 fold, 1.5 fold, 2 fold, 2.5 fold, 3 fold, 4 fold, 5 fold, 10 fold, 20 fold, 50 fold, 100 fold or more, including any levels and ranges in between these levels, higher than the level of the ICL in an unmodified therapeutic cell. In some embodiments, the ICL is expressed in the modified therapeutic cell at a level that is about any one of 10%-50%, 50%-100%, 100-100%, 1 fold to 2 fold, 2 fold to 5 fold, 5 fold to 10 fold, 1 fold to 10 fold, 10 fold to 50 fold, or 1 fold to 100 fold, higher than the level of the ICL in an unmodified therapeutic cell. In some embodiments, the ICL expression level is mRNA level. In some embodiments, the ICL expression level is protein level. The ICL expression level can be measured using any known methods in the art. For example, mRNA expression level can be determined using quantitative polymerase chain reaction (qPCR), or fluorescence in situ hybridization (FISH) assays. Protein expression levels can be determined using Western blots, enzyme-linked immunosorbent assays (ELISA), or reporter assays.

In some embodiments, there is provided a modified therapeutic cell comprising a first heterologous nucleic acid sequence encoding PD-L1, wherein the therapeutic cell expresses an MHC molecule. In some embodiments, the therapeutic cell expresses an MHC class I molecule. In some embodiments, the therapeutic cell expresses an MHC class II molecule. In some embodiments, the therapeutic cell expresses both an MHC class I molecule and an MHC class II molecule. In some embodiments, the β2-microglobulin (B2M) gene of the therapeutic cell is not genetically modified. In some embodiments, the MHC genes of the therapeutic cell are not genetically modified. In some embodiments, the ICL comprises an amino acid sequence having at least about 85% (e.g., about 90%, 95%, 98%, or more) sequence identity to the amino acid sequence of SEQ ID NO: 1. In some embodiments, the ICL comprises the amino acid sequence of SEQ ID NO: 1.

In some embodiments, there is provided a modified γδ T cell comprising a first heterologous nucleic acid sequence encoding PD-L1, wherein the γδ T cell expresses an MHC molecule. In some embodiments, the γδ T cell expresses an MHC class I molecule. In some embodiments, the γδ T cell expresses an MHC class II molecule. In some embodiments, the γδ T cell expresses both an MHC class I molecule and an MHC class II molecule. In some embodiments, the β2-microglobulin (B2M) gene of the γδ T cell is not genetically modified. In some embodiments, the MHC genes of the γδ T cell are not genetically modified. In some embodiments, the ICL comprises an amino acid sequence having at least about 85% (e.g., about 90%, 95%, 98%, or more) sequence identity to the amino acid sequence of SEQ ID NO: 1. In some embodiments, the ICL comprises the amino acid sequence of SEQ ID NO: 1. In some embodiments, the γδ T cell is a γ9δ2 T cell. In some embodiments, the γδ T cell is a δ1 T cell. In some embodiments, the γδ T cell is a δ3 T cell.

In some embodiments, there is provided a modified γδ T cell comprising a first heterologous nucleic acid sequence encoding PD-L1, and a second heterologous nucleic acid sequence encoding an engineered receptor (e.g., a CAR), wherein the γδ T cell expresses an MHC molecule. In some embodiments, the γδ T cell expresses an MHC class I molecule. In some embodiments, the γδ T cell expresses an MHC class II molecule. In some embodiments, the γδ T cell expresses both an MHC class I molecule and an MHC class II molecule. In some embodiments, the δ2-microglobulin (B2M) gene of the γδ T cell is not genetically modified. In some embodiments, the MHC genes of the γδ T cell are not genetically modified. In some embodiments, the ICL comprises an amino acid sequence having at least about 85% (e.g., about 90%, 95%, 98%, or more) sequence identity to the amino acid sequence of SEQ ID NO: 1. In some embodiments, the ICL comprises the amino acid-sequence of SEQ ID NO: 1. In some embodiments, the engineered receptor targets a tumor antigen selected from the group consisting of CD19, BCMA, NY-ESO-1, VEGFR2, MAGE-A3, CD20, CD22, CD33, CD38, CEA, EGFR, GD2, HER2, IGF1R, mesothelin, PSMA, ROR1, WT1 and combinations thereof. In some embodiments, the engineered receptor is selected from the group consisting of a CAR, a TCR, a TAC receptor and a TFP. In some embodiments, the engineered receptor is a CAR. In some embodiments, the CAR is an anti-BCMA CAR. In some embodiments, the CAR comprises from the N-terminus to the C-terminus, an anti-BCMA antigen-binding moiety (e.g., a VHH), a hinge region, a transmembrane domain, a co-stimulatory domain (e.g., CD28 or 4-1BB) and a CD3ζ intracellular signaling domain. In some embodiment, the anti-BCMA CAR is BSF17 CAR. In some embodiments, the CAR is an anti-CD19 CAR. In some embodiments, the CAR comprises from the N-terminus to the C-terminus, an anti-CD19 antigen-binding moiety (e.g., a scFv), a hinge region, a transmembrane domain, a co-stimulatory domain (e.g., CD28 or 4-1BB) and a CD3ζ intracellular signaling domain. In some embodiments, the anti-CD19 CAR is CTL-019 CAR. In some embodiments, the γδ T is a γ9δ2 T cell. In some embodiments, the γδ T cell is a δ1 T cell. In some embodiments, the γδ T cell is a δ3 T cell.

In some embodiments, there is provided a modified therapeutic cell comprising a first heterologous nucleic acid sequence encoding CD155, wherein the therapeutic cell expresses an MHC molecule. In some embodiments, the therapeutic cell expresses an MHC class I molecule. In some embodiments, the therapeutic cell expresses an MHC class II molecule. In some embodiments, the therapeutic cell expresses both an MHC class I molecule and an MHC class II molecule. In some embodiments, the β2-microglobulin (B2M) gene of the therapeutic cell is not genetically modified. In some embodiments, the MHC genes of the therapeutic cell are not genetically modified. In some embodiments, the ICL comprises an amino acid sequence having at least about 85% (e.g., about 90%, 95%, 98%, or more) sequence identity to the amino acid sequence of SEQ ID NO: 2. In some embodiments, the ICL comprises the amino acid sequence of SEQ ID NO: 2.

In some embodiments, there is provided a modified γδ T cell comprising a first heterologous nucleic acid sequence encoding CD155, wherein the γδ T cell expresses an MHC molecule. In some embodiments, the γδ T cell expresses an MHC class I molecule. In some embodiments, the γδ T cell expresses an MHC class II molecule. In some embodiments, the 16 T cell expresses both an MHC class I molecule and an MHC class II molecule. In some embodiments, the β2-microglobulin (B2M) gene of the γδ T cell is not genetically modified. In some embodiments, the MHC genes of the γδ T cell are not genetically modified. In some embodiments, the ICL comprises an amino acid sequence having at least about 85% (e.g., about 90%, 95%, 98%, or more) sequence identity to the amino acid sequence of SEQ ID NO: 2. In some embodiments, the ICL comprises the amino acid sequence of SEQ ID NO: 2. In some embodiments, the γδ T is a γ9δ2 T cell. In some embodiments, the γδ T cell is a δ1 T cell. In some embodiments, the γδ T cell is a δ3 T cell.

In some embodiments, there is provided a modified γδ T cell comprising a first heterologous nucleic acid sequence encoding CD155, and a second heterologous nucleic acid sequence encoding an engineered receptor (e.g., a CAR), wherein the γδ T cell expresses an MHC molecule. In some embodiments, the γδ T cell expresses an MHC class I molecule. In some embodiments, the γδ T cell expresses an MHC class II molecule. In some embodiments, the γδ T cell expresses both an MHC class I molecule and an MHC class II molecule. In some embodiments, the β2-microglobulin (B2M) gene of the γδ T cell is not genetically modified. In some embodiments, the MHC genes of the γδ T cell are not genetically modified. In some embodiments, the ICL comprises an amino acid sequence having at least about 85% (e.g., about 90%, 95%, 98%, or more) sequence identity to the amino acid sequence of SEQ ID NO: 2. In some embodiments, the ICL comprises the amino acid sequence of SEQ ID NO: 2. In some embodiments, the engineered receptor targets a tumor antigen selected from the group consisting of CD19, BCMA, NY-ESO-1, VEGFR2, MAGE-A3, CD20, CD22, CD33, CD38, CEA, EGFR, GD2, HER2, IGF1R, mesothelin, PSMA, ROR1, WT1 and combinations thereof. In some embodiments, the engineered receptor is selected from the group consisting of a CAR, a TCR, a TAC receptor and a TFP. In some embodiments, the engineered receptor is a CAR. In some embodiments, the CAR is an anti-BCMA CAR. In some embodiments, the CAR is an anti-CD19 CAR. In some embodiments, the γδ T is a γ9δ2 T cell. In some embodiments, the γδ T cell is a δ1 T cell. In some embodiments, the γδ T cell is a δ3 T cell.

In some embodiments, there is provided a modified therapeutic cell comprising a first heterologous nucleic acid sequence encoding CD112, wherein the therapeutic cell expresses an NHC molecule. In some embodiments, the therapeutic cell expresses an MHC class I molecule. In some embodiments, the therapeutic cell expresses an MHC class II molecule. In some embodiments, the therapeutic cell expresses both an MHC class I molecule and an MHC class II molecule. In some embodiments, the β2-microglobulin (B2M) gene of the therapeutic cell is not genetically modified. In some embodiments, the MHC genes of the therapeutic cell are not genetically modified. In some embodiments, the ICL comprises an amino acid sequence having at least about 85% (e.g., about 90%, 95%, 98%, or more) sequence identity to the amino acid sequence of SEQ ID NO: 3. In some embodiments, the ICL comprises the amino acid sequence of SEQ ID NO: 3.

In some embodiments, there is provided a modified γδ T cell comprising a first heterologous nucleic acid sequence encoding CD112, wherein the γδ T cell expresses an MHC molecule. In some embodiments, the γδ T cell expresses an MHC class I molecule. In some embodiments, the γδ T cell expresses an MHC class II molecule. In some embodiments, the γδ T cell expresses both an MHC class I molecule and an MHC class II molecule. In some embodiments, the β2-microglobulin (B2M) gene of the γδ T cell is not genetically modified. In some embodiments, the MHC genes of the γδ T cell are not genetically modified. In some embodiments, the ICL comprises an amino acid sequence having at least about 85% (e.g., about 90%, 95%, 98%, or more) sequence identity to the amino acid sequence of SEQ ID NO: 3. In some embodiments, the ICL comprises the amino acid sequence of SEQ ID NO: 3. In some embodiments, the γδ T is a γ9δ2 T cell. In some embodiments, the γδ T cell is a δ1 T cell. In some embodiments, the γδ T cell is a δ3 T cell.

In some embodiments, there is provided a modified γδ T cell comprising a first heterologous nucleic acid sequence encoding CD112, and a second heterologous nucleic acid sequence encoding an engineered receptor (e.g., a CAR), wherein the γδ T cell expresses an MHC molecule. In some embodiments, the γδ T cell expresses an MHC class I molecule. In some embodiments, the γδ T cell expresses an MHC class II molecule. In some embodiments, the γδ T cell expresses both an MHC class I molecule and an MHC class II molecule. In some embodiments, the β2-microglobulin (B2M) gene of the γδ T cell is not genetically modified. In some embodiments, the MHC genes of the γδ T cell are not genetically modified. In some embodiments, the ICL comprises an amino acid sequence having at least about 85% (e.g., about 90%, 95%, 98%, or more) sequence identity to the amino acid sequence of SEQ ID NO: 3. In some embodiments, the ICL comprises the amino acid sequence of SEQ ID NO: 3. In some embodiments, the engineered receptor targets a tumor antigen selected from the group consisting of CD19, BCMA, NY-ESO-1, VEGFR2, MAGE-A3, CD20, CD22, CD33, CD38, CEA, EGFR, GD2, HER2, IGF1R, mesothelin, PSMA, ROR1, WT1 and combinations thereof. In some embodiments, the engineered receptor is selected from the group consisting of a CAR, a TCR, a TAC receptor and a TFP. In some embodiments, the engineered receptor is a CAR. In some embodiments, the CAR is an anti-BCMA CAR. In some embodiments, the CAR is an anti-CD19 CAR. In some embodiments, the γδ T is a γ9δ2 T cell. In some embodiments, the γδ T cell is a δ1 T cell. In some embodiments, the γδ T cell is a δ3 T cell.

In some embodiments, there is provided a modified therapeutic cell comprising a first heterologous nucleic acid sequence encoding FGL1, wherein the therapeutic cell expresses an MHC molecule. In some embodiments, the therapeutic cell expresses an MHC class I molecule. In some embodiments, the therapeutic cell expresses an MHC class II molecule. In some embodiments, the therapeutic cell expresses both an MHC class I molecule and an MHC class II molecule. In some embodiments, the β2-microglobulin (B2M) gene of the therapeutic cell is not genetically modified. In some embodiments, the MHC genes of the therapeutic cell are not genetically modified. In some embodiments, the ICL comprises an amino acid sequence having at least about 85% (e.g., about 90%, 95%, 98%, or more) sequence identity to the amino acid sequence of SEQ ID NO: 4. In some embodiments, the ICL comprises the amino acid sequence of SEQ ID NO: 4.

In some embodiments, there is provided a modified γδ T cell comprising a first heterologous nucleic acid sequence encoding FGL1, wherein the γδ T cell expresses an MHC molecule. In some embodiments, the γδ T cell expresses an MHC class I molecule. In some embodiments, the γδ T cell expresses an MHC class II molecule. In some embodiments, the γδ T cell expresses both an MHC class I molecule and an MHC class II molecule. In some embodiments, the β2-microglobulin (B2M) gene of the γδ T cell is not genetically modified. In some embodiments, the MHC genes of the γδ T cell are not genetically modified. In some embodiments, the ICL comprises an amino acid sequence having at least about 85% (e.g., about 90%, 95%, 98%, or more) sequence identity to the amino acid sequence of SEQ ID NO: 4. In some embodiments, the ICL comprises the amino acid sequence of SEQ ID NO: 4. In some embodiments, the γδ T is a γ9 δ2 T cell. In some embodiments, the γδ cell is a 61 T cell. In some embodiments, the γδ T cell is a δ3 T cell.

In some embodiments, there is provided a modified γδ T cell comprising a first heterologous nucleic acid sequence encoding FGL1, and a second heterologous nucleic acid sequence encoding an engineered receptor (e.g., a CAR), wherein the γδ T cell expresses an MHC molecule. In some embodiments, the γδ T cell expresses an MHC class I molecule. In some embodiments, the γδ T cell expresses an MHC class II molecule. In some embodiments, the γδ T cell expresses both an MHC class I molecule and an MHC class II molecule. In some embodiments, the β2-microglobulin (B2M) gene of the γδ T cell is not genetically modified. In some embodiments, the MHC genes of the γδ T cell are not genetically modified. In some embodiments, the ICL comprises an amino acid sequence having at least about 85% (e.g., about 90%, 95%, 98%, or more) sequence identity to the amino acid sequence of SEQ ID NO: 4. In some embodiments, the ICL comprises the amino acid sequence of SEQ ID NO: 4. In some embodiments, the engineered receptor targets a tumor antigen selected from the group consisting of CD19, BCMA, NY-ESO-1, VEGFR2, MAGE-A3, CD20, CD22, CD33, CD38, CEA, EGFR, GD2, HER2, IGF1R, mesothelin, PSMA, ROR1, WT1 and combinations thereof. In some embodiments, the engineered receptor is selected from the group consisting of a CAR, a TCR, a TAC receptor and a TFP. In some embodiments, the engineered receptor is a CAR. In some embodiments, the CAR is an anti-BCMA CAR. In some embodiments, the CAR is an anti-CD19 CAR. In some embodiments, the γδ T is a γ9δ2 T cell. In some embodiments, the γδ T cell is a δ1 T cell. In some embodiments, the γδ T cell is a δ3 T cell.

In some embodiments, there is provided a modified therapeutic cell comprising a first heterologous nucleic acid sequence encoding galectin-9, wherein the therapeutic cell expresses an MHC molecule. In some embodiments, the therapeutic cell expresses an MHC class I molecule. In some embodiments, the therapeutic cell expresses an MHC class II molecule. In some embodiments, the therapeutic cell expresses both an MHC class I molecule and an MHC class II molecule. In some embodiments, the β2-microglobulin (B2M) gene of the therapeutic cell is not genetically modified. In some embodiments, the MHC genes of the therapeutic cell are not genetically modified. In some embodiments, the ICL comprises an amino acid sequence having at least about 85% (e.g., about 90%6, 95%, 98%, or more) sequence identity to the amino acid sequence of SEQ ID NO: 5. In some embodiments, the ICL comprises the amino acid sequence of SEQ ID NO: 5.

In some embodiments, there is provided a modified γδ T cell comprising a first heterologous nucleic acid sequence encoding galectin-9, wherein the γδ T cell expresses an MHC molecule. In some embodiments, the γδ T cell expresses an MHC class I molecule. In some embodiments, the γδ T cell expresses an MHC class II molecule. In some embodiments, the γδ T cell expresses both an MHC class I molecule and an MHC class II molecule. In some embodiments, the β2-microglobulin (B2M) gene of the γδ T cell is not genetically modified. In some embodiments, the MHC genes of the γδ T cell are not genetically modified. In some embodiments, the ICL comprises an amino acid sequence having at least about 85% (e.g., about 90%, 95%, 98%, or more) sequence identity to the amino acid sequence of SEQ ID NO: 5. In some embodiments, the ICL comprises the amino acid sequence of SEQ ID NO: 5. In some embodiments, the γδ T is a γ9δ2 T cell. In some embodiments, the γδ T cell is a δ1 T cell. In some embodiments, the γδ T cell is a δ3 T cell.

In some embodiments, there is provided a modified γδ T cell comprising a first heterologous nucleic acid sequence encoding galectin-9, and a second heterologous nucleic acid sequence encoding an engineered receptor (e.g., a CAR), wherein the γδ T cell expresses an MHC molecule. In some embodiments, the γδ T cell expresses an MHC class I molecule. In some embodiments, the γδ T cell expresses an MHC class II molecule. In some embodiments, the γδ T cell expresses both an MHC class I molecule and an MHC class II molecule. In some embodiments, the β2-microglobulin (B2M) gene of the γδ T cell is not genetically modified. In some embodiments, the MHC genes of the γδ T cell are not genetically modified. In some embodiments, the ICL compress an amino acid sequence having at least about 85% (e.g., about 90%, 95%, 98%, or more) sequence identity to the amino acid sequence of SEQ ID NO: 5. In some embodiments, the ICL comprises the amino acid sequence of SEQ ID NO: 5. In some embodiments, the engineered receptor targets a tumor antigen selected from the group consisting of CD19, BCMA, NY-ESO-1, VEGFR2, MAGE-A3, CD20, CD22, CD33, CD38, CEA, EGFR, GD2, HER2, IGF1R, mesothelin, PSMA, ROR1, WT1 and combinations thereof. In some embodiments, the engineered receptor is selected from the group consisting of a CAR, a TCR, a TAC receptor and a TFP. In some embodiments, the engineered receptor is a CAR. In some embodiments, the CAR is an anti-BCMA CAR. In some embodiments, the CAR is an anti-CD19 CAR. In some embodiments, the γδ T is a γ9δ2 T cell. In some embodiments, the γδ T cell is a δ1 T cell. In some embodiments, the γδ T cell is a δ3 T cell.

In some embodiments, there is provided a modified therapeutic cell comprising a first heterologous nucleic acid sequence encoding CD47, wherein the therapeutic cell expresses an MHC molecule. In some embodiments, the therapeutic cell expresses an MHC class I molecule. In some embodiments, the therapeutic cell expresses an MHC class II molecule. In some embodiments, the therapeutic cell expresses both an MHC class I molecule and an MHC class II molecule. In some embodiments, the β2-microglobulin (B2M) gene of the therapeutic cell is not genetically modified. In some embodiments, the MHC genes of the therapeutic cell are not genetically modified. In some embodiments, the ICL comprises an amino acid sequence having at least about 85% (e.g., about 90%, 95%, 98%, or more) sequence identity to the amino acid sequence of SEQ ID NO: 6. In some embodiments, the ICL comprises the amino acid sequence of SEQ ID NO: 6.

In some embodiments, there is provided a modified γδ T cell comprising a first heterologous nucleic acid sequence encoding CD47, wherein the γδ T cell expresses an MHC molecule. In some embodiments, the γδ T cell expresses an MHC class I molecule. In some embodiments, the γδ T cell expresses an MHC class II molecule. In some embodiments, the γδ T cell expresses both an MHC class I molecule and an MHC class II molecule. In some embodiments, the β2-microglobulin (B2M) gene of the γδ T cell is not genetically modified. In some embodiments, the MHC genes of the γδ T cell are not genetically modified. In some embodiments, the ICL comprises an amino acid sequence having at least about 85% (e.g., about 90%, 95%, 98%, or more) sequence identity to the amino acid sequence of SEQ ID NO: 6. In some embodiments, the ICL comprises the amino acid sequence of SEQ ID NO: 6. In some embodiments, the γδ T is a γ9δ2 T cell. In some embodiments, the γδ T cell is a δ1 T cell. In some embodiments, the γδ T cell is a δ3 T cell.

In some embodiments, there is provided a modified γδ T cell comprising a first heterologous nucleic acid sequence encoding CD47, and a second heterologous nucleic acid sequence encoding an engineered receptor (e.g., a CAR), wherein the γδ T cell expresses an MHC molecule. In some embodiments, the γδ T cell expresses an MHC class I molecule. In some embodiments, the γδ T cell expresses an MHC class II molecule. In some embodiments, the γδ T cell expresses both an MHC class I molecule and an MHC class II molecule. In some embodiments, the β2-microglobulin (B2M) gene of the γδ T cell is not genetically modified. In some embodiments, the MHC genes of the γδ T cell are not genetically modified. In some embodiments, the ICL comprises an amino acid sequence having at least about 85% (e.g., about 90%, 95%, 98%, or more) sequence identity to the amino acid sequence of SEQ ID NO: 6. In some embodiments, the ICL comprises the amino acid sequence of SEQ ID NO: 6. In some embodiments, the engineered receptor targets a tumor antigen selected from the group consisting of CD19, BCMA, NY-ESO-1, VEGFR2, MAGE-A3, CD20, CD22, CD33, CD38, CEA, EGFR, GD2, HER2, IGF1R, mesothelin, PSMA, ROR1, WT1 and combinations thereof. In some embodiments, the engineered receptor is selected from the group consisting of a CAR, a TCR, a TAC receptor and a TFP. In some embodiments, the engineered receptor is a CAR. In some embodiments, the CAR is an anti-BCMA CAR. In some embodiments, the CAR is an anti-CD19 CAR. In some embodiments, the γδ T is a γ9δ2 T cell. In some embodiments, the γδ T cell is a δ1 T cell. In some embodiments, the γδ T cell is a δ3 T cell.

In some embodiments, there is provided a modified therapeutic cell comprising a first heterologous nucleic acid sequence encoding B7H3, wherein the therapeutic cell expresses an MHC molecule. In some embodiments, the therapeutic cell expresses an MHC class I molecule. In some embodiments, the therapeutic cell expresses an MHC class II molecule. In some embodiments, the therapeutic cell expresses both an MHC class I molecule and an MHC class II molecule. In some embodiments, the β2-microglobulin (B2M) gene of the therapeutic cell is not genetically modified. In some embodiments, the MHC genes of the therapeutic cell are not genetically modified. In some embodiments, the ICL comprises an amino acid sequence having at least about 85% (e.g., about 90%, 95%, 98%, or more) sequence identity to the amino acid sequence of SEQ ID NO: 7. In some embodiments, the ICL comprises the amino acid sequence of SEQ ID NO: 7.

In some embodiments, there is provided a modified γδ T cell comprising a first heterologous nucleic acid sequence encoding B7H3, wherein the γδ T cell expresses an MHC molecule. In some embodiments, the γδ T cell expresses an MHC class I molecule. In some embodiments, the γδ T cell expresses an MHC class II molecule. In some embodiments, the γδ T cell expresses both an MHC class I molecule and an MHC class II molecule. In some embodiments, the β2-microglobulin (B2M) gene of the γδ T cell is not genetically modified. In some embodiments, the MHC genes of the γδ T cell are not genetically modified. In some embodiments, the ICL comprises an amino acid sequence having at least about 85% (e.g., about 90%, 95%, 98%, or more) sequence identity to the amino acid sequence of SEQ ID NO: 7. In some embodiments, the ICL comprises the amino acid sequence of SEQ ID NO: 7. In some embodiments, the γδ T is a γ9δ2 T cell. In some embodiments, the γδ T cell is a δ1 T cell. In some embodiments, the γδ T cell is a δ3 T cell.

In some embodiments, there is provided a modified γδ T cell comprising a first heterologous nucleic acid sequence encoding B7H3, and a second heterologous nucleic acid sequence encoding an engineered receptor (e.g., a CAR), wherein the γδ T cell expresses an MHC molecule. In some embodiments, the γδ T cell expresses an MHC class I molecule. In some embodiments, the γδ T cell expresses an MHC class II molecule. In some embodiments, the γδ T cell expresses both an MHC class I molecule and an MHC class II molecule. In some embodiments, the β2-microglobulin (B2M) gene of the γδ T cell is not genetically modified. In some embodiments, the MHC genes of the γδ T cell are not genetically modified. In some embodiments, the ICL comprises an amino acid sequence having at least about 85% (e.g., about 90° %, 95%, 98%, or more) sequence identity to the amino acid sequence of SEQ ID NO: 7. In some embodiments, the ICL comprises the amino acid sequence of SEQ ID NO: 7. In some embodiments, the engineered receptor targets a tumor antigen selected from the group consisting of CD19, BCMA, NY-ESO-1, VEGFR2, MAGE-A3, CD20, CD22, CD33, CD38, CEA, EGFR, GD2, HER2, IGF1R, mesothelin, PSMA, ROR1, WT1 and combinations thereof. In some embodiments, the engineered receptor is selected from the group consisting of a CAR, a TCR, a TAC receptor and a TFP. In some embodiments, the engineered receptor is a CAR. In some embodiments, the CAR is an anti-BCMA CAR. In some embodiments, the CAR is an anti-CD19 CAR. In some embodiments, the γδ T is a γ9δ2 T cell. In some embodiments, the γδ T cell is a δ1 T cell. In some embodiments, the γδ T cell is a δ3 T cell.

In some embodiments, there is provided a modified therapeutic cell comprising a first heterologous nucleic acid sequence encoding B7H4, wherein the therapeutic cell expresses an MHC molecule. In some embodiments, the therapeutic cell expresses an MHC class I molecule. In some embodiments, the therapeutic cell expresses an MHC class II molecule. In some embodiments, the therapeutic cell expresses both an MHC class I molecule and an MHC class II molecule. In some embodiments, the β2-microglobulin (B2M) gene of the therapeutic cell is not genetically modified. In some embodiments, the MHC genes of the therapeutic cell are not genetically modified. In some embodiments, the ICL comprises an amino acid sequence having at least about 85% (e.g., about 90%, 95%, 98%, or more) sequence identity to the amino acid sequence of SEQ ID NO: 8. In some embodiments, the ICL comprises the amino acid sequence of SEQ ID NO: 8.

In some embodiments, there is provided a modified γδ T cell comprising a first heterologous nucleic acid sequence encoding B7H4, wherein the γδ T cell expresses an MHC molecule. In some embodiments, the γδ T cell expresses an MHC class I molecule. In some embodiments, the γδ T cell expresses an MHC class II molecule. In some embodiments, the γδ T cell expresses both an MHC class I molecule and an MHC class II molecule. In some embodiments, the β2-microglobulin (B2M) gene of the γδ T cell is not genetically modified. In some embodiments, the MHC genes of the γδ T cell are not genetically modified. In some embodiments, the ICL comprises an amino acid sequence having at least about 85% (e.g., about 90%, 95%, 98%, or more) sequence identity to the amino acid sequence of SEQ ID NO: 8. In some embodiments, the ICL comprises the amino acid sequence of SEQ ID NO. 8. In some embodiments, the γδ T is a γ9δ2 T cell. In some embodiments, the γδ T cell is a β1 T cell. In some embodiments, the γδ T cell is a δ3 T cell.

In some embodiments, there is provided a modified γδ T cell comprising a first heterologous nucleic acid sequence encoding B7H4, and a second heterologous nucleic acid sequence encoding an engineered receptor (e.g., a CAR), wherein the γδ T cell expresses an MHC molecule. In some embodiments, the γδ T cell expresses an MHC class I molecule. In some embodiments, the γδ T cell expresses an MHC class II molecule. In some embodiments, the γδ T cell expresses both an MHC class I molecule and an MHC class II molecule. In some embodiments, the β2-microglobulin (B2M) gene of the γδ T cell is not genetically modified. In some embodiments, the MHC genes of the γδ T cell are not genetically modified. In some embodiments, the ICL comprises an amino acid sequence having at least about 85% (e.g., about 90%, 95%, 98%, or more) sequence identity to the amino acid sequence of SEQ ID NO: 8. In some embodiments, the ICL comprises the amino acid sequence of SEQ ID NO: 8. In some embodiments, the engineered receptor targets a tumor antigen selected from the group consisting of CD19, BCMA, NY-ESO-1, VEGFR2. MAGE-A3, CD20, CD22, CD33, CD38, CEA, EGFR, GD2, HER2, IGF1R, mesothelin, PSMA, ROR1, WT1 and combinations thereof. In some embodiments, the engineered receptor is selected from the group consisting of a CAR, a TCR, a TAC receptor and a TFP. In some embodiments, the engineered receptor is a CAR. In some embodiments, the CAR is an anti-BCMA CAR. In some embodiments, the CAR is an anti-CD19 CAR. In some embodiments, the γδ T is a γ9δ2 T cell. In some embodiments, the γδ T cell is a δ1 T cell. In some embodiments, the γδ T cell is a δ3 T cell.

The modified therapeutic cells can be derived from a variety of cell types and cell sources. Cells from any mammalian species, including, but not limited to, mice, rats, guinea pigs, rabbits, dogs, monkeys, and humans, are contemplated herein. In some embodiments, the therapeutic cell is a human cell.

The modified therapeutic cells may be used as adoptive cell therapies to treat a disease or condition of the individual, such as cancer, autoimmune disease, or infectious disease. Exemplary adoptive cell therapies, include, but are not limited to, tumor infiltrating lymphocytes (TIL), T cell receptor (TCR) modified T cells (TCR-Ts), chimeric antigen receptor (CAR) modified T cells, natural killer (NK) cells, NKT cells, and hematopoietic stem cells (HSCs), and dendritic cell (DC) or myeloid cell therapy. In some embodiments, the modified therapeutic cell is an αβ T cell. In some embodiments, the modified therapeutic cell is a γδ T cells. In some embodiments, the modified therapeutic cell is a γ9δ2 T cell. In some embodiments, the modified therapeutic cell is a δ1 T cell. In some embodiments, the modified therapeutic cell is a δ3 T cell.

In some embodiments, the therapeutic cell is autologous, i.e., the cell is derived from the individual who receives the therapeutic cells. In some embodiments, the therapeutic cell is syngeneic (i.e., the donor and the recipients are different individuals, but are identical twins, triplets or quadruplets, etc.). In some embodiments, the therapeutic cell is allogeneic, i.e., the cell is obtained or derived from a donor, who belongs to the same species, but is different from the individual receiving the therapeutic cells. In some embodiments, the allogeneic therapeutic cell is an off-the-shelf therapeutic cell, which is pre-manufactured, characterized, and made available for immediate administration to patients. In some embodiments, the allogeneic therapeutic cell is a “universal” therapeutic cell, which is derived from cells obtained from one or more donors or cell lines, and is used in adoptive cell therapy for other individuals of the same species.

In some embodiments, the therapeutic cell has genetic modifications that reduce immunogenicity or alloreactivity of the therapeutic cell. In some embodiments, the therapeutic cell is not genetically modified to reduce immunogenicity or alloreactivity of the therapeutic cell. In some embodiments, the therapeutic cell does not have genetic modification that affects its MHC complexes, including, for example, B2M mutation (e.g., knockout of B2M gene), or mutations to the MHC alleles. In some embodiments, the therapeutic cells do not elicit proliferation or cytotoxicity by NK cells. In some embodiments, compared to a therapeutic cell having MHC or B2M mutations (e.g., knockout), the modified therapeutic cell described herein has reduced recognition by host NK cells, for example, reduces proliferation or killing by NK cells by at least about any one of 10%, 20%, 30%, 40%, 50%, 60%, 70%, 80%, 90%, or more.

In some embodiments, the therapeutic cell is derived from a primary cell. In some embodiments, the therapeutic cell is a primary cell isolated from an individual. In some embodiments, the therapeutic cell is propagated (such as proliferated and/or differentiated) from a primary cell isolated from an individual. In some embodiments, the therapeutic cell is of the hematopoetic lineage. In some embodiments, the primary cell is obtained from the thymus. In some embodiments, the primary cell is obtained from the lymph or lymph nodes (such as tumor draining lymph nodes). In some embodiments, the primary cell is obtained from the spleen. In some embodiments, the primary cell is obtained from the bone marrow. In some embodiments, the primary cell is obtained from the blood, such as the peripheral blood. In some embodiments, the primary cell is a Peripheral Blood Mononuclear Cell (PBMC). In some embodiments, the primary cell is derived from the blood plasma. In some embodiments, the primary cell is derived from a tumor. In some embodiments, the primary cell is obtained from the mucosal immune system. In some embodiments, the primary cell is obtained from a biopsy sample. In some embodiments, the primary cell is obtained from cold blood.

In some embodiments, the therapeutic cell is derived from a cell line. In some embodiments, the therapeutic cell is obtained from a commercial cell line. In some embodiments, the therapeutic cell is propagated (such as proliferated and/or differentiated) from a cell line established from a primary cell isolated from an individual. In some embodiments, the cell line is mortal. In some embodiments, the cell line is immortalized. In some embodiments, the cell line is a tumor cell line, such as a leukemia or lymphoma cell line. In some embodiments, the cell line is a cell line derived from the PBMC. In some embodiments, the cell line is a stem cell line. In some embodiments, the cell line is NK-92.

In some embodiments, the therapeutic cell is an immune cell or a progenitor thereof. Exemplary immune cells useful for the present invention include, but are not limited to, dendritic cells (including immature dendritic cells and mature dendritic cells), T lymphocytes (such as naïve T cells, effector T cells, memory T cells, cytotoxic T lymphocytes, T helper cells, Natural Killer T cells, Treg cells, tumor infiltrating lymphocytes (TIL), and lymphokine-activated killer (LAK) cells), B cells, Natural Killer (NK) cells, γδ T cells (including, e.g., γ9δ2 T cells, δ1 T cells, δ3 T cells), αβ T cells, monocytes, macrophages, neutrophils, granulocytes, and combinations thereof. Subpopulations of immune cells can be defined by the presence or absence of one or more cell surface markers known in the art (e.g., CD3, CD4, CD8, CD19, CD20, CD11c, CD123, CD56, CD34, CD14, CD33, etc.). In the cases that the pharmaceutical composition comprises a plurality of modified therapeutic cells, the therapeutic cells can be a specific subpopulation of an immune cell type, a combination of subpopulations of an immune cell type, or a combination of two or more immune cell types. In some embodiments, the immune cell is present in a homogenous cell population. In some embodiments, the immune cell is present in a heterogeneous cell population that is enhanced in the immune cell. In some embodiments, the therapeutic cell is a lymphocyte. In some embodiments, the therapeutic cell is not a lymphocyte. In some embodiments, the therapeutic cell is suitable for adoptive cell therapy. In some embodiments, the therapeutic cell is a PBMC. In some embodiments, the therapeutic cell is an immune cell derived from a PBMC. In some embodiments, the therapeutic cell is a T cell. In some embodiment, the therapeutic cell is a CD4⁺ T cell (also known as helper T cell). In some embodiments, the therapeutic cell is a CD8⁺ T cell (also known as cytotoxic T cell). In some embodiments, the therapeutic cell is a T cell expressing TCRα and TCRβ chains (i.e., αβ T cell). In some embodiments, the therapeutic cell is a T cell expressing TCRγ and TCRδ chains (i.e., γδ T cell). In some embodiments, the therapeutic cell is a B cell. In some embodiments, the therapeutic cell is an NK cell. In some embodiments, the therapeutic cell is an NK-T cell. In some embodiments, the therapeutic cell is a dendritic cell (DC). In some embodiments, the therapeutic cell is a DC-activated T cell. In some embodiments, the modified immune cell is a γ9δ2 T cell. In some embodiments, the modified immune cell is a δ1 T cell. In some embodiments, the modified immune cell is a δ3 T cell.

In some embodiments, there is provided a modified immune cell comprising a first heterologous nucleic acid sequence encoding an ICL selected from the group consisting of PD-L1, CD155, CD112, FGL1, galectin-9, CD47, B7H3 and B7H4, wherein the immune cell expresses an MHC molecule. In some embodiments, the immune cell expresses an MHC class I molecule. In some embodiments, the immune cell expresses an MHC class II molecule. In some embodiments, the immune cell expresses both an MHC class I molecule and an MHC class II molecule. In some embodiments, the β2-microglobulin (B2M) gene of the immune cell is not genetically modified. In some embodiments, the MHC genes of the immune cell are not genetically modified. In some embodiments, the immune cell is selected from the group consisting of cytotoxic T cell, helper T cell, NK cell, NK-T cell, αβ T cell, γδ T cell, tumor-infiltrating T cell, dendritic cell (DC)-activated T cell, and peripheral blood mononuclear cell (PBMC). In some embodiments, the modified therapeutic cell is a T cell. In some embodiments, the modified therapeutic cell is a γδ T cell.

In some embodiments, the modified therapeutic cell is a stem cell or derived from a stem cell. In some embodiments, the stem cell is a totipotent stem cell. In some embodiments, the stem cell is a pluripotent stem cell. In some embodiments, the stem cell is a unipotent stem cell. In some embodiments, the stem cell is a progenitor cell. In some embodiments, the stem cell is an embryonic stem cell (ESC). In some embodiments, the stem cell is hematopoietic stem cell (HSC). In some embodiments, the stem cell is a mesenchymal stem cell. In some embodiments, the stem cell is an induced pluripotent stem cell (iPSC).

In some embodiments, there is provided a modified stem cell comprising a first heterologous nucleic acid sequence encoding an ICL selected from the group consisting of PD-L1, CD155, CD112, FGL1, galectin-9, CD47, B7H3 and B7H4, wherein the stem cell expresses an MHC molecule. In some embodiments, the stem cell expresses an MHC class I molecule. In some embodiments, the stem cell expresses an MHC class II molecule. In some embodiments, the stem cell expresses both an MHC class I molecule and an MHC class II molecule. In some embodiments, the β2-microglobulin (B2M) gene of the stem cell is not genetically modified. In some embodiments, the MHC genes of the stem cell are not genetically modified. In some embodiments, the modified stem cell is an HSC. In some embodiments, the modified stem cell is an ESC.

In some embodiments, the modified therapeutic cell comprises one or more heterologous nucleic acid sequences. The modified immune cell may comprise any number (such as any of 1, 2, 3, 4, 5, 10, 50, 100, 1000, or more) of the heterologous nucleic acid sequence(s). In some embodiments, the modified immune Cell comprises a single copy of the heterologous nucleic acid sequence. In some embodiments, the modified immune cell comprises a plurality of copies of the heterologous nucleic acid sequence(s).

In some embodiments, the modified therapeutic cell (e.g., immune cell or stem cell) further comprises a second heterologous nucleic acid sequence encoding an engineered receptor. In some embodiments, the modified therapeutic cell expresses two or more engineered receptors. In some embodiments, the modified therapeutic cell expresses one or more engineered receptors selected from the group consisting of a chimeric antigen receptor (CAR), a recombinant T cell receptor (TCR), a T-cell antigen coupler (TAC) receptor and a TCR fusion protein (TFP). In some embodiments, the therapeutic cell is a CAR-T cell. In some embodiments, the therapeutic cell is an allogeneic CAR-T cell. In some embodiments, the therapeutic cell is a UCAR-T cell. In some embodiments, the therapeutic cell is a TCR-T cell. In some embodiments, the modified immune cells express a CAR and a TFP. In some embodiments, the modified immune cells express a CAR and a recombinant TCR. In some embodiments, the modified immune cells express a CAR and a TAC receptor. In some embodiments, the modified immune cells express a recombinant TCR and a TAC receptor. In some embodiments, the modified immune cell further comprises at least one additional heterologous nucleic acid sequence, for example, a third heterologous nucleic acid sequence encoding an immunomodulatory agent, such as a co-stimulatory molecule, a cytokine, and/or a chemokine. In some embodiments, the therapeutic cell does not have genetic modification other than the first and second heterologous nucleic acid sequences.

In some embodiments, there is provided a modified immune cell comprising a first heterologous nucleic acid sequence encoding an ICL selected from the group consisting of PD-L1, CD155, CD112, FGL1, galectin-9, CD47, B7H3 and B7H4, and a second heterologous nucleic acid sequence encoding an engineered receptor, wherein the immune cell expresses an MHC molecule. In some embodiments, the immune cell expresses an MHC class I molecule. In some embodiments, the immune cell expresses an MHC class II molecule. In some embodiments, the immune cell expresses both an MHC class I molecule and an MHC class II molecule. In some embodiments, the β2-microglobulin (B2M) gene of the immune cell is not genetically modified. In some embodiments, the MHC genes of the immune cell are not genetically modified. In some embodiments, the engineered receptor is selected from the group consisting of a chimeric antigen receptor (CAR), a recombinant T cell receptor (TCR), a T-cell antigen coupler (TAC) receptor and a TCR fusion protein (TFP). In some embodiments, the engineered receptor targets a tumor antigen selected from the group consisting of CD19, BCMA, NY-ESO-1, VEGFR2, MAGE-A3, CD20, CD22, CD33, CD38, CEA, EGFR, GD2, HER2, IGF1R, mesothelin, PSMA, ROR1, WT1 and combinations thereof.

In some embodiments, there is provided a modified CAR-T cell comprising a first heterologous nucleic acid sequence encoding an ICL selected from the group consisting of PD-L1, CD1S5, CD112, FGL1, galectin-9, CD47, B7H3 and B7H4, and a second heterologous nucleic acid sequence encoding a CAR, wherein the CAR-T cell expresses an MIC molecule. In some embodiments, the CAR-T cell expresses an MHC class I molecule. In some embodiments, the CAR-T cell expresses an MHC class II molecule. In some embodiments, the CAR-T cell expresses both an MHC class I molecule and an MHC class II molecule. In some embodiments, the β2-microglobulin (B2M) gene of the CAR-T cell is not genetically modified. In some embodiments, the MHC genes of the CAR-T cell are not genetically modified. In some embodiments, the CAR targets a tumor antigen selected from the group consisting of CD19, BCMA, NY-ESO-1, VEGFR2, MAGE-A3, CD20, CD22, CD33, CD38, CEA, EGFR, GD2, HER2, IGF1R, mesothelin, PSMA, ROR1, WT1 and combinations thereof. In some embodiments, the CAR is an anti-BCMA CAR. In some embodiments, the CAR is an anti-CD19 CAR.

In some embodiments, there is provided a modified γδ T cell comprising a first heterologous nucleic acid sequence encoding an ICL selected from the group consisting of PD-L1, CD5S, CD112, FGL1, galectin-9, CD47, B7H3 and B7H4, wherein the γδ T cell expresses an MHC molecule. In some embodiments, the γδ T cell expresses an MHC class I molecule. In some embodiments, the γδ T cell expresses an MHC class II molecule. In some embodiments, the γδ T cell expresses both an MHC class I molecule and an MHC class II molecule. In some embodiments, the β2-microglobulin (B2M) gene of the γδ T cell is not genetically modified. In some embodiments, the MHC genes of the γδ T cell are not genetically modified. In some embodiments, the γδ T is a γ9δ2 T cell. In some embodiments, the γδ T cell is a δ1 T cell. In some embodiments, the γδ T cell is a δ3 T cell.

In some embodiments, there is provided a modified γδ T cell comprising a first heterologous nucleic acid sequence encoding an ICL selected from the group consisting of PD-L1, CD155, CD112, FGL1, galectin-9, CD47, B7H3 and B7H4, and a second heterologous nucleic acid sequence encoding an engineered receptor (e.g., a CAR), wherein the γδ T cell expresses an MHC molecule. In some embodiments, the γδ T cell expresses an MHC class I molecule. In some embodiments, the γδ T cell expresses an MHC class II molecule. In some embodiments, the γδ T cell expresses both an MHC class I molecule and an MHC class II molecule. In some embodiments, the β2-microglobulin (B2M) gene of the γδ T cell is not genetically modified. In some embodiments, the MHC genes of the γδ T cell are not genetically modified. In some embodiments, the engineered receptor targets a tumor antigen selected from the group consisting of CD19, BCMA, NY-ESO-1, VEGFR2, MAGE-A3, CD20, CD22, CD33, CD38, CEA, EGFR, GD2, HER2, IGF1R, mesothelin, PSMA, ROR1, WT1 and combinations thereof. In some embodiments, the engineered receptor is selected from the group consisting of a chimeric antigen receptor (CAR), a recombinant T cell receptor (TCR), a T-cell antigen coupler (TAC) receptor and a TCR fusion protein (TFP). In some embodiments, the engineered receptor is a CAR. In some embodiments, the CAR is an anti-BCMA CAR. In some embodiments, the CAR is an anti-CD19 CAR. In some embodiments, the γδ T is a γ9δ2 T cell. In some embodiments, the γδ T cell is a δ1 T cell. In some embodiments, the γδ T cell is a δ3 T cell.

In some embodiments, the modified therapeutic cell does not have any genetic modification, e.g., genomic modification, to reduce immunogenicity of the therapeutic cell in the individual. In some embodiments, the modified therapeutic cell has one or more genetic modifications, e.g., genomic modifications, to reduce immunogenicity of the therapeutic cell in the individual. In some embodiments, the genetic modification comprises genetically disrupting the TCR gene and/or HLA class I loci of allogeneic T cells. In some embodiments, the genetic modification comprises knocking out of endogenous TCR genes, e.g., TRAC (i.e., TCRα), TRBC (i.e., TCRβ), TCRG (i.e., TCRγ), and/or TCRD (i.e., TCRδ) genes. In some embodiments, the genetic modification comprises knocking out of β2-microglobulin (B2M). In some embodiments, the genetic modification comprises knocking out of an immune checkpoint molecule, such as PD-1 or CTLA-4 (also known as CD52).

In some embodiments, the modified therapeutic cell is a T cell, such as allogeneic T cell. In some embodiments, the therapeutic cell is a TCRαβ⁺ T cell. In some embodiments, the therapeutic cell is a TCRγδ⁺ T cell. In some embodiments, the therapeutic cell is a CAR-T cell. In some embodiments, the therapeutic cell expresses an anti-BCMA CAR. In some embodiments, the therapeutic cell is a CAR-T cell expressing a BSF17 CAR. In some embodiments, the therapeutic cell expresses an anti-CD19 CAR. In some embodiments, the therapeutic cell is a CAR-T cell expressing a CTL-019 CAR. In some embodiments, the therapeutic cell is a TCR-T cell. In some embodiments, the therapeutic cell is a T cell expressing a TAC receptor. In some embodiments, the therapeutic cell is a T cell expressing a TFP. In some embodiments, the therapeutic cell is a T cell expressing a combination of engineered receptors selected from the group consisting of CAR, TCR, TAC receptor and TFP. In some embodiments, the therapeutic T cell comprises an endogenous TCR. In some embodiments, the therapeutic T cell does not have genetic modifications that reduce their immunogenicity. In some embodiments, the endogenous TCR genes, HLA genes (e.g., B2M), and immune checkpoint molecules (e.g., PD-1, CTLA-4, etc.) of the therapeutic T cells are not genetically modified. In some embodiments, the therapeutic T cell has no genetic modification except for the ICL and the engineered receptor construct. In some embodiments, the therapeutic cell is a CAR-T cell expressing a BSF17 CAR and PD-L1. In some embodiments, the therapeutic cell is a CAR-T cell expressing a CTL-019 CAR and PD-II.

In some embodiments, the therapeutic cell is a HSC, such as an allogeneic HSC. In some embodiments, the therapeutic cell is an HSC that has no genetic modifications other than the ICL. In some embodiments, the therapeutic cell is an HSC that expresses one or more therapeutic agents. In some embodiments, the therapeutic cell is an HSC that expresses one or more engineered receptors selected from the group consisting of CAR, TCR, TAC receptor and TFP. In some embodiments, the therapeutic cell is an HSC expressing one or more therapeutic agents other than engineered receptors. In some embodiments, the therapeutic cell is a viral vector-transduced HSC, such as retroviral or lentiviral transduced HSC.

In some embodiments, the therapeutic cell is an NK cell. In some embodiments, the therapeutic cell is an NK cell expressing one or more engineered receptors selected from the group consisting of CAR, TCR, TAC receptor and TFP.

In some embodiments, the therapeutic cell is an NK-T cell. In some embodiments, the therapeutic cell is an NK-T cell expressing one or more engineered receptors selected from the group consisting of CAR, TCR, TAC receptor and TFP.

In some embodiments, expression of an ICL (e.g. PD-L1) by a therapeutic cell (e.g. CAR-T cell) does not reduce cytotoxicity of the therapeutic cell, for example, by any one of 50%, 40%, 30%, 20%, 10%, or less, compared to a therapeutic cell that does not express the ICL. In some embodiments, expression of an ICL (e.g. PD-L1) by a therapeutic cell (e.g. CAR-T cell) does not reduce long-term persistence of the therapeutic cell, for example, by any one of 50%, 40%, 30%, 20%, 10%, or less, compared to a therapeutic cell that does not express the ICL. In some embodiments, expression of an ICL (e.g. PD-L1) by a therapeutic cell (e.g. CAR-T cell) does not alter (e.g., increase or decrease) cytokine production profile of the therapeutic cell, for example, by any one of 50%, 40%, 30%, 20%, 10%, or less, compared to a therapeutic cell that does not express the ICL. In some embodiments, expression of an ICL (e.g. PD-L1) by a therapeutic cell (e.g. CAR-T cell) does not alter (e.g., increase or decrease) anti-tumor efficacy of the therapeutic cell, for example, by any one of 50%, 40%, 30%, 20%, 10%, or less, compared to a therapeutic cell that does not express the ICL. In some embodiments, expression of an ICL (e.g. PD-L1) by a therapeutic cell (e.g. CAR-T cell) does not alter (e.g., increase or decrease) proliferation of the therapeutic cell, for example, by any one of 50%, 40%, 30%, 20%, 10%, or less, compared to a therapeutic cell that does not express the ICL. In some embodiments, any one of the effects of ICL expression described in this paragraph is assessed in vitro. In some embodiments, any one of the effects of ICL expression described in this paragraph is assessed in vivo.

In some embodiments, expression of an ICL (e.g., PD-L1) by a therapeutic cell (e.g., CAR-T cell) reduces proliferation of allogeneic immune cells (e.g., αβT cell and/or NK cells) of a subject receiving the therapeutic cell, for example, by at least about any one of 10%, 20%, 30%, 40%, 50%, 60%, 70%, 80%, 90%, or more, compared to a therapeutic cell that does not express the ICL. In some embodiments, expression of an ICL (e.g., PD-L1) by a therapeutic cell (e.g., CAR-T cell) reduces proliferation of allogeneic αβT cells of a subject receiving the therapeutic cell, for example, by at least about any one of 10%, 20%, 30%, 40%, 50%, 60%, 70%, 80%, 90%, or more, compared to a therapeutic cell that does not express the ICL. In some embodiments, expression of an ICL (e.g., PD-L1) by a therapeutic cell (e.g., CAR-T cell) reduces proliferation of allogeneic NK cells of a subject receiving the therapeutic cell, for example, by at least about any one of 10%, 20%, 30%, 40%, 50%, 60%, 70%, 80%, 90%, or more, compared to a therapeutic cell that does not express the ICL

Nucleic acid(s) comprising the heterologous nucleic acid sequence(s) described herein may be transiently or stably incorporated in the modified therapeutic cells. In some embodiments, the nucleic acid(s) is transiently expressed in the modified therapeutic cell. For example, the nucleic acid(s) may be present in the nucleus of the modified therapeutic cell in an extrachromosomal array. The nucleic acid(s) may be introduced into the modified therapeutic cell using any transfection or transduction methods known in the art, including viral or non-viral methods. Exemplary non-viral transfection methods include, but are not limited to, chemical-based transfection, such as using calcium phosphate, dendrimers, liposomes, or cationic polymers (e.g., DEAE-dextran or polyethylenimine); non-chemical methods, such as electroporation, cell squeezing, sonoporation, optical transfection, impalefection, protoplast fusion, hydrodynamic delivery, or transposons; particle-based methods, such as using a gene gun, magnetofection or magnet assisted transfection, particle bombardment; and hybrid methods, such as nucleofection.

In some embodiments, the heterologous nucleic acid sequence(s) is present in the genome of the modified therapeutic cell. For example, nucleic acid(s) comprising the heterologous nucleic acid sequence(s) may be integrated into the genome of the modified therapeutic cell by any methods known in the art, including, but not limited to, virus-mediated integration, random integration, homologous recombination methods, and site-directed integration methods, such as using site-specific recombinase or integrase, transposase, Transcription activator-like effector nuclease (TALEN®), CRISPR/Cas9, and zinc-finger nucleases. In some embodiments, the heterologous nucleic acid sequence(s) is integrated in a specifically designed locus of the genome of the modified therapeutic cell. In some embodiments, the heterologous nucleic acid sequence(s) is integrated in an integration hotspot of the genome of the modified therapeutic cell. In some embodiments, the heterologous nucleic acid sequence(s) is integrated in a random locus of the genome of the modified therapeutic cell. In the cases that multiple copies of the heterologous nucleic acid sequence(s) are present in a single modified therapeutic cell, the heterologous nucleic acid sequences may be integrated in a plurality of loci of the genome of the modified therapeutic cell.

Precursor immune cells can be prepared using a variety of methods known in the art. For example, primary immune cells, such as T cells can be obtained from a number of sources, including peripheral blood mononuclear cells, bone marrow, lymph node tissue, cord blood, thymus tissue, tissue from a site of infection, ascites, pleural effusion, spleen tissue, and tumors. In some embodiments, immune cells (such as T cells) can be obtained from a unit of blood collected from an individual using any number of techniques known in the art, such as FICOLL™ separation. In some embodiments, cells from the circulating blood of an individual are obtained by apheresis. The apheresis product typically contains lymphocytes, including T cells, monocytes, granulocytes, B cells, other nucleated white blood cells, red blood cells, and platelets. In some embodiments, the cells collected by apheresis may be washed to remove the plasma fraction and to place the cells in an appropriate buffer or media for subsequent processing steps. In some embodiments, the cells are washed with phosphate buffered saline (PBS), or a wash solution lacking divalent cations, such as calcium and magnesium. As those of ordinary skill in the art would readily appreciate a washing step may be accomplished by methods known to those in the art, such as by using a semi-automated “flow-through” centrifuge (for example, the Cobe 2991 cell processor, the Baxter CytoMate, or the Haemonetics Cell Saver 5) according to the manufacturer's instructions. After washing, the cells may be resuspended in a variety of biocompatible buffers, such as, for example, Ca²⁺-free, Mg²⁺-free PBS, PlasmaLyte A, or other saline solution with or without buffer. Alternatively, the undesirable components of the apheresis sample may be removed and the cells directly resuspended in culture media.

In some embodiments, primary T cells are isolated from peripheral blood lymphocytes by lysing the red blood cells and depleting the monocytes, for example, by centrifugation through a PERCOLL™ gradient or by counterflow centrifugal elutriation. A specific subpopulation of T cells, such as CD3⁺, CD28⁻, CD4⁺, CD8⁺, CD45RA, and CD45RO cells, can be further isolated by positive or negative selection techniques. For example, in one embodiment, T cells are isolated by incubation with anti-CD3/anti-CD28 (i.e., 3×28)-conjugated beads, such as DYNABEADS® M-450 CD3/CD28 T, for a time period sufficient for positive selection of the desired T cells.

In some embodiments, a T cell population may further be enriched by negative selection using a combination of antibodies directed to surface markers unique to the negatively selected cells. For example, one method involves cell sorting and/or selection via negative magnetic immunoadherence or flow cytometry that uses a cocktail of monoclonal antibodies directed to cell surface markers present on the cells negatively selected. For example, to enrich for CD4⁺ cells by negative selection, a monoclonal antibody cocktail typically includes antibodies to CD14, CD20, CD11b, CD16, HLA-DR, and CD8. In certain embodiments, it may be desirable to enrich for or positively select for regulatory T cells, which typically express CD4⁺, CD25⁺, CD62L^{hi}, GITR⁺, and FoxP3⁺.

Methods of introducing vectors or nucleic acids into a therapeutic cell (such as a precursor immune cell) are known in the art. The vectors or nucleic acids can be transferred into a therapeutic cell by physical, chemical, or biological methods.

Physical methods for introducing the vector(s) or nucleic acid(s) into a therapeutic cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2001) Molecular Cloning: A Laboratory Manual, Cold Spring Harbor Laboratory, New York. In some embodiments, the vector is introduced into the cell by electroporation.

Biological methods for introducing the vector(s) or nucleic acid(s) into a therapeutic cell include the use of DNA and RNA vectors. Viral vectors have become the most widely used method for inserting genes into mammalian, e.g., human cells.

Chemical means for introducing the vector(s) or nucleic acid(s) into a therapeutic cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro is a liposome (e.g., an artificial membrane vesicle).

In some embodiments, the transduced or transfected precursor immune cell is propagated ex vivo after introduction of the heterologous nucleic acid(s). In some embodiments, the transduced or transfected precursor immune cell is cultured to propagate for at least about any of 1 day, 2 days, 3 days, 4 days, 5 days, 6 days, 7 days, 10 days, 12 days, or 14 days. In some embodiments, the transduced or transfected precursor immune cell is cultured for no more than about any of 1 day, 2 days, 3 days, 4 days, 5 days, 6 days, 7 days, 10 days, 12 days, or 14 days. In some embodiments, the transduced or transfected precursor immune cell is further evaluated or screened to select the modified immune cell.

Reporter genes may be used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al. FEBS Letters 479: 79-82 (2000)).

Other methods to confirm the presence of the heterologous nucleic acid(s) in the precursor immune cell, include, for example, molecular biological assays well known to those of skill in the an, such as Southern and Northern blotting, RT-PCR and PCR; biochemical assays, such as detecting the presence or absence of a particular peptide, e.g., by immunological methods (such as ELISAs and Western blots).

### A. Immune Checkpoint Ligands

The modified therapeutic cells described herein overexpress one or more immune checkpoint ligands (ICL), including, but not limited to PD-L1, CD155, CD112, FGL1, galectin-9, CD47, B7H3 and B7H4.

Tumor cells escape detection and elimination by the immune system through multiple mechanisms. Immune checkpoint pathways are used in maintenance of self-tolerance and control of T cell activation, but cancer cells can use the pathways to suppress the anti-tumor response and prevent their destruction. Without being bound by any theory or hypothesis, graft rejection is mediated by patient T and NK cells. Central to adaptive immunity is the interaction between the αβ T cell antigen receptor (TCR) on T cells and peptides presented by the major histocompatibility complex (MHC) molecules (including MHC class I and class II molecules) on target cells. NK cells can recognize target cells that do not express MHC molecules. Allogeneic cells may cause graft rejection when administered to an MHC-mismatched patient. The patient's own T cells and NK cells could reject the infused therapeutic cells, resulting in diminished therapeutic efficacy. See, for example, Dao M et al., Cancer Res (2018) 78: 3588; Patel S et al. Front Oncol. (2019); 9: 196. Activated immune cells express immune checkpoint proteins (ICP), such as PD-1, TIM-3, LAG-3, TIGIT and thrombospondin-1. When ICP ligands are overexpressed on immune cells, ICP ligand can bind to the ICP on immune cells and cause immune cell dysfunction.

### PD-L1

In some embodiments, the ICL is programmed cell death ligand 1 (PD-L1). In some embodiments, the ICL is human PD-L1. Sequences of PID-LA molecules are known in the art. See, for example, GenBank ID AAI13735.1 and UniProtKB ID Q9NZQ7. PD-L1 is also known as CD274, B7-H1, and PDCD1 ligand 1 (PDCD1L1 or PDCD1LG1). In some embodiments, the human PD-L1 has the amino acid sequence of SEQ ID NO: 1 shown below. In some embodiments, the ICL comprises an amino acid sequence having at least about 85% (e.g., at least about any one of 86%, 87%, 88%, 89%, 90%, 91%, 92%, 93%, 94%, 95%, 96%, 97%, 98%, 99%, or more) sequence identity to SEQ ID NO: 1. In some embodiments, the ICL is a functional fragment of a naturally occurring PD-L1. In some embodiments, the ICL is a full-length naturally occurring PD-L1. In some embodiment, the ICL comprises a signal peptide. In some embodiments, the ICL does not comprise a signal peptide. In some embodiments, the ICL comprises the extracellular domain of a naturally occurring PD-L1. In some embodiments, the ICL comprises the extracellular domain, transmembrane domain and cytoplasmic domain of a naturally occurring PD-L1. In some embodiments, the ICL comprises an amino acid sequence comprising amino acids 19-290 of SEQ ID NO: 1, or an amino acid sequence having at least about 85% (e.g., at least any one of 86%, 87%, 88%, 89%, 90%, 91%, 92%, 93%, 94%, 95%, 96%, 97%, 98%, 99%, or more) sequence identity to amino acids 19-290 of SEQ ID NO: 1.

(full length human PD-L1 amino acid sequence;
signal peptide is bolded)
SEQ ID NO: 1
MRIFAVFIFMTYWHLLNAFTVTVPKDLYVVEYGSNMTIECKFPVEKQL
DLAALIVYWEMEDKNIIQFVHGEEDLKVQHSSYRQRARLLKDQLSLGN
AALQITDVKLQDAGVYRCMISYGGADYKRITVKVNAPYNKINQRILVV
DPVTSEHELTCQAEGYPKAEVIWTSSDHQVLSGKTTTTNSKREEKLFN
VTSTLRINTTTNEIFYCTFRRLDPEENHTAELVIPELPLAHPPNERTH
LVILGAILLCLGVALTFIFRLRKGRMMDVKKCGIQDTNSKKQSDTHLE
ET

The PD-L1/human programmed cell death 1 (PD-1) pathway is an immune checkpoint that regulates self-tolerance and T cell activation. Human PD-1 is found on T cells, NK cells and other immune cells. Binding of PD-L1 to PD-1 inhibits T cell and NK cell proliferation and cytokine production. The PD-1/PD-L1 inhibitory axis has been subjugated by tumors as part of the natural selective process that shapes tumor evolution in the context of an anti-tumor immune response. PD-L1 also binds to B7-1 (CD80). B7-1 is another negative regulator of T cell and NK cell activation. Accordingly, PD-L1 is aberrantly expressed by a variety of tumor types, and increased expression of PD-L1 on tumor cells correlates with worse prognosis in many types of cancer. PD-L1 expression is also up-regulated in the tumor microenvironment in immune cells and other cell types as a result of immune activation and production of pro-inflammatory cytokines, which further contributes to the establishment of an immunosuppressive environment.

### CD155

In some embodiments, the ICL is CD155. In some embodiments, the ICL is human CD155. Sequences of CD155 molecules are known in the art. See, for example, GenBank ID NP_006496.4 and UniProtKB ID P15151. CD155 is also known as poliovirus receptor (PVR) or nectin-like protein 5 (NECL-5). In some embodiments, the human CD155 has the amino acid sequence of SEQ ID NO: 2 shown below. In some embodiments, the ICL comprises an amino acid sequence having at least about 85% (e.g., at least about any one of 86%, 87%, 88%, 89%, 90%, 91%, 92%, 93%, 94%, 95%, 96%, 97%, 98%, 99%, or more) sequence identity to SEQ ID NO: 2. In some embodiments, the ICL is a functional fragment of a naturally occurring CD155. In some embodiments, the ICL is a full-length naturally occurring CD155. In some embodiment, the ICL comprises a signal peptide. In some embodiments, the ICL does not comprise a signal peptide. In some embodiments, the ICL comprises the extracellular domain of a naturally occurring CD155. In some embodiments, the ICL comprises the extracellular domain, transmembrane domain and cytoplasmic domain of a naturally occurring CD155. In some embodiments, the ICL comprises an amino acid sequence comprising amino acids 21-417 of SEQ ID NO: 2, or an amino acid sequence having at least about 85% (e.g., at least about any one of 86%, 87%, 88%, 89%, 90%, 91%, 92%, 93%, 94%, 95%, 96%, 97%, 98%, 99%, or more) sequence identity to amino acids 21-417 of SEQ ID NO: 2.

(full length human CD155 amino acid sequence;
signal peptide is bolded)
SEQ ID NO: 2
MARAMAAAWPLLLVALLVLSWPPPGTGDVVVQAPTQVPGFLGDSVTLP
CYLQVPNMEVTHVSQLTWARHGESGSMAVFHQTQGPSYSESKRLEFVA
ARLGAELRNASLRMFGLRVEDEGNYTCLFVTFPQGSRSVDIWLRVLAK
PQNTAEVQKVQLTGEPVPMARCVSTGGRPPAQITWHSDLGGMPNTSQV
PGFLSGTVTVTSLWILVPSSQVDGKNVTCKVEHESFEKPQLLTVNLTV
YYPPEVSISGYDNNWYLGQNEATLTCDARSNPEPTGYNWSTTMGPLPP
FAVAQGAQLLIRPVDKPINTTLICNVTNALGARQAELTVQVKEGPPSE
HSGISRNAIIFLVLGILVFLILLGIGIYFYWSKCSREVLWHCHLCPSS
TEHASASANGHVSYSAVSRENSSSQDPQTEGTR

Poliovirus receptor (PVR)-like proteins are a newly identified group of the immunoglobulin superfamily (IGSF) with T cell co-signaling functions. This group of molecules share a PVR signature motif in the first Ig variable-like (IgV) domain, and are originally known to mediate epithelial cell-cell contacts. CD155 (PVR) was identified as the counter structure for TIGIT (T Cell Immunoreceptor With Ig And ITIM Domains). CD155 has been reported as the counter structure for at least two other receptors including CD226 (DNAM-1) and CD96 (Tactile). CD226 and CD96 are activating receptors expressed on T cells and NK cells, and CD155 can trigger T cell and NK cell activation through these receptors. CD155 is widely expressed in non-hematopoietic tissues and may be overexpressed in a large number of tumors and transformed cell types. CD155 engages TIGIT to suppress T cell and NK cell responses against the tumor cells.

### CD112

In some embodiments, the ICL is CD112. In some embodiments, the ICL is human CD112. Sequences of CD112 molecules are known in the art. See, for example, GenBank ID NP_002847.1 and UniProtKB ID Q92692. CD112 is also known as nectin 2, herpes virus entry mediator B (HveB), nectin cell adhesion molecule 2 (NECTIN2), poliovirus receptor-related protein 2 (PRR2 or PVRL2). In some embodiments, the human CD112 has the amino acid sequence of SEQ ID NO: 3 shown below. In some embodiments, the ICL comprises an amino acid sequence having at least about 85% (e.g., at least about any one of 86%, 87%, 88%, 89%, 90%, 91%, 92%, 93%, 94%, 95%, 96%, 97%, 98%, 99%, or more) sequence identity to SEQ ID NO: 3. In some embodiments, the ICL is a functional fragment of a naturally occurring CD112. In some embodiments, the ICL is a full-length naturally occurring CD112. In some embodiment, the ICL comprises a signal peptide. In some embodiments, the ICL does not comprise a signal peptide. In some embodiments, the ICL comprises the extracellular domain of a naturally occurring CD112. In some embodiments, the ICL comprises the extracellular domain, transmembrane domain and cytoplasmic domain of a naturally occurring CD112. In some embodiments, the ICL comprises an amino acid sequence comprising amino acids 32-538 of SEQ ID NO: 3, or an amino acid sequence having at least about 85% (e.g., at least about any one of 86%, 87%, 88%, 89%, 90%, 91%, 92%, 93%, 94%, 95%, 96%, 97%, 98%, 99%, or more) sequence identity to amino acids 32-538 of SEQ ID NO: 3.

(full length human CD112 amino acid sequence;
signal peptide is bolded)
SEQ ID NO: 3
MARAAALLPSRSPPTPLLWPLLLLLLLETGAQDVRVQVLPEVRGQLGG
TVELPCHLLPPVPGLYISLVTWQRPDAPANHQNVAAFHPKMGPSFPSP
KPGSERLSFVSAKQSTGQDTEAELQDATLALHGLTVEDEGNYTCEFAT
FPKGSVRGMTWLRVIAKPKNQAEAQKVTFSQDPTTVALCISKEGRPPA
RISWLSSLDWEAKETQVSGTLAGTVTVTSRFTLVPSGRADGVTVTCKV
EHESFEEPALIPVTLSVRYPPEVSISGYDDNWYLGRTDATLSCDVRSN
PEPTGYDWSTTSGTFPTSAVAQGSQLVIHAVDSLFNTTFVCTVTNAVG
MGRAEQVIFVRETPNTAGAGATGGIIGGIIAAIIATAVAATGILICRQ
QRKEQTLQGAEEDEDLEGPPSYKPPTPKAKLEAQEMPSQLFTLGASEH
SPLKTPYFDAGASCTEQEMPRYHELPTLEERSGPLHPGATSLGSPIPV
PPGPPAVEDVSLDLEDEEGEEEEEYLDKINPIYDALSYSSPSDSYQGK
GFVMSRAMYV

CD112 is a modulator of T cell and NK cell signaling. CD112 (PVRL2/nectin-2) interacts with CD226 (DNAM-1) to co-stimulate T cells and NK cells. CD112 may also inhibit T cell response through binding to another co-inhibitory receptor, TIGIT.

In some embodiments, the ICL is fibrinogen-like protein 1 (FGL1). In some embodiments, the ICL is human FGL1. Sequences of FGL1 molecules are known in the art. See, for example, GenBank ID NP_963846.1 and UniProtKB ID Q08830. FGL1 is also known as HP-041, Hepassocin (HPS), hepatocyte-derived fibrinogen-related protein 1 (HFREP-1), or liver fibrinogen-related protein 1 (LFIRE-1). In some embodiments, the human FGL1 has the amino acid sequence of SEQ ID NO: 4 shown below. In some embodiments, the ICL comprises an amino acid sequence having at least about 85% (e.g., at least about any one of 86%, 87%, 88%, 89%, 90%, 91%, 92%, 93%, 94%, 95%, 96%, 97%, 98%, 99%, or more) sequence identity to SEQ ID NO: 4. In some embodiments, the ICL is a functional fragment of a naturally occurring FGL1. In some embodiments, the ICL is a full-length naturally occurring FGL1. In some embodiment, the ICL comprises a signal peptide. In some embodiments, the ICL does not comprise a signal peptide. In some embodiments, the ICL comprises an amino acid sequence comprising amino acids 23-312 of SEQ ID NO: 4, or an amino acid sequence having at least about 85% (e.g., at least about any one of 86%, 87%, 88%, 89%, 90%, 91%, 92%, 93%, 94%, 95%, 96%, 97%, 98%, 99%, or more) sequence identity to amino acids 23-312 of SEQ ID NO: 4.

(full length human FGL1 amino acid sequence;
signal peptide is bolded)
SEQ ID NO: 4
MAKVFSFILVTTALTMGREISALEDCAQEQMRLRAQVRLLETRVKQQQ
VKIKQLLQENEVQFLDKGDENTVIDLGSKRQYADCSEIFNDGYKLSGF
YKIKPLQSPAEFSVYCDMSDGGGWTVIQRRSDGSENFNRGWKDYENGF
GNFVQKHGEYWLGNKNLHFLTTQEDYTLKIDLADFEKNSRYAQYKNFK
VGDEKNFYELNIGEYSGTAGDSLAGNFHPEVQWWASHQRMKFSTWDRD
HDNYEGNCAEEDQSGWWFNRCHSANLNGVYYSGPYTAKTDNGIVWYTW
HGWWYSLKSVVMKIRPNDFIPNVI

FGL1 is an immune suppressive molecule that inhibits antigen-specific T-cell activation by acting as a major ligand of LAG3. FGL1 is responsible for LAG3 T-cell inhibitory function. FGL1 binds LAG3 independently from MHC class II (MHC-II). FGL1 is secreted by and promotes the growth of hepatocytes.

### Galectin-9

In some embodiments, the ICL is galectin-9. In some embodiments, the ICL is human galectin-9. Sequences of galectin-9 molecules are known in the art. See, for example, GenBank ID NP_002299.2 and UniProtKB ID 000182. Galectin-9 is also known as Gal-9, ecalectin, LGALS9, or tumor antigen HOM-HD-21. In some embodiments, the human galectin-9 has the amino acid sequence of SEQ ID NO: 5 shown below. In some embodiments, the ICL comprises an amino acid sequence having at least about 85% (e.g., at least about any one of 86%, 87%, 88%, 89%, 90%, 91%, 92%, 93%, 94%, 95%, 96%, 97%, 98%, 99% or more) sequence identity to SEQ ID NO: 5.

(full length human galectin-9 amino acid
sequence)
SEQ ID NO: 5
MAFSGSQAPYLSPAVPFSGTIQGGLQDGLQITVNGTVLSSSGTRFAVN
FQTGFSGNDIAFHFNPRFEDGGYVVCNTRQNGSWGPEERKTHMPFQKG
MPFDLCFLVQSSDFKVMVNGILFVQYFHRVPFHRVDTISVNGSVQLSY
ISFQNPRTVPVQPAFSTVPFSQPVCFPPRPRGRRQKPPGVWPANPAPI
TQTVIHTVQSAPGQMFSTPAIPPMMYPHPAYPMPFITTILGGLYPSKS
ILLSGTVLPSAQRFHINLCSGNHIAFHLNPRFDENAVVRNTQIDNSWG
SEERSLPRKMPFVRGQSFSVWILCEAHCLKVAVDGQHLFEYYHRLRNL
PTINRLEVGGDIQLTHVQT

Galectin-9 is a ligand for the immune checkpoint molecule HAVCR2 (TIM3). Binding of galectin-9 to HAVCR2 induces T-helper type 1 lymphocyte (Th1) death. Galectin-9 is also a ligand for P4HB, which retains P4HB at the cell surface of Th2 T-helper cells, increasing disulfide reductase activity at the plasma membrane, altering the plasma membrane redox state and enhancing cell migration. Galectin-9 is a ligand for CD44. Its interaction with CD44 enhances binding of SMAD3 to the FOXP3 promoter, leading to up-regulation of FOXP3 expression and increased induced regulatory T (iTreg) cell stability and suppressive function (By similarity). Promotes ability of mesenchymal stromal cells to suppress T-cell proliferation. Galectin-9 is known to expand regulatory T-cells, induce cytotoxic T-cell apoptosis following virus infection, and inhibit NK cell function.

### CD47

In some embodiments, the ICL is CD47. In some embodiments, the ICL is human CD47. Sequences of PD-L1 molecules are known in the art. See, for example, GenBank ID CEJ95640.1 and UniProtKB ID 008722. CD47 is also known as leukocyte surface antigen CD47, antigenic surface determinant protein OA3, integrin-associated protein (IAP), or MER6. In some embodiments, the human CD47 has the amino acid sequence of SEQ ID NO: 6 shown below. In some embodiments, the ICL comprises an amino acid sequence having at least about 85% (e.g., at least about any one of 86%, 87%, 88%, 89%, 90%, 91%, 92%, 93%, 94%, 95%, 96%, 97%, 98%, 99%, or more) sequence identity to SEQ ID NO: 6. In some embodiments, the ICL is a functional fragment of a naturally occurring CD47. In some embodiments, the ICL is a full-length naturally occurring CD47. In some embodiment, the ICL comprises a signal peptide. In some embodiments, the ICL does not comprise a signal peptide. In some embodiments, the ICL comprises one or more extracellular domains of a naturally occurring CD47. In some embodiments, the ICL comprises one or more extracellular domains, transmembrane domains and cytoplasmic domains of a naturally occurring CD47. In some embodiments, the ICL comprises an amino acid sequence comprising amino acids 19-323 of SEQ ID NO: 6, or an amino acid sequence having at least about 85% (e.g., at least any one of 86%, 87%, 88%, 89%, 90%, 91%, 92%, 93%, 94%, 95%, 96%, 97%, 98%, 99%, or more) sequence identity to amino acids 19-323 of SEQ ID NO: 6.

(full length human CD47 amino acid sequence;
signal peptide is bolded)
SEQ ID NO: 6
MWPLVAALLLGSACCGSAQLLFNKTKSVEFTFCNDTVVIPCFVTNMEA
QNTTEVYVKWKFKGRDIYTFDGALNKSTVPTDFSSAKIEVSQLLKGDA
SLKMDKSDAVSHTGNYTCEVTELTREGETIIELKYRVVSWFSPNENIL
IVIFPIFAILLFWGQFGIKTLKYRSGGMDEKTIALLVAGLVITVIVIV
GAILFVPGEYSLKNATGLGLIVTSTGILILLHYYVFSTAIGLTSFVIA
ILVIQVIAYILAVVGLSLCIAACIPMHGPLLISGLSILALAQLLGLVY
MKFVASNQKTIQPPRKAVEEPLNAFKESKGMMNDE

CD47 is a transmembrane protein that interacts with integrins, some counter-receptor signal-regulatory protein (SIRP) family members, and secreted thrombospondin-1. CD47 has two established roles in the immune system. Its engagement of SIRPα on phagocytic cells and dendritic cells induces inhibitory signals that limit phagocytosis of CD47-expressing cells and antigen presentation. CD47-targeted therapeutics have been developed to overcome this immune checkpoint for cancer treatment. Additionally, engagement of CD47 by thrombospondin-1 inhibits T cell and NK cell activation. CD47 is, therefore, an immune checkpoint ligand that regulates both innate and adaptive immunity.

### B7H3

In some embodiments, the ICL is B7H3. In some embodiments, the ICL is human B7H3. Sequences of B7H3 molecules are known in the art. See, for example, GenBank ID NP_001316558.1 and UniProtKB ID 000182. B7H3 is also known as B7 homolog 3, CD276 antigen, 41g-B7-H3, and CD276. In some embodiments, the human B7H3 has the amino acid sequence of SEQ ID NO: 7 shown below. In some embodiments, the ICL comprises an amino acid sequence having at least about 85% (e.g., at least about any one of 86%, 87%, 88%, 89%, 90%, 91%, 92%, 93%, 94%, 95%, 96%, 97%, 98%, 99%, or more) sequence identity to SEQ ID NO: 7. In some embodiments, the ICL is a functional fragment of a naturally occurring B7H3. In some embodiments, the ICL is a full-length naturally occurring B7H3. In some embodiment, the ICL comprises a signal peptide. In some embodiments, the ICL does not comprise a signal peptide. In some embodiments, the ICL comprises one or more extracellular domains of a naturally occurring B7H3. In some embodiments, the ICL comprises one or more extracellular domains, transmembrane domains and cytoplasmic domains of a naturally occurring B71-13 In some embodiments, the ICL comprises an amino acid sequence comprising amino acids 29-534 of SEQ ID NO: 7, or an amino acid sequence having at least about 85% (e.g., at least any one of86%, 87%, 88%, 89%, 90%, 91%, 92%, 93%, 94%, 95%, 96%, 97%, 98%, 99%, or more) sequence identity to amino acids 29-534 of SEQ ID NO: 7.

(full length human B7H3 amino acid sequence;
signal peptide is bolded)
SEQ ID NO: 7
MLRRRGSPGMGVHVGAALGALWFCLTGALEVQVPEDPVVALVGTDATL
CCSFSPEPGFSLAQLNLIWQLTDTKQLVHSFAEGQDQGSAYANRTALF
PDLLAQGNASLRLQRVRVADEGSFTCFVSIRDFGSAAVSLQVAAPYSK
PSMTLEPNKDLRPGDTVTITCSSYQGYPEAEVFWQDGQGVPLTGNVTT
SQMANEQGLFDVHSILRVVLGANGTYSCLVRNPVLQQDAHSSVTITPQ
RSPTGAVEVQVPEDPVVALVGTDATLRCSFSPEPGFSLAQLNLIWQLT
DTKQLVHSFTEGRDQGSAYANRTALFPDLLAQGNASLRLQRVRVADEG
SFTCFVSIRDFGSAAVSLQVAAPYSKPSMTLEPNKDLRPGDTVTITCS
SYRGYPEAEVFWQDGQGVPLTGNVTTSQMANEQGLFDVHSVLRVVLGA
NGTYSCLVRNPVLQQDAHGSVTITGQPMTFPPEALWVTVGLSVCLIAL
LVALAFVCWRKIKQSCEEENAGAEDQDGEGEGSKTALQPLKHSDSKED
DGQEIA

B7-H3 (CD276) is a member of the B7 family of immunoregulatory molecules, many of which interact with known immune checkpoint proteins including CTLA-4, PD-1, and CD28. B7H3 has a co-inhibitory role on T-cells and contribute to immune evasion by tumor cells. B7-H3 is found to inhibit T-cell proliferation. B7-H3 has also been linked to decrease in secretion of IFN-7, TNF-α, and other cytokines, which allows for immune escape.

### B7H4

B7H4. In some embodiments, the ICL is human B7H4. Sequences of B7H4 molecules are known in the art. See, for example, GenBank ID NP_001240779.1.1 and UniProtKB ID Q7Z7D3. B7H4 is also known as V-set domain-containing T-cell activation inhibitor 1 (VTCN1), B7 homolog 4, B7h.5, B7S1, and T-cell costimulatory molecule B7x. In some embodiments, the human B7H4 has the amino acid sequence of SEQ ID NO: 8 shown below. In some embodiments, the ICL comprises an amino acid sequence having at least about 85% (e.g., at least about any one of 86%, 87%, 88%, 89%, 90%, 91%, 92%, 93%, 94%, 95%, 96%, 97, 98%, 99%, or more) sequence identity to SEQ ID NO: 8. In some embodiments, the ICL is a functional fragment of a naturally occurring B7H4 In some embodiments, the ICL is a full-length naturally occurring B7H4. In some embodiment, the ICL comprises a signal peptide. In some embodiments, the ICL does not comprise a signal peptide. In some embodiments, the ICL comprises one or more extracellular domains of a naturally occurring B7H4. In some embodiments, the ICL comprises one or more extracellular domains, transmembrane domains and cytoplasmic domains of a naturally occurring B7H4. In some embodiments, the ICL comprises an amino acid sequence comprising amino acids 25-282 of SEQ ID NO: 8, or an amino acid sequence having at least about 85% (e.g., at least any one of 86%, 87%, 88%, 89%, 90%6, 91%, 92%, 93%, 94%, 95%, 96%, 97%, 98%, 99%, or more) sequence identity to amino acids 25-282 of SEQ ID NO: 8.

(full length human B7H4 amino acid sequence,
signal peptide is bolded)
SEQ ID NO: 8
MASLGQILFWSIISIIIILAGAIALIIGFGISGRHSITVTTVASAGNI
GEDGILSCTFEPDIKLSDIVIQWLKEGVLGLVHEFKEGKDELSEQDEM
FRGRTAVFADQVIVGNASLRLKNVQLTDAGTYKCYIITSKGKGNANLE
YKTGAFSMPEVNVDYNASSETLRCEAPRWFPQPTVVWASQVDQGANFS
EVSNTSFELNSENVTMKVVSVLYNVTINNTYSCMIENDIAKATGDIKV
TESEIKRRSHLQLLNSKASLCVSSFFAISWALLPLSPYLMLK

B7H4 is a member of the B7 family of immunoregulatory proteins. B7H4 inhibits T cell proliferation and cytokine production through ligation of an unknown receptor expressed by activated T cells. Notably, B7H4 protein expression is observed in a high proportion of patients' tumors across a wide variety of malignancies. Preclinical studies of B7H4-specific chimeric antigen receptor (CAR) T cells, antibody-mediated blockade of B7H4, and anti-B7H4 drug conjugates have shown antitumor efficacy in mouse models.

### B. Engineered Receptor

The modified therapeutic cells described herein may express one or more engineered receptors. Exemplary engineered receptors include, but are not limited to, CAR, recombinant TCR, TAC receptor, and TFPs. In some embodiments, the engineered receptor comprises an extracellular domain that specifically binds to an antigen (e.g., a tumor antigen), a transmembrane domain, and an intracellular signaling domain. In some embodiments, the intracellular signaling domain comprises a primary intracellular signaling domain and/or a co-stimulatory domain. In some embodiments, the intracellular signaling domain comprises an intracellular signaling domain of a TCR co-receptor. In some embodiments, the engineered receptor is encoded by a heterologous nucleic acid operably linked to a promoter (such as a constitutive promoter or an inducible promoter). In some embodiments, the engineered receptor is introduced to the modified immune cell by inserting proteins into the cell membrane while passing cells through a microfluidic system, such as CELL SQUEEZE® (see, for example, U.S. Patent Application Publication No. 20140287509). The engineered receptor may enhance the function of the modified therapeutic cells, such as by targeting the modified therapeutic cells (e.g., modified immune cells), by transducing signals, and/or by enhancing cytotoxicity of the modified therapeutic cells (e.g., modified immune cells). In some embodiments, the modified therapeutic cell does not express an engineered receptor, such as CAR, TCR, TAC receptor, or TFP.

In some embodiments, the engineered receptor comprises one or more specific binding domains that target at least one tumor antigen, and one or more intracellular effector domains, such as one or more primary intracellular signaling domains and/or co-stimulatory domains.

In some embodiments, the engineered receptor is a chimeric antigen receptor (CAR). Many chimeric antigen receptors are known in the art and may be suitable for the modified therapeutic cells of the present invention. CARs can also be constructed with a specificity for any cell surface marker by utilizing antigen binding fragments or antibody variable domains of, for example, antibody molecules. Any method for producing a CAR may be used herein. See, for example, U.S. Pat. Nos. 6,410,319, 7,446,191, 7,514,537, 9,765,342B2, WO 2002/077029, WO2015/142675, US2010/065818, US 2010/025177, US 2007/059298, WO2017025038A1, and Berger C. et al., J. Clinical Investigation 118: 1 294-308 (2008), which are hereby incorporated by reference.

In some embodiments, a CAR comprises an extracellular domain comprising at least one targeting domain that specifically binds at least one tumor antigen, a transmembrane domain, and an intracellular signaling domain. In some embodiments, the intracellular signaling domain generates a signal that promotes an immune effector function of the CAR-containing cell, e.g., a CAR-T cell. “Immune effector function or immune effector response” refers to function or response, e.g., of an immune effector cell, that enhances or promotes an immune attack of a target cell. For example, an immune effector function or response may refer to a property of a T or NK cell that promotes killing or the inhibition of growth or proliferation, of a target cell. Examples of immune effector function, e.g., in a CAR-T cell, include cytolytic activity (such as antibody-dependent cellular toxicity, or ADCC) and helper activity (such as the secretion of cytokines). In some embodiments, the CAR has an intracellular signaling domain with an attenuated immune effector function. In some embodiments, the CAR has an intracellular signaling domain having no more than about any of 90%, 80%, 70%, 60%, 50%, 40%, 30%, 20%, 10% h or less of an immune effector function (such as cytolytic function against target cells) compared to a CAR having a full-length and wildtype CD3ζ and optionally one or more co-stimulatory domains. In some embodiments, the intracellular signaling domain generates a signal that promotes proliferation and/or survival of the CAR containing cell. In some embodiments, the CAR comprises one or more intracellular signaling domains selected from the signaling domains of CD28, CD137, CD3, CD27, CD40, ICOS, GITR, and OX40. The signaling domain of a naturally occurring molecule can comprise the entire intracellular (i.e., cytoplasmic) portion, or the entire native intracellular signaling domain, of the molecule, or a fragment or derivative thereof.

In some embodiments, the intracellular signaling domain of a CAR comprises a primary intracellular signaling domain. “Primary intracellular signaling domain” refers to cytoplasmic signaling sequence that acts in a stimulatory manner to induce immune effector functions. In some embodiments, the primary intracellular signaling domain contains a signaling motif known as Immunoreceptor Tyrosine-based Activation Motif, or ITAM. In some embodiments, the primary intracellular signaling domain comprises a functional signaling domain of a protein selected from the group consisting of CD3 zeta, CD3 gamma, CD3 delta, CD3 epsilon, common FcR gamma (FCER1G), FcR beta (Fc Epsilon Rib), CD79a, CD79b, Fcgamma RIIa, DAP10, and DAP12. In some embodiments, the primary intracellular signaling domain comprises a nonfunctional or attenuated signaling domain of a protein selected from the group consisting of CD3 zeta, CD3 gamma, CD3 delta, CD3 epsilon, common FcR gamma (FCER1G), FcR beta (Fc Epsilon Rib), CD79a, CD79b, Fcgamma RIIa, DAP10, and DAP12. The nonfunctional or attenuated signaling domain can be a mutant signaling domain having a point mutation, insertion or deletion that attenuates or abolishes one or more immune effector functions, such as cytolytic activity or helper activity, including antibody-dependent cellular toxicity (ADCC). In some embodiments, the CAR comprises a nonfunctional or attenuated CD3 zeta (i.e. CD3ζ or CD3z) signaling domain. In some embodiments, the intracellular signaling domain does not comprise a primary intracellular signaling domain. An attenuated primary intracellular signaling domain may induce no more than about any of 90%, 80%, 70%, 60%, 50%, 40%, 30%, 20%, 10% or less of an immune effector function (such as cytolytic function against target cells) compared to CARs having the same construct, but with the wildtype primary intracellular signaling domain.

In some embodiments, the intracellular signaling domain of a CAR comprises one or more (such as any of 1, 2, 3, or more) co-stimulatory domains. “Co-stimulatory domain” can be the intracellular portion of a co-stimulatory molecule. The term “co-stimulatory molecule” refers to a cognate binding partner on an immune cell (such as T cell) that specifically binds with a co-stimulatory ligand, thereby mediating a co-stimulatory response by the immune cell, such as, but not limited to, proliferation and survival. Co-stimulatory molecules are cell surface molecules other than antigen receptors or their ligands that contribute to an efficient immune response. A co-stimulatory molecule can be represented in the following protein families: TNF receptor proteins, Immunoglobulin-like proteins, cytokine receptors, integrins, signaling lymphocytic activation molecules (SLAM proteins), and activating NK cell receptors. Co-stimulatory molecules include, but are not limited to an MHC class I molecule, BTLA and a Toll ligand receptor, as well as OX40, CD27, CD28, CDS, ICAM-1, LFA-1 (CD11a/CD18), ICOS (CD278), and 4-1BB (CD137). Further examples of such co-stimulatory molecules include CD5, ICAM-1, GITR, BAFFR, HVEM (LIGHTR), SLAMF7, NKp80 (KLRF1), NKp44, NKp30, NKp46, CD160, CD19, CD4, CD8alpha, CD8beta, IL-2R beta, IL-2R gamma, IL-7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CDIOO (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and a ligand that specifically binds with CD83.

In some embodiments, the CAR comprises a single co-stimulatory domain. In some embodiments, the CAR comprises two or more co-stimulatory domains. In some embodiments, the intracellular signaling domain comprises a functional primary intracellular signaling domain and one or more co-stimulatory domains. In some embodiments, the CAR does not comprise a functional primary intracellular signaling domain (such as CD3ζ). In some embodiments, the CAR comprises an intracellular signaling domain consisting of or consisting essentially of one or more co-stimulatory domains. In some embodiments, the CAR comprises an intracellular signaling domain consisting of or consisting essentially of a nonfunctional or attenuated primary intracellular signaling domain (such as a mutant CD3ζ) and one or more co-stimulatory domains. Upon binding of the targeting domain to tumor antigen, the co-stimulatory domains of the CAR may transduce signals for enhanced proliferation, survival and differentiation of the engineered immune cells having the CAR (such as T cells), and inhibit activation induced cell death. In some embodiments, the one or more co-stimulatory signaling domains are derived from one or more molecules selected from the group consisting of CD27, CD28, 4-1BB (i.e., CD137), OX40, CD30, CD40, CD3, lymphocyte function-associated antigen-1 (LFA-1), CD2, CD7, LIGHT, NKG2C, B7-H3 and ligands that specially bind to CD83.

In some embodiments, the intracellular signaling domain of the CAR comprises a co-stimulatory signaling domain derived from CD28. In some embodiments, the intracellular signaling domain comprises a cytoplasmic signaling domain of CD3ζ and a co-stimulatory signaling domain of CD28. In some embodiments, the intracellular signaling domain in the chimeric receptor of the present application comprises a co-stimulatory signaling domain derived from 4-1BB (i.e., CD137). In some embodiments, the intracellular signaling domain comprises a cytoplasmic signaling domain of CD3ζ and a co-stimulatory signaling domain of 4-1BB.

In some embodiments, the intracellular signaling domain of the CAR comprises a co-stimulatory signaling domain of CD28 and a co-stimulatory signaling domain of 4-1BB. In some embodiments, the intracellular signaling domain comprises a cytoplasmic signaling domain of CD3ζ, a co-stimulatory signaling domain of CD28, and a co-stimulatory signaling domain of 4-1BB. In some embodiments, the intracellular signaling domain comprises a polypeptide comprising from the N-terminus to the C-terminus: a co-stimulatory signaling domain of CD28, a co-stimulatory signaling domain of 4-1BB, and a cytoplasmic signaling domain of CD3ζ.

In some embodiments, the CAR comprises a polypeptide comprising from the N-terminus to the C-terminus: a CD8 leader, an extracellular binding domain, a CD8 hinge, a CD8 transmembrane, a 4-1BB intracellular co-stimulatory domain, and a CD3ζ intracellular signaling domain.

In some embodiments, the CAR is a chimeric signaling domain (“CMSD”)-containing chimeric antigen receptor, wherein the CMSD comprises ITAMs (also referred to herein as “CMSD ITAMs”) and optional linkers (also referred to herein as “CMSD linkers”) arranged in a configuration that is different from any of the naturally occurring ITAM-containing parent molecules. For example, in some embodiments, the CMSD comprises two or more ITAMs directly linked to each other. In some embodiments, the CMSD comprises ITAMs connected by one or more “heterologous linkers”, namely, linker sequences which are either not derived from an ITAM-containing parent molecule (e.g., G/S linkers), or derive from an ITAM-containing parent molecule that is different from the ITAM-containing parent molecule from which one or more of the CMSD ITAMs are derived from. In some embodiments, the CMSD comprises two or more (such as 2, 3, 4, or more) identical ITAMs. In some embodiments, at least two of the CMSD ITAMs are different from each other. In some embodiments, at least one of the CMSD ITAMs is not derived from CD3ζ. In some embodiments, at least one of the CMSD ITAMs is not ITAM1 or ITAM2 of CD3ζ. In some embodiments, the CMSD does not comprise CD3ζ ITAM1 and/or CD3ζ ITAM2. In some embodiments, at least one of the CMSD ITAMs is CD3ζ ITAM3. In some embodiments, the CMSD does not comprise any ITAMs from CD3ζ. In some embodiments, at least two of the CMSD ITAMs are derived from the same ITAM-containing parent molecule. In some embodiments, the CMSD comprises two or more (such as 2, 3, 4, or more) ITAMs, wherein at least two of the CMSD ITAMs are each derived from a different ITAM-containing parent molecule. In some embodiments, at least one of the CMSD ITAMs is derived from an ITAM-containing parent molecule selected from the group consisting of: CD3ε, CD3δ, CD3γ, Igα (CD79a), Igβ (CD79b), FcεRIβ, FcεRIγ, DAP12, CNAIP/NFAM1, STAM-1, STAM-2, and Moesin.

In some embodiments, the CAR comprises a polypeptide comprising from the N-terminus to the C-terminus: a CD8 leader, an extracellular binding domain, a CD8 hinge, a CD8 transmembrane, a 4-1BB intracellular co-stimulatory domain, and one or more ITAM sequences.

In some embodiments, the targeting domain of the CAR is an antibody or an antibody fragment, such as an scFv, a Fv, a Fab, a (Fab′)₂, a single domain antibody (sdAb), or a V_{H}H domain. In some embodiments, the targeting domain of the CAR is a ligand or an extracellular portion of a receptor that specifically binds to a tumor antigen. In some embodiments, the one or more targeting domains of the CAR specifically bind to a single tumor antigen. In some embodiments, the CAR is a bispecific or multispecific CAR with targeting domains that bind two or more tumor antigens. In some embodiments, the tumor antigen is selected from the group consisting of CD19, BCMA, NY-ESO-1, VEGFR2, MAGE-A3, CD20, CD22, CD33, CD38, CEA, EGFR (such as EGFRvIII), GD2, HER2, IGF1R, mesothelin, PSMA, ROR1, WT1 and other tumor antigens with clinical significance, and combinations thereof.

In some embodiments, the CAR is an anti-CD19 CAR. In some embodiments, the CAR comprises an anti-CD19 scFv. In some embodiments, the anti-CD19 scFv is derived from an anti-CD19 antibody such as FMC63. In some embodiments, the CAR is an anti-CD19 CAR comprising the amino acid sequence of SEQ ID NO: 9.

(CTL-019 CAR; CD8α SP-CD19
scFv-CD8α hinge-CD8α TM-4-1BB-CD3ζ
amino acid sequence; CD8α SP is italicized,
CD8α hinge is squared, CD8α TM is italicized,
4-1BB cytoplasmic is underlined, CD3ζ is bolded)
SEQ ID NO: 9
MALPVTALLLPLALLLHAARPDIQMTQTTSSLSASLGDRVTI
SCRASQDISKYLNWYQQKPDGTVKLLIYHTSRLHSGVPS
RFSGSGSGTDYSLTISNLEQEDIATYFCQQGNTLPYTFGG
GTKLEITGGGGSGGGGSGGGGSEVKLQESGPGLVAPSQ
SLSVTCTVSGVSLPDYGVSWIRQPPRKGLEWLGVIW
GSETTYYNSALKSRLTIIKDNSKSQVFLKMNSLQTDD
TAIYYCAKHYYYGGSYAMDYWGQGTSVTVSS

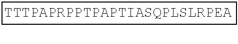

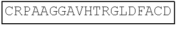

IYIWAPLAGTCGVLLLSLVITLYCKRGRKKLLYIFKQPFM
RPVQTTQEEDGCSCRFPEEEEGGCELRVKFSRSADAP
AYKQGQNQLYNELNLGRREEYDVLDKRRGRDPE
MGGKPRRKNPQEGLYNELQKDKMAEAYSEIGMKGERR
RGKGHDGLYQGLSTATKDTYDALHMQALPPR

In some embodiments, the CAR is an anti-BCMA CAR. A wide variety of antigen binding domain sequences can be used as the targeting domains of the CAR. See, e.g., WO2017/025038, which is incorporated herein in its entirety. In some embodiments, the CAR comprises an anti-BCMA scFv. In some embodiments, the CAR comprises an anti-BCMA sdAb, such as V_{H}H. In some embodiments, the CAR is an anti-BCMA CAR comprising the amino acid sequence of SEQ ID NO: 10.

(BSF17 CAR; CD8α SP-BCMA V_{H}H-CD8α
hinge-CD8α TM-4-1BB-CD3ζ
amino acid sequence; CD8α SP is italicized,
CD8α hinge is squared, CD8α TM is italicized,
4-1BB cytoplasmic is underlined, CD3ζ is bolded)
SEQ ID NO: 10
MALPVTALLLPLALLLHAARPAVQLVESGGGLVQAGD
SLRLTCTASGRAFSTYFMAWFRQAPGKEREFVAGIAW
SGGSTAYADSVKGRFTISRDNAKNTVYLQMNSLKSEDTAV
YYCASRGIEVEEFGAWGQGTQVTVSSGGGGSQVQLEESGG
GSVQAGGSLRLSCAYTYSTYSNYYMGWFREAPGKARTSV
AIISSDTTITYKDAVKGRFTISKDNAKNTLYLQM
NSLKPEDSAMYRCAAWTSDWSVAYWGQGTQVTVSSTS

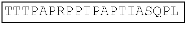

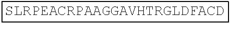

IYIWAPLAGTCGVLLLSLVITLYCKRGRKKLLYIF
KQPFMRPVQTTQEEDGCSCRFPEEEEGGCEL
RVKFSRSADAPAYQQGQNQLYNELNLGRREEYDVLDK
RRGRDPEMGGKPRRKNPQEGLYNELQKDKMAEAYSEIG
MKGERRRGKGHDGLYQGLSTATKDTYDALHMQALPPR

In some embodiments, the transmembrane domain of the CAR comprises a transmembrane domain chosen from the transmembrane domain of an alpha, beta or zeta chain of a T-cell receptor, CD28, CD3 epsilon, CD45, CD4, CD5, CD8, CD9, CD16, CD22, CD33, CD37, CD64, CD80, CD86, CD134, CD137, CD154, KIRDS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), CD160, CD19, IL-2R beta, IL-2R gamma, IL-7Ra, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRT AM, Ly9 (CD229), CD160 (BY55), PSGL1, CDIOO (SEMA4D), SLAMF6 (NTB-A, Ly108), SLAM (SLAMF1, CD150, IPO-3), BLAME (SLAMF8), SELPLG (CD162), LTBR, PAG/Cbp, NKp44, NKp30, NKp46, NKG2D, and/or NKG2C. In some embodiments, the transmembrane domain of the CAR is a CD4, CD3, CD8α, or CD28 transmembrane domain. In some embodiments, the transmembrane domain of the CAR comprises a transmembrane domain of CD8α.

In some embodiments, the extracellular domain is connected to the transmembrane domain by a hinge region. In one embodiment, the hinge region comprises the hinge region of CD8α.

In some embodiments, the CAR comprises a signal peptide, such as a CD8αSP.

Any CAR known in the art or developed by the inventors, including the CARs described in PCT/CN2017/096938 and PCT/CN2016/094408 (the contents of which are incorporated herein by reference in their entireties), may be used in the methods described herein. Exemplary structures of CARs are shown in FIGS. 15A-15D of PCT/CN2017/096938.

In some embodiments, the engineered receptor is a recombinant T-cell receptor. In some embodiments, the recombinant TCR is specific for a tumor antigen. In some embodiments, the tumor antigen is selected from the group consisting of CD19, BCMA, NY-ESO-1, VEGFR2, MAGE-A3, VEGFR2, MAGE-A3, CD20, CD22, CD33, CD38, CEA, EGFR (such as EGFRvIII), GD2, HER2, IGF1R, mesothelin, PSMA, ROR1, WT1 and other tumor antigens with clinical significance. In some embodiments, the tumor antigen is derived from an intracellular protein of tumor cells. Many TCRs specific for tumor antigens (including tumor-associated antigens) have been described, including, for example, NY-ESO-1 cancer-testis antigen, the p53 tumor suppressor antigens, TCRs for tumor antigens in melanoma (e.g., MARTI, gp 100), leukemia (e.g., WT1, minor histocompatibility antigens), and breast cancer (HER2, NY-BR1, for example). Any of the TCRs known in the art may be used in the present application. In some embodiments, the TCR has an enhanced affinity to the tumor antigen. Exemplary TCRs and methods for introducing the TCRs to immune cells have been described, for example, in U.S. Pat. No. 5,830,755, and Kessels et al. Immunotherapy through TCR gene transfer. Nat. Immunol. 2, 957-961 (2001). In some embodiments, the modified therapeutic cell is a TCR-T cell.

The TCR receptor complex is an octomeric complex formed by variable TCR receptor α and β chains (γ and δ chains on case of γδ T cells) with three dimeric signaling modules CD3δ/ε, CD3γ/ε and CD247 (T-cell surface glycoprotein CD3 zeta chain) ζ/ζ or ζ/η. Ionizable residues in the transmembrane domain of each subunit form a polar network of interactions that hold the complex together. TCR complex has the function of activating signaling cascades in T cells.

In some embodiments, the engineered receptor is an engineered TCR comprising one or more T-cell receptor (TCR) fusion proteins (TFPs). Exemplary TFPs have been described, for example, in US20170166622A1, which is incorporated herein by reference. In some embodiments, the TFP comprises an extracellular domain of a TCR subunit that comprises an extracellular domain or portion thereof of a protein selected from the group consisting of a TCR alpha chain, a TCR beta chain, a CD3 epsilon TCR subunit, a CD3 gamma TCR subunit, a CD3 delta TCR subunit, functional fragments thereof, and amino acid sequences thereof having at least one but not more than 20 modifications. In some embodiments, the TFP comprises a transmembrane domain that comprises a transmembrane domain of a protein selected from the group consisting of a TCR alpha chain, a TCR beta chain, a CD3 epsilon TCR subunit, a CD3 gamma TCR subunit, a CD3 delta TCR subunit, functional fragments thereof, and amino acid sequences thereof having at least one but not more than 20 modifications. In some embodiments, the TFP comprises a transmembrane domain that comprises a transmembrane domain of a protein selected from the group consisting of a TCR alpha chain, a TCR beta chain, a TCR zeta chain, a CD3 epsilon TCR subunit, a CD3 gamma TCR subunit, a CD3 delta TCR subunit, CD45, CD4, CD5, CD8, CD9, CD16, CD22, CD33, CD28, CD37, CD64, CD80, CD86, CD134, CD137, CD154, functional fragments thereof, and amino acid sequences thereof having at least one but not more than 20 modifications.

In some embodiments, the TFP comprising a TCR subunit comprising at least a portion of a TCR extracellular domain, and a TCR intracellular domain comprising a stimulatory domain from an intracellular signaling domain of CD3 epsilon; and an antigen binding domain, wherein the TCR subunit and the antigen binding domain are operatively linked, and wherein the TFP incorporates into a TCR when expressed in a T cell.

In some embodiments, the TFP comprises a TCR subunit comprising at least a portion of a TCR extracellular domain, and a TCR intracellular domain comprising a stimulatory domain from an intracellular signaling domain of CD3 gamma; and an antigen binding domain wherein the TCR subunit and the antigen binding domain are operatively linked, and wherein the TFP incorporates into a TCR when expressed in a T cell.

In some embodiments, the TFP comprises a TCR subunit comprising at least a portion of a TCR extracellular domain, and a TCR intracellular domain comprising a stimulatory domain from an intracellular signaling domain of CD3 delta; and an antigen binding domain, wherein the TCR subunit and the antigen binding domain are operatively linked, and wherein the TFP incorporates into a TCR when expressed in a T cell.

In some embodiments, the TFP comprises a TCR subunit comprising at least a portion of a TCR extracellular domain, and a TCR intracellular domain comprising a stimulatory domain from an intracellular signaling domain of TCR alpha; and an antigen binding domain wherein the TCR subunit and the antigen binding domain are operatively linked, and wherein the TFP incorporates into a TCR when expressed in a T cell.

In some embodiments, the TFP comprises a TCR subunit comprising at least a portion of a TCR extracellular domain, and a TCR intracellular domain comprising a stimulatory domain from an intracellular signaling domain of TCR beta; and an antigen binding domain wherein the TCR subunit and the antigen binding domain are operatively linked, and wherein the TFP incorporates into a TCR when expressed in a T cell.

In some embodiments, the engineered receptor is a T-cell antigen coupler (TAC) receptor. Exemplary TAC receptors have been described, for example, in US20160368964A1, which is incorporated herein by reference. In some embodiments, the TAC comprises a targeting domain, a TCR-binding domain that specifically binds a protein associated with the TCR complex, and a T-cell receptor signaling domain. In some embodiments, the targeting domain is an antibody fragment, such as scFv or V_{H}H, which specifically binds to a tumor antigen. In some embodiments, the targeting domain is a designed Ankyrin repeat (DARPin) polypeptide. In some embodiments, the tumor antigen is selected from the group consisting of CD19, BCMA, NY-ESO-1, VEGFR2, MAGE-A3, VEGFR2, MAGE-A3, CD20, CD22, CD33, CD38, CEA, EGFR (such as EGFRvIII), GD2, HER2, IGF1R, mesothelin, PSMA, ROR1, WT1 and other tumor antigens with clinical significance. In some embodiments, the protein associated with the TCR complex is CD3, such as CD3ε. In some embodiments, the TCR-binding domain is a single chain antibody, such as scFv, or a V_{H}H. In some embodiments, the TCR-binding domain is derived from UCHT1. In some embodiments, the TAC receptor comprises a cytosolic domain and a transmembrane domain. In some embodiments, the T-cell receptor signaling domain comprises a cytosolic domain derived from a TCR co-receptor. Exemplary TCR co-receptors include, but are not limited to, CD4, CD8, CD28, CD45, CD4, CD5, CD9, CD16, CD22, CD33, CD37, CD64, CD80, CD86, CD134, CD137 and CD154. In some embodiments, the TAC receptor comprises a transmembrane domain and a cytosolic domain derived from CD4. In some embodiments, the TAC receptor comprises a transmembrane domain and a cytosolic domain derived from CD8 (such as CD8α).

T cell co-receptors are expressed as membrane proteins on T cells. They can provide stabilization of the TCR: peptide: MHC complex and facilitate signal transduction. The two subtypes of T cell co-receptor, CD4 and CD8, display strong specificity for particular MHC classes. The CD4 co-receptor can only stabilize TCR: MHC II complexes while the CD8 co-receptor can only stabilize the TCR: MHC I complex. The differential expression of CD4 and CD8 on different T cell types results in distinct T cell functional subpopulations. CD8⁺ T cells are cytotoxic T cells.

CD4 is a glycoprotein expressed on the surface of immune cells such as T helper cells, monocytes, macrophages, and dendritic cells. CD4 has four immunoglobulin domains (D₁ to D₄) exposed on the extracellular cell surface. CD4 contains a special sequence of amino acids on its short cytoplasmic/intracellular tail, which allow CD4 tail to recruit and interact with the tyrosine kinase Lck. When the TCR complex and CD4 each bind to distinct regions of the MHC II molecule, the close proximity between the TCR complex and CD4 allows Lck bound to the cytoplasmic tail of CD4 to tyrosine-phosphorylate the Immunoreceptor Tyrosine Activation Motifs (ITAM) on the cytoplasmic domains of CD3, thus amplifying TCR generated signal.

CD8 is a glycoprotein of either a homodimer composed of two a chains (less common), or a heterodimer composed of one a and one β chain (more common), each comprising an immunoglobulin variable (IgV)-like extracellular domain connected to the membrane by a thin stalk, and an intracellular tail. CD8 is predominantly expressed on the surface of cytotoxic T cells, but can also be found on natural killer cells, cortical thymocytes, and dendritic cells. The CD8 cytoplasmic tail interacts with Lck, which phosphorylates the cytoplasmic CD3 and ζ-chains of the TCR complex once TCR binds its specific antigen. Tyrosine-phosphorylation on the cytoplasmic CD3 and ζ-chains initiates a cascade of phosphorylation, eventually leading to gene transcription.

In some embodiments, the modified therapeutic cell expresses more than one engineered receptor, such as any combination of CAR, recombinant TCRs, TAC receptors and TFPs.

In some embodiments, the engineered receptor (such as CAR, TCR, TAC or TFP) expressed by the modified therapeutic cell targets one or more tumor antigens. Tumor antigens are proteins that are produced by tumor cells that can elicit an immune response, particularly T-cell mediated immune responses. The selection of the targeted antigen of the invention will depend on the particular type of cancer to be treated. Exemplary tumor antigens include, for example, a glioma-associated antigen, carcinoembryonic antigen (CEA), β-human chorionic gonadotropin, alphafetoprotein (AFP), lectin-reactive AFP, thyroglobulin, RAGE-1, MN-CAIX, human telomerase reverse transcriptase, RU1, RU2 (AS), intestinal carboxyl esterase, mut hsp70-2, M-CSF, prostase, prostate-specific antigen (PSA), PAP, NY-ESO-1, LAGE-1a, p53, prostein, PSMA, HER2/neu, survivin and telomerase, prostate-carcinoma tumor antigen-1 (PCTA-1), MAGE, ELF2M, neutrophil elastase, ephrinB2, CD22, insulin growth factor (IGF)-I, IGF-II, IGF-I receptor and mesothelin.

In some embodiments, the tumor antigen comprises one or more antigenic cancer epitopes associated with a malignant tumor. Malignant tumors express a number of proteins that can serve as target antigens for an immune attack. These molecules include but are not limited to tissue-specific antigens such as MART-1, tyrosinase and gp100 in melanoma and prostatic acid phosphatase (PAP) and prostate-specific antigen (PSA) in prostate cancer. Other target molecules belong to the group of transformation-related molecules such as the oncogene HER2/Neu/ErbB-2. Yet another group of target antigens are onco-fetal antigens such as carcinoembryonic antigen (CEA). In B-cell lymphoma, the tumor-specific idiotype immunoglobulin constitutes a truly tumor-specific immunoglobulin antigen that is unique to the individual tumor. B cell differentiation antigens such as CD19, CD20 and CD37 are other candidates for target antigens in B-cell lymphoma.

In some embodiments, the tumor antigen is a tumor-specific antigen (TSA) or a tumor-associated antigen (TAA). A TSA is unique to tumor cells and does not occur on other cells in the body. A TAA is not unique to a tumor cell, and instead is also expressed on a normal cell under conditions that fail to induce a state of immunologic tolerance to the antigen. The expression of the antigen on the tumor may occur under conditions that enable the immune system to respond to the antigen. TAAs may be antigens that are expressed on normal cells during fetal development, when the immune system is immature, and unable to respond or they may be antigens that are normally present at extremely low levels on normal cells, but which are expressed at much higher levels on tumor cells.

Non-limiting examples of TSA or TAA antigens include the following: Differentiation antigens such as MART-1/MelanA (MART-), gp 100 (Pmel 17), tyrosinase, TRP-1, TRP-2 and tumor-specific multilineage antigens such as MAGE-1, MAGE-3, BAGE, GAGE-1, GAGE-2, p15; overexpressed embryonic antigens such as CEA; overexpressed oncogenes and mutated tumor-suppressor genes such as p53, Ras, HER2/neu; unique tumor antigens resulting from chromosomal translocations; such as BCR-ABL, E2A-PRL, H4-RET, IGH-IGK, MYL-RAR; and viral antigens, such as the Epstein Barr virus antigens EBVA and the human papillomavirus (HPV) antigens E6 and E7. Other large, protein-based antigens include TSP-180, MAGE-4, MAGE-5, MAGE-6, RAGE, NY-ESO, p185erbB2, p180erbB-3, c-met, nm-23H1, PSA, TAG-72, CA 19-9, CA 72-4, CAM 17.1, NuMa, K-ras, beta-Catenin, CDK4, Mum-1, p 15, p 16, 43-9F, 5T4, 791Tgp72, alpha-fetoprotein, beta-HCG, BCA225, BTAA, CA 125, CA 15-3\CA 27.29\BCAA, CA 195, CA 242, CA-50, CAM43, CD68\P1, CO-029, FGF-5, G250, Ga733\EpCAM, HTgp-175, M344, MA-50, MG7-Ag, MOV18, NB/70K, NY-CO-1, RCAS 1, SDCCAG16, TA-90\Mac-2 binding protein\cyclophilin C-associated protein, TAAL6, TAG72, TLP, and TPS.

### C. Nucleic Acids

The modified therapeutic cells described herein comprise one or more nucleic acids comprising heterologous nucleic acid sequence(s) encoding any one of the immune checkpoint ligands (ICLs) and/or engineered receptors described herein. In some embodiments, the nucleic acid is a DNA. In some embodiments, the nucleic acid is a RNA. In some embodiments, the nucleic acid is linear. In some embodiments, the nucleic acid is circular.

The heterologous nucleic acid sequence(s) may be operably linked to one or more regulatory sequences. Exemplary regulatory sequences that control the transcription and/or translation of a coding sequence are known in the art and may include, but not limited to, a promoter, additional elements for proper initiation, regulation and/or termination of transcription (e.g. polyA transcription termination sequences), mRNA transport (e.g. nuclear localization signal sequences), processing (e.g. splicing signals), stability (e.g. introns and non-coding 5′ and 3′ sequences), translation (e.g. an initiator Met, tripartite leader sequences, IRES ribosome binding sites, signal peptides, etc.), and insertion site for introducing an insert into the viral vector. In some embodiments, the regulatory sequence is a promoter, a transcriptional enhancer and/or a sequence that allows for proper expression of the ICL and/or the engineered receptor.

The term “regulatory sequence” or “control sequence” refers to a DNA sequence that affects the expression of a coding sequence to which it is operably linked. The nature of such regulatory sequences differs depending upon the host organism. In prokaryotes, regulatory sequences generally include promoters, ribosomal binding sites, and terminators. In eukaryotes, regulatory sequences include promoters, terminators and, in some instances, enhancers, transactivators or transcription factors.

The term “operably linked” refers to a juxtaposition wherein the components so described are in a relationship permitting them to function in their intended manner. A regulatory sequence “operably linked” to a coding sequence is ligated in such a way that expression of the coding sequence is achieved under conditions compatible with the regulatory sequences.

As used herein, a “promoter” or a “promoter region” refers to a segment of DNA or RNA that controls transcription of the DNA or RNA to which it is operatively linked. The promoter region includes specific sequences that are involved in RNA polymerase recognition, binding and transcription initiation. In addition, the promoter includes sequences that modulate recognition, binding and transcription initiation activity of RNA polymerase (i.e., binding of one or more transcription factors). These sequences can be cis acting or can be responsive to trans acting factors. Promoters, depending upon the nature of the regulation, can be constitutive or regulated. Regulated promoters can be inducible or environmentally responsive (e.g. respond to cues such as pH, anaerobic conditions, osmoticum, temperature, light, or cell density). Many such promoter sequences are known in the art. See, for example, U.S. Pat. Nos. 4,980,285; 5,631,150; 5,707,928; 5,759,828; 5,888,783; 5,919,670, and, Sambrook, et al., Molecular Cloning: A Laboratory Manual, 2nd Ed., Cold Spring Harbor Press (1989).

In some embodiments, the promoter is an endogenous promoter. For example, a nucleic acid sequencing encoding the engineered receptor may be knocked-in to the genome of a modified immune cell downstream of an endogenous promoter using any methods known in the art, such as CRISPR/Cas9 method. In some embodiments, the endogenous promoter is a promoter for an abundant protein, such as beta-actin. In some embodiments, the endogenous promoter is an inducible promoter, for example, inducible by an endogenous activation signal of the modified therapeutic cell (e.g., modified immune cell). In some embodiments, wherein the modified therapeutic cell is a T cell, the promoter is a T cell activation-dependent promoter (such as an IL-2 promoter, an NFAT promoter, or an NFκB promoter). In some embodiments, the promoter is a heterologous promoter.

Varieties of promoters have been explored for gene expression in mammalian cells, and any of the promoters known in the art may be used in the present invention. Promoters may be roughly categorized as constitutive promoters or regulated promoters, such as inducible promoters. In some embodiments, the heterologous nucleic acid sequence encoding the engineered receptor is operably linked to a constitutive promoter. In some embodiments, the heterologous nucleic acid sequence encoding the engineered receptor is operably linked to an inducible promoter.

Constitutive promoters allow heterologous genes (also referred to as transgenes) to be expressed constitutively in the host cells. Exemplary constitutive promoters contemplated herein include, but are not limited to, Cytomegalovirus (CMV) promoters, human elongation factors-1alpha (hEF1α), ubiquitin C promoter (UbiC), phosphoglycerokinase promoter (PGK), simian virus 40 early promoter (SV40), and chicken β-Actin promoter coupled with CMV early enhancer (CAGG). The efficiencies of such constitutive promoters on driving transgene expression have been widely compared in a huge number of studies. In some embodiments, the promoter is a hEF1α promoter.

In some embodiments, the promoter is an inducible promoter. Inducible promoters belong to the category of regulated promoters. The inducible promoter can be induced by one or more conditions, such as a physical condition, microenvironment of the modified therapeutic cell (e.g., modified immune cell), or the physiological state of the modified therapeutic cell (e.g., modified immune cell), an inducer (i.e., an inducing agent), or a combination thereof. In some embodiments, the inducing condition does not induce the expression of endogenous genes in the modified therapeutic cell (e.g., modified immune cell), and/or in the subject that receives the pharmaceutical composition. In some embodiments, the inducing condition is selected from the group consisting of: inducer, irradiation (such as ionizing radiation, light), temperature (such as heat), redox state, tumor environment, and the activation state of the modified therapeutic cell (e.g., modified immune cell).

In some embodiments, the promoter is inducible by an inducer. In some embodiments, the inducer is a small molecule, such as a chemical compound. In some embodiments, the small molecule is selected from the group consisting of doxycycline, tetracycline, alcohol, metal, or steroids. Chemically-induced promoters have been most widely explored. Such promoters includes promoters whose transcriptional activity is regulated by the presence or absence of a small molecule chemical, such as doxycycline, tetracycline, alcohol, steroids, metal and other compounds. Doxycycline-inducible system with reverse tetracycline-controlled transactivator (rtTA) and tetracycline-responsive element promoter (TRE) is the most established system at present. WO9429442 describes the tight control of gene expression in eukaryotic cells by tetracycline responsive promoters. WO9601313 discloses tetracycline-regulated transcriptional modulators. Additionally, Tet technology, such as the Tet-on system, has described, for example, on the website of TetSystems.com. Any of the known chemically regulated promoters may be used to drive expression of the therapeutic protein in the present application.

In some embodiments, the inducer is a polypeptide, such as a growth factor, a hormone, or a ligand to a cell surface receptor, for example, a polypeptide that specifically binds a tumor antigen. Many polypeptide inducers are also known in the art, and they may be suitable for use in the present invention. For example, ecdysone receptor-based gene switches, progesterone receptor-based gene switches, and estrogen receptor based gene switches belong to gene switches employing steroid receptor derived transactivators (WO9637609 and WO9738117 etc.).

In some embodiments, the inducer comprises both a small molecule component and one or more polypeptides. For example, inducible promoters that dependent on dimerization of polypeptides are known in the art, and may be suitable for use in the present invention. The first small molecule CID system, developed in 1993, used FK1012, a derivative of the drug FK506, to induce homo-dimerization of FKBP. By employing similar strategies, Wu et al successfully make the CAR-T cells titratable through an ON-switch manner by using Rapalog/FKPB-FRB® and Gibberelline/GID1-GAI dimerization dependent gene switch (C.-Y. Wu et al., Science 350, aab4077 (2015)). Other dimerization dependent switch systems include Coumermycin/GyrB-GyrB (Nature 383 (6596): 178-81), and HaXS/Snap-tag-HaloTag (Chemistry and Biology 20 (4); 549-57).

In some embodiments, the promoter is a light-inducible promoter, and the inducing condition is light. Light inducible promoters for regulating gene expression in mammalian cells are also well-known in the art (see, for example, Science 332, 1565-1568 (2011), Nat. Methods 9, 266-269 (2012); Nature 500: 472-476 (2013); Nature Neuroscience 18:1202-1212 (2015)). Such gene regulation systems can be roughly divided into two categories based on their regulations of (1) DNA binding or (2) recruitment of a transcriptional activation domain to a DNA bound protein. For instance, synthetic mammalian blue light controlled transcription system based on melanopsin, which, in response to blue light (480 nm), triggers an intracellular calcium increase that result in calcineurin-mediated mobilization of NFAT, were developed and tested in mammalian cells. More recently, Motta-Mena et al described a new inducible gene expression system developed from naturally occurring EL222 transcription factor that confers high-level, blue light-sensitive control of transcriptional initiation in human cell lines and zebrafish embryos (Nat. Chem. Biol. 10(3):196-202 (2014)). Additionally, the red light induced interaction of photoreceptor phytochrome B (PhyB) and phytochrome-interacting factor 6 (PIF6) of Arabidopsis thaliana was exploited for a red light triggered gene expression regulation. Furthermore, ultraviolet B (UVB)-inducible gene expression system were also developed and proven to be efficient in target gene transcription in mammalian cells (Chapter 25 of Gene and Cell Therapy: Therapeutic Mechanisms and Strategies, Fourth Edition CRC Press, Jan. 20, 2015). Any of the light-inducible promoters described herein may be used to drive expression of the therapeutic protein in the present invention.

In some embodiments, the promoter is a light-inducible promoter that is induced by a combination of a light-inducible molecule, and light. For example, a light-cleavable photocaged group on a chemical inducer keeps the inducer inactive, unless the photocaged group is removed through irradiation or by other means. Such light-inducible molecules include small molecule compounds, oligonucleotides, and proteins. For example, caged ecdysone, caged IPTG for use with the lac operon, caged toyocamycin for ribozyme-mediated gene expression, caged doxycycline for use with the Tet-on system, and caged Rapalog for light mediated FKBP/FRB dimerization have been developed (see, for example, Curr Opin Chem Biol. 16(3-4): 292-299 (2012)).

In some embodiments, the promoter is a radiation-inducible promoter, and the inducing condition is radiation, such as ionizing radiation. Radiation inducible promoters are also known in the art to control transgene expression. Alteration of gene expression occurs upon irradiation of cells. For example, a group of genes known as “immediate early genes” can react promptly upon ionizing radiation. Exemplary immediate early genes include, but are not limited to, Erg-1, p21/WAF-1, GADD45alpha, t-PA, c-Fos, c-Jun, NF-kappaB, and API. The immediate early genes comprise radiation responsive sequences in their promoter regions. Consensus sequences CC(A/T)₆GG have been found in the Erg-1 promoter, and are referred to as serum response elements or known as CArG elements. Combinations of radiation induced promoters and transgenes have been intensively studied and proven to be efficient with therapeutic benefits. See, for example, Cancer Biol Ther. 6(7):1005-12 (2007) and Chapter 25 of Gene and Cell Therapy: Therapeutic Mechanisms and Strategies, Fourth Edition CRC Press, Jan. 20, 2015.

In some embodiments, the promoter is a heat inducible promoter, and the inducing condition is heat. Heat inducible promoters driving transgene expression have also been widely studied in the art. Heat shock or stress protein (HSP) including Hsp90, Hsp70, Hsp60, Hsp40, Hsp10 etc. plays important roles in protecting cells under heat or other physical and chemical stresses. Several heat inducible promoters including heat-shock protein (HSP) promoters and growth arrest and DNA damage (GADD) 153 promoters have been attempted in pre-clinical studies. The promoter of human hsp70B gene, which was first described in 1985 appears to be one of the most highly-efficient heat inducible promoters. Huang et al reported that after introduction of hsp70B-EGFP, hsp70B-TNFalpha and hsp70B-IL12 coding sequences, tumor cells expressed extremely high transgene expression upon heat treatment, while in the absence of heat treatment, the expression of transgenes were not detected. Tumor growth was delayed significantly in the IL12 transgene plus heat-treated group of mice in vivo (Cancer Res. 60:3435 (2000)). Another group of scientists linked the HSV-tk suicide gene to hsp70B promoter and test the system in nude mice bearing mouse breast cancer. Mice whose tumor had been administered the hsp70B-HSVtk coding sequence and heat-treated showed tumor regression and a significant survival rate as compared to no heat treatment controls (Hum. Gene Ther. 11:2453 (2000)). Additional heat inducible promoters known in the art can be found in, for example, Chapter 25 of Gene and Cell Therapy: Therapeutic Mechanisms and Strategies, Fourth Edition CRC Press, Jan. 20, 2015. Any of the heat-inducible promoters discussed herein may be used to drive the expression of the therapeutic protein of the present invention.

In some embodiments, the promoter is inducible by a redox state. Exemplary promoters that are inducible by redox state include inducible promoter and hypoxia inducible promoters. For instance, Post D E et al developed hypoxia-inducible factor (HIF) responsive promoter, which specifically and strongly induce transgene expression in HIF-active tumor cells (Gene Ther. 8: 1801-1807 (2001); Cancer Res. 67: 6872-6881 (2007)).

In some embodiments, the promoter is inducible by the physiological state, such as an endogenous activation signal, of the modified therapeutic cell (e.g., modified immune cell). In some embodiments, wherein the modified therapeutic cell is a T cell, the promoter is a T cell activation-dependent promoter, which is inducible by the endogenous activation signal of the modified T cell. In some embodiments, the modified T cell is activated by an inducer, such as phorbol myristate acetate (PMA), ionomycin, or phytohaemagglutinin. In some embodiments, the modified T cell is activated by recognition of a tumor antigen on the tumor cells via the engineered receptor (such as CAR, TCR or TAC). In some embodiments, the T cell activation-dependent promoter is an IL-2 promoter. In some embodiments, the T cell activation-dependent promoter is an NFAT promoter. In some embodiments, the T cell activation-dependent promoter is a NFκB promoter.

The heterologous nucleic acid sequences(s) described herein can be present in a heterologous gene expression cassette, which comprises one or more protein-coding sequences and optionally one or more promoters. In some embodiments, the heterologous gene expression cassette comprises a single protein-coding sequence. In some embodiments, the heterologous gene expression cassette comprises two or more protein-coding sequences driven by a single promoter (i.e., polycistronic). In some embodiments, the heterologous gene expression cassette further comprises one or more regulatory sequences (such as 5′UTR, 3′UTR, enhancer sequence, IRES, transcription termination sequence), recombination sites, one or more selection markers (such as antibiotic resistance gene, reporter gene, etc.), signal sequence, or combinations thereof. In some embodiments, a first heterologous nucleic acid sequence encoding an ICL is fused to a second heterologous nucleic acid sequence encoding an engineered receptor via a third nucleic acid sequence encoding a self-cleavable linker, such as P2A, T2A, E2A, or F2A peptide. In some embodiments, the self-cleavable linker is a P2A peptide comprising the amino acid sequence of GSGATNFSLLKQAGDVEENPGP (SEQ ID NO: 11).

In some embodiments, the modified therapeutic cell comprises a nucleic acid comprising a first heterologous nucleic acid sequence encoding an ICL and a second heterologous nucleic acid sequence encoding an engineered receptor (e.g., a CAR), wherein the first heterologous nucleic acid sequence is fused to the second heterologous nucleic acid sequence via a third nucleic acid sequence encoding a 2A peptide (e.g., P2A peptide). In some embodiments, the first heterologous nucleic acid sequence is 5′ to the second heterologous nucleic acid sequence. In some embodiments, the first heterologous nucleic acid sequence is 3′ to the second heterologous nucleic acid sequence.

In some embodiments, the modified therapeutic cell comprises a heterologous nucleic acid sequence encoding the amino acid sequence of any one of SEQ ID NOs: 12-27, or a variant thereof having at least about 85% (e.g., at least about any one of 90%, 91%, 92%, 93%, 94%, 95%, 96%, 97%, 98%, 99%, or more) sequence identity to an amino acid sequence selected from the group consisting of SEQ ID NOs: 12-27.

In some embodiments, the modified therapeutic cell comprises a heterologous nucleic acid sequence encoding the amino acid sequence of SEQ ID NO: 12, or a variant thereof having at least about 85% (e.g., at least about any one of 90%, 91%, 92%, 93%, 94%, 95%, 96%, 97%, 98%, 99%, or more) sequence identity to the amino acid sequence of SEQ ID NO: 12.

In some embodiments, the modified therapeutic cell comprises a heterologous nucleic acid sequence encoding the amino acid sequence of SEQ ID NO: 20, or a variant thereof having at least about 85% (e.g., at least about any one of 90%, 91%, 92%, 93%, 94%, 95%, 96%, 97%, 98%, 99%, or more) sequence identity to the amino acid sequence of SEQ ID NO: 20.

In some embodiments, the heterologous nucleic acid sequence(s) are present in a vector. In some embodiments, the modified therapeutic cell comprises a vector comprising a first heterologous nucleic acid sequence encoding an ICL. In some embodiments, the modified therapeutic cell comprises a vector comprising a first heterologous nucleic acid sequence encoding an ICL and a second heterologous nucleic acid sequence encoding an engineered receptor.

In some embodiments, the vector is a viral vector. Examples of viral vectors include, but are not limited to, adenoviral vectors, adeno-associated virus vectors, lentiviral vector, retroviral vectors, vaccinia vector, herpes simplex viral vector, and derivatives thereof. Viral vector technology is well known in the art and is described, for example, in Sambrook et al. (2001, Molecular Cloning: A Laboratory Manual, Cold Spring Harbor Laboratory, New York), and in other virology and molecular biology manuals.

A number of viral based systems have been developed for gene transfer into mammalian cells. For example, retroviruses provide a convenient platform for gene delivery systems. The heterologous nucleic acid can be inserted into a vector and packaged in retroviral particles using techniques known in the art. The recombinant virus can then be isolated and delivered to the therapeutic cell (e.g., modified immune cell) in vitro or ex vivo. A number of retroviral systems are known in the art. In some embodiments, adenovirus vectors are used. In some embodiments, lentivirus vectors are used. In some embodiments, self-inactivating lentiviral vectors are used. For example, self-inactivating lentiviral vectors can be packaged with protocols known in the art. The resulting lentiviral vectors can be used to transduce a mammalian cell (such as human T cells) using methods known in the art.

In some embodiments, the vector is a non-viral vector, such as a plasmid, or an episomal expression vector.

In some embodiments, the vector is an expression vector. “Expression vector” is a construct that can be used to transform a selected host and provides for expression of a coding sequence in the selected host. Expression vectors can for instance be cloning vectors, binary vectors or integrating vectors. Expression comprises transcription of the nucleic acid molecule preferably into a translatable mRNA. Regulatory elements ensuring expression in eukaryotic cells are well known to those skilled in the art. In the case of eukaryotic cells they comprise normally promoters ensuring initiation of transcription and optionally poly-A signals ensuring termination of transcription and stabilization of the transcript. Examples of regulatory elements permitting expression in eukaryotic host cells are AOX1 or GAL1 promoter in yeast or the CMV-, SV40-, RSV-promoter (Rous sarcoma virus), CMV-enhancer, SV40-enhancer or a globin intron in mammalian and other animal cells. Furthermore, depending on the expression system used leader sequences capable of directing the polypeptide to a cellular compartment or secreting it into the medium may be added to the coding sequence of the recited nucleic acid sequence and are well known in the art. The leader sequence(s) is (are) assembled in appropriate phase with translation, initiation and termination sequences, and preferably, a leader sequence capable of directing secretion of translated protein, or a portion thereof, into the periplasmic space or extracellular medium. Optionally, the nucleic acid sequence can encode a fusion protein including an N-terminal identification peptide imparting desired characteristics, e.g., stabilization or simplified purification of expressed recombinant product. Suitable expression vectors are known in the art such as Okayama-Berg cDNA expression vector pcDV1 (Pharmacia), pEF-Neo, pCDM8, pRc/CMV, pcDNA1, pcDNA3 (Invitrogen), pEF-DHFR and pEF-ADA, (Raum et al., Cancer Immunol Immunother (2001) 50(3), 141-150) or pSPORT1 (GIBCO BRL).

### III. Methods of Treatment

One aspect of the present application relates to methods of treating a disease or condition (such as cancer) in an individual, comprising administering to the individual an effective amount of any one of the modified therapeutic cells described herein. The present application contemplates modified therapeutic cells that can be administered either alone or in any combination with another therapy, and in at least some aspects, together with a pharmaceutically acceptable carrier or excipient. In some embodiments, prior to administration, the modified therapeutic cells may be combined with suitable pharmaceutical carriers and excipients that are well known in the art.

In some embodiments, there is provided a method of treating a disease or condition (e.g., cancer) in an individual (e.g., human), comprising administering to the individual an effective amount of a pharmaceutical composition comprising a modified therapeutic cell (e.g., a modified immune cell) and a pharmaceutically acceptable carrier, wherein the modified therapeutic cell comprises a first heterologous nucleic acid sequence encoding an ICL, wherein the therapeutic cell expresses an MHC molecule. In some embodiments, the therapeutic cell expresses an MHC class I molecule. In some embodiments, the therapeutic cell expresses an MHC class II molecule. In some embodiments, the therapeutic cell expresses both an MHC class I molecule and an MHC class II molecule. In some embodiments, the β2-microglobulin (B2M) gene of the therapeutic cell is not genetically modified. In some embodiments, the MHC genes of the therapeutic cell are not genetically modified. In some embodiments, the modified therapeutic cell is allogeneic.

In some embodiments, there is provided a method of treating a disease or condition (e.g., cancer) in an individual (e.g., human), comprising administering to the individual an effective amount of a pharmaceutical composition comprising a modified immune cell and a pharmaceutically acceptable carrier, wherein the modified immune cell comprises a first heterologous nucleic acid sequence encoding an ICL selected from the group consisting of PD-L1, CD155, CD112, FGL1, galectin-9, CD47, B7H3 and B7H4, wherein the immune cell expresses an MHC molecule. In some embodiments, the immune cell expresses an MHC class I molecule. In some embodiments, the immune cell expresses an MHC class II molecule. In some embodiments, the immune cell expresses both an MHC class I molecule and an MHC class II molecule. In some embodiments, the β2-microglobulin (B2M) gene of the immune cell is not genetically modified. In some embodiments, the MHC genes of the immune cell are not genetically modified. In some embodiments, the immune cell is selected from the group consisting of cytotoxic T cell, helper T cell, NK cell, NK-T cell, αβ T cell, γδ T cell, tumor-infiltrating T cell, dendritic cell (DC)-activated T cell, and peripheral blood mononuclear cell (PBMC). In some embodiments, the modified therapeutic cell is a T cell. In some embodiments, the modified therapeutic cell is a γδ T cell. In some embodiments, the modified immune cell is allogeneic.

In some embodiments, there is provided a method of treating a disease or condition (e.g., cancer) in an individual (e.g., human), comprising administering to the individual an effective amount of a pharmaceutical composition comprising a modified stem cell and a pharmaceutically acceptable carrier, wherein the modified stem cell comprises a first heterologous nucleic acid sequence encoding an ICL selected from the group consisting of PD-L1, CD155, CD112, FGL1, galectin-9, CD47, B7H3 and B7H4, wherein the stem cell expresses an MHC molecule. In some embodiments, the stem cell expresses an MHC class I molecule. In some embodiments, the stem cell expresses an MHC class I molecule. In some embodiments, the stem cell expresses both an MHC class I molecule and an MHC class II molecule. In some embodiments, the β2-microglobulin (B2M) gene of the stem cell is not genetically modified. In some embodiments, the MHC genes of the stem cell are not genetically modified. In some embodiments, the modified stem cell is an HSC. In some embodiments, the modified stem cell is an ESC. In some embodiments, the modified stem cell is allogeneic.

In some embodiments, there is provided a method of treating a disease or condition (e.g., cancer) in an individual (e.g., human), comprising administering to the individual an effective amount of a pharmaceutical composition comprising a modified immune cell and a pharmaceutically acceptable carrier, wherein the modified immune cell comprises a first heterologous nucleic acid sequence encoding an ICL selected from the group consisting of PD-L1, CD155, CD112, FGL1, galectin-9, CD47, B7H3 and B7H4, and a second heterologous nucleic acid sequence encoding an engineered receptor, wherein the immune cell expresses an MHC molecule. In some embodiments, the immune cell expresses an MHC class I molecule. In some embodiments, the immune cell expresses an MHC class II molecule. In some embodiments, the immune cell expresses both an MHC class I molecule and an MHC class II molecule. In some embodiments, the β2-microglobulin (B2M) gene of the immune cell is not genetically modified. In some embodiments, the MHC genes of the immune cell are not genetically modified. In some embodiments, the engineered receptor is selected from the group consisting of a chimeric antigen receptor (CAR), a recombinant T cell receptor (TCR), a T-cell antigen coupler (TAC) receptor and a TCR fusion protein (TFP). In some embodiments, the engineered receptor targets a tumor antigen selected from the group consisting of CD19, BCMA, NY-ESO-1, VEGFR2, MAGE-A3, CD20, CD22, CD33, CD38, CEA, EGFR, GD2, HER2, IGF1R, mesothelin, PSMA, ROR1, WT1 and combinations thereof. In some embodiments, the modified immune cell is allogeneic.

In some embodiments, there is provided a method of treating a disease or condition (e.g., cancer) in an individual (e.g., human), comprising administering to the individual an effective amount of a pharmaceutical composition comprising a CAR-T cell and a pharmaceutically acceptable carrier, wherein the CAR-T cell comprises a first heterologous nucleic acid sequence encoding an ICL selected from the group consisting of PD-L1, CD155, CD112, FGL1, galectin-9, CD47, B7H3 and B7H4, and a second heterologous nucleic acid sequence encoding a CAR, wherein the immune cell expresses an MHC molecule. In some embodiments, the CAR-T cell expresses an MHC class I molecule. In some embodiments, the CAR-T cell expresses an MHC class II molecule. In some embodiments, the CAR-T cell expresses both an MHC class I molecule and an MHC class II molecule. In some embodiments, the β2-microglobulin (B2M) gene of the CAR-T cell is not genetically modified. In some embodiments, the MHC genes of the CAR-T cell are not genetically modified. In some embodiments, the CAR targets a tumor antigen selected from the group consisting of CD19, BCMA, NY-ESO-1, VEGFR2, MAGE-A3, CD20, CD22, CD33, CD38, CEA, EGFR, GD2, HER2, IGF1R, mesothelin, PSMA, ROR1, WT1 and combinations thereof. In some embodiments, the CAR is an anti-BCMA CAR. In some embodiments, the CAR is an anti-CD19 CAR. In some embodiments, the ICL is PD-L1. In some embodiments, the CAR-T cell is allogeneic.

In some embodiments, there is provided a method of treating a disease or condition (e.g., cancer) in an individual (e.g., human), comprising administering to the individual an effective amount of a pharmaceutical composition comprising a CAR-T cell and a pharmaceutically acceptable carrier, wherein the CAR-T cell comprises a first heterologous nucleic acid sequence encoding PD-L1, and a second heterologous nucleic acid sequence encoding a CAR targeting BCMA, wherein the immune cell expresses an MHC molecule. In some embodiments, the CAR-T cell expresses an MHC class I molecule. In some embodiments, the CAR-T cell expresses an MHC class II molecule. In some embodiments, the CAR-T cell expresses both an MHC class I molecule and an MHC class H molecule. In some embodiments, the β2-microglobulin (B2M) gene of the CAR-T cell is not genetically modified. In some embodiments, the MHC genes of the CAR-T cell are not genetically modified. In some embodiments, the CAR-T cell is allogeneic. In some embodiments, the CAR is BSF17 CAR. In some embodiments, the CAR comprises an amino acid sequence of SEQ ID NO: 10, or a variant thereof having at least about 85% (e.g., at least about any one of 90%, 91%, 92%, 93%, 94%, 95%, 96%, 97%, 98%, 99%, or more) sequence identity to SEQ ID NO: 10. In some embodiments, the PD-L1 comprises an amino acid sequence of SEQ ID NO. 1, or a variant thereof having at least about 85% (e.g., at least about any one of 90%, 91%, 92%, 93%, 94%, 95%, 96%, 97%, 98%, 99%, or more) sequence identity to SEQ ID NO: 1. In some embodiments, the CAR-T cell comprises a nucleic acid sequence encoding an amino acid sequence of SEQ ID NO: 12, or a variant thereof having at least about 85% (e.g., at least about any one of 90%, 91%, 92%, 93%, 94%, 95%, 96%, 97%, 98%, 99%, or more) sequence identity to SEQ ID NO. 12.

The methods described herein are suitable for treating diseases or conditions such as cancer, autoimmune diseases, or infectious diseases. The methods described herein can be used to treat a variety of cancers, including both solid cancer and liquid cancer. In some embodiments, the cancer is selected from the group consisting of leukemia, lymphoma, melanoma, breast cancer, lung cancer, liver cancer, gastric cancer, colon cancer, bone cancer, brain cancer, pancreatic cancer, and ovarian cancer. The methods are applicable to cancers of all stages, including early stage cancer, non-metastatic cancer, primary cancer, advanced cancer, locally advanced cancer, metastatic cancer, or cancer in remission. The methods described herein may be used as a first therapy, second therapy, third therapy, or combination therapy with other types of cancer therapies known in the art, such as chemotherapy, surgery, hormone therapy, radiation, gene therapy, immunotherapy, bone marrow transplantation, stem cell transplantation, targeted therapy, cryotherapy, ultrasound therapy, photodynamic therapy, radio-frequency ablation or the like, in an adjuvant setting or a neoadjuvant setting (i.e., the method may be carried out before the primary/definitive therapy). In some embodiments, the method is used to treat an individual who has previously been treated. In some embodiments, the cancer has been refractory to prior therapy. In some embodiments, the method is used to treat an individual who has not previously been treated. In some embodiments, the method is for treating blood cancer, such as leukemia or lymphoma, including plasmacytoma and myeloma.

The effective amount of the modified therapeutic cells administered in the methods described herein will depend upon a number of factors, such as the particular type and stage of disease or condition (e.g., cancer) being treated, the route of administrations, the activity of the engineered receptors, and the like. Appropriate dosage regimen can be determined by a physician based on clinical factors, including the patient's size, body surface area, age, the particular compound to be administered, sex, time and route of administration, general health, and other drugs being administered concurrently. In some embodiments, the effective amount of the pharmaceutical composition is below the level that induces a toxicological effect (i.e., an effect above a clinically acceptable level of toxicity) or is at a level where a potential side effect can be controlled or tolerated when the pharmaceutical composition is administered to the individual. In some embodiments, the effective amount of the pharmaceutical composition comprises about 10³ to about 10⁹ modified therapeutic cells/kg.

In some embodiments, the pharmaceutical composition is administered for a single time (e.g. bolus injection). In some embodiments, the pharmaceutical composition is administered for multiple times (such as any of 2, 3, 4, 5, 6, or more times). When the pharmaceutical composition is administered for multiple times, each administration may use the same or different routes and may take place at the same site or at alternative sites. The pharmaceutical composition may be administered at a suitable frequency, such as from daily to once per year. The optimal dosage and treatment regime for a particular patient can readily be determined by one skilled in the art of medicine by monitoring the patient for signs of disease and adjusting the treatment accordingly.

In some embodiments, the individual to be treated is a mammal. Examples of mammals include, but are not limited to, humans, monkeys, rats, mice, hamsters, guinea pigs, dogs, cats, rabbits, pigs, sheep, goats, horses, cattle and the like. In some embodiments, the individual is a human.

The modified therapeutic cells are suitable in both autologous cell therapy and allogeneic cell therapy. In some embodiments, the modified therapeutic cell is allogeneic for the individual to be treated. In some embodiments, the modified therapeutic cell has matching MHC allotypes as the individual to be treated. In some embodiments, the modified therapeutic cell has mismatching MHC allotypes as the individual to be treated.

The genetic loci involved in the rejection of foreign organs are known as the major histocompatibility complex (MHC), and highly polymorphic cell surface molecules are encoded by the MHC. The human MHC is called the HLA (Human Leukocyte Antigen). Graft rejection is associated with the development of antibodies against allogeneic leukocytes.

MHC class I molecules are expressed on the surface of almost all nucleated cells. Class I HLA genes include HLA-A, HLA-B and HLA-C, which encode the heavy chains of class I molecules. Minor class I HLA genes include HLA-E, HLA-F and HLA-G. β2-microglobulin binds with major and minor gene subunits to produce an HLA class I heterodimer.

MHC class II molecules are expressed on B lymphocytes, antigen-presenting cells (e.g., monocytes, macrophages, and dendritic cells), and activated T cells. Class II HLA genes include HLA-DP (i.e., HLA-DPA1 encoding alpha chain, and HLA-DPB1 encoding beta chain), HLA-DQ (i.e., HLA-DQA1 encoding alpha chain, and HLA-DQB1 encoding beta chain), and HLA-DR (i.e., HLA-DRA encoding alpha chain, and HLA-DRB1, DRB3, DRB4 and DRB5 encoding beta chain).

HLA genes are highly polymorphic, and each HLA gene has many different alleles. HLA typing, HLA antibody screening and crossmatching can be carried out to assess whether a modified therapeutic cell has mismatched HLA allele(s) compared to those of the individual being treated, as recognition of foreign HLA by the individual's T cells could trigger an immune response against adoptively transferred cells. Methods for HLA typing, antibody screening and crossmatching are known in the art. See, for example, Choo, Yonsei Medical Journal, 48(1): 11-23 (2007); and Althaf et al., World Journal of Transplantation, 2017, 7(6): 339-348, which are incorporated herein by reference in their entirety.

HLA typing can be done using serologic or molecular typing methods. In a serologic HLA typing assay, a tray containing sera with antibodies to a multitude of known HLA alleles is used. For typing, recipient lymphocytes are introduced into the tray wells contacting sera, complement and dye. In tray wells where antibodies can bind to the antigens on the surface of lymphocytes, complement is activated. This results in complement pathways triggered resulting in cell death, ultimately allowing the dye to enter the cell. Tray wells with significant cell death are then identified under phase contrast microscopy. Through a process of comparison and elimination of positive wells, the HL A type is assigned. Peripheral blood lymphocytes (PBLs) can be used for serologic typing of HLA-A, HLA-B and HLA-C. B cells isolated from PBLs can be used for serologic typing of HLA class II, such as HLA-DRB1. Molecular HLA typing can be carried out using PCR-based assays such as sequence-specific oligonucleotide probes (SSOP) or sequence-specific primer (SSP) method, or by DNA sequencing.

In some embodiments, one or more HLA alleles of the individual have mismatching allotypes compared to those of the modified therapeutic cell. In some embodiments, the mismatching alleles are for one or more class I HLA genes. In some embodiments, the mismatching alleles are for one or more class II HLA genes. In some embodiments, no more than any one of 8, 7, 6, 5, 4, 3, 2, or 1 alleles of HLA-A, HLA-B, HLA-C and HLA-DRB1 of the individual have mismatching allotypes compared to those of the modified therapeutic cell. In some embodiments, all tested HLA alleles (e.g., HLA-A, HLA-B, HLA-C and/or HLA-DRB1) of the individual have mismatching allotypes compared to those of the modified therapeutic cell. In some embodiments, all tested HLA alleles (e.g., HLA-A, HLA-B, HLA-C and/or HLA-DRB1) of the individual have matching allotypes compared to those of the modified therapeutic cell.

In some embodiments, HLA antibody screening is carried out prior to the treatment methods. Preformed HLA antibodies can be detected by testing the individual's serum against a panel of lymphocytes with known HLA types. Complement-mediated microlymphocytotoxicity technique or anti-human globulin (AHG) enhancement method can be used for HLA antibody screening. The results are expressed as the percentage of the panel cells that are reactive, which is referred to as percentage panel reactive antibody (% PRA). In some embodiments, the individual has no more than about any one of 90%, 80%, 70%, 60%, 50%, 40%, 30%, 25%, or less PRA.

In some embodiments, crossmatching is performed between the individual and the modified therapeutic cell. In some embodiments, the individual is crossmatch-negative with respect to the modified therapeutic cell. In some embodiments, the individual is crossmatch-positive with respect to the modified therapeutic cell. In some embodiments, the individual has pre-formed antibodies against one or more HLA alleles of the modified therapeutic cell.

In some embodiments, the method reduces undesired immune response associated with the modified therapeutic cell compared to a method using a therapeutic cell that does not comprise the first heterologous nucleic acid sequence encoding the ICL. In some embodiments, the method reduces undesired immune response associated with the modified therapeutic cell in the individual by at least about any one of 10%, 20%, 30%, 40%, 50%, 60%, 70%, 80%, 90% or more. In some embodiments, the undesired immune response comprises HvGD. In some embodiments, the method prevents HvGD by at least about any one of 10, 20, 30, 40, 50, 60, or more days. In some embodiments, the undesired immune response is mediated by allogeneic αβT cells in the individual receiving the therapeutic cell. In some embodiments, the undesired immune response is mediated by allogeneic NK cells in the individual receiving the therapeutic cell. In some embodiments, the method reduces proliferation of allogeneic αβT cells in the individual receiving the therapeutic cell by at least about any one of 10%, 20%, 30%, 40%, 50%, 60%, 70%, 80%, 90% or more, compared to a method using a therapeutic cell that does not comprise the first heterologous nucleic acid sequence encoding the ICL. In some embodiments, the method reduces proliferation of allogeneic NK cells in the individual receiving the therapeutic cell by at least about any one of 10%, 20%, 30%, 40%, 50%, 60%, 70%, 80%, 90% or more, compared to a method using a therapeutic cell that does not comprise the first heterologous nucleic acid sequence encoding the ICL. In some embodiments, the undesired immune response comprises graft rejection. In some embodiments, the method prevents graft rejection by at least about any one of 10, 20, 30, 40, 50, 60 or more days. In some embodiments, the undesired immune response comprises anti-drug-antibody (ADA) against the modified therapeutic cell. In some embodiments, the method reduces ADA against the modified therapeutic cell by at least about any one of 10%, 20%, 30%, 40%, 50%, 60%, 70%, 80%, 90%, or more. In some embodiments, the method induces immune tolerance towards the modified therapeutic cell in the individual compared to a method using a therapeutic cell that does not comprise the first heterologous nucleic acid sequence encoding the ICL. In some embodiments, the method reduces or inhibits NK cell-mediated clearance of the modified therapeutic cell. In some embodiments, the method reduces or inhibits T cell-mediated clearance of the modified therapeutic cell. In some embodiments, the method reduces or inhibits both NK cell-mediated and T cell-mediated clearance of the modified therapeutic cell.

In some embodiments, there is provided a method of reducing graft rejection against allogeneic therapeutic cells in an individual in need thereof, comprising administering to the individual an effective amount of the allogeneic therapeutic cells, wherein the allogeneic therapeutic cells comprise a first heterologous nucleic acid sequence encoding an ICL, and wherein the therapeutic cells express an MHC molecule. In some embodiments, the ICL is selected from the group consisting of PD-L1, CD155, CD112, FGL1, galectin-9, CD47, B7H3 and B7H4. In some embodiments, the therapeutic cells are immune cells, such as cytotoxic T cell, helper T cell, NK cell, NK-T cell, ca T cell, γδ T cell, tumor-infiltrating T cell, dendritic cell (DC)-activated T cell, peripheral blood mononuclear cell (PBMC), or combinations thereof. In some embodiments, the therapeutic cells are stem cells, such as HSC or ESC. In some embodiments, the therapeutic cells further comprise a second heterologous nucleic acid sequence encoding an engineered receptor, such as a CAR, a recombinant TCR, a TAC receptor, and/or a TFP. In some embodiments, the therapeutic cells are CAR-T cells. In some embodiments, the ICL is expressed at an increased level compared to an unmodified therapeutic cell. In some embodiments, the ICL is PD-L1. In some embodiments, the therapeutic cell is a CAR-T cell.

### Pharmaceutical Compositions

In some embodiments, there is provided a composition comprising any one of the modified therapeutic cells described herein. The composition may comprise any number of the modified therapeutic cell. In some embodiments, the composition compress a single copy of the modified therapeutic cell. In some embodiments, the composition comprises at least about any of 1, 10, 100, 1000, 10⁴, 10⁵, 10⁶, 10⁷, 10⁸ or more copies of the modified therapeutic cells. In some embodiments, there is provided a pharmaceutical composition comprising an effective amount of modified therapeutic cell (such as allogeneic CAR-T cell), and a pharmaceutically acceptable carrier.

Also provided are the compositions for use in any one of the methods described herein, and use of the compositions in preparation of a medicament for any one of the methods described herein.

“Carriers” as used herein include pharmaceutically acceptable carriers, excipients, or stabilizers which are nontoxic to the cells or individual being exposed thereto at the dosages and concentrations employed. Often the physiologically acceptable carrier is an aqueous pH buffered solution. Examples of suitable pharmaceutical carriers are well known in the art and include phosphate buffered saline solutions, water, emulsions, such as oil/water emulsions, various types of wetting agents, sterile solutions, etc. Acceptable carriers, excipients, or stabilizers are nontoxic to recipients at the dosages and concentrations employed.

Pharmaceutical compositions comprising such carriers can be formulated by well-known conventional methods. The solvent or diluent is preferably isotonic, hypotonic or weakly hypertonic and has a relatively low ionic strength. Representative examples include sterile water, physiological saline (e.g., sodium chloride), Ringer's solution, glucose, trehalose or saccharose solutions, Hank's solution, and other aqueous physiologically balanced salt solutions (see, for example, the most current edition of Remington: The Science and Practice of Pharmacy, A. Gennaro, Lippincott, Williams & Wilkins).

The pharmaceutical compositions described herein may be administered via any suitable routes. In some embodiments, the pharmaceutical composition is administered parenterally, transdermally (into the dermis), intraluminally, intra-arterially (into an artery), intramuscularly (into muscle), intrathecally or intravenously. In some embodiments, the pharmaceutical composition is administered subcutaneously (under the skin). In some embodiments, the pharmaceutical composition is administered intravenously. In some embodiments, the pharmaceutical composition is administered to the individual via infusion or injection. In some embodiments, the pharmaceutical composition is administered directly to the target site, e.g., by biolistic delivery to an internal or external target site or by catheter to a site in an artery. In some embodiments, the pharmaceutical composition is administered locally, e.g., intratumorally. Administrations may use conventional syringes and needles or any compound or device available in the art capable of facilitating or improving delivery of the active agent(s) in the subject.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringers dextrose), and the like. Preservatives and other additives may also be present such as, for example, antimicrobials, anti-oxidants, chelating agents, and inert gases and the like. In addition, the pharmaceutical composition of the present disclosure might comprise proteinaceous carriers, like, e.g., serum albumin or immunoglobulin, preferably of human origin.

In some embodiments, the pharmaceutical composition is suitably buffered for human use. Suitable buffers include without limitation phosphate buffer (e.g. PBS), bicarbonate buffer and/or Tris buffer capable of maintaining a physiological or slightly basic pH (e.g., from approximately pH 7 to approximately pH 9). In some embodiments, the pharmaceutical composition can also be made to be isotonic with blood by the addition of a suitable tonicity modifier, such as glycerol.

In some embodiments, the pharmaceutical composition is contained in a single-use vial, such as a single-use sealed vial. In some embodiments, the pharmaceutical composition is contained in a multi-use vial. In some embodiments, the pharmaceutical composition is contained in bulk in a container.

In some embodiments, the pharmaceutical composition must meet certain standards for administration to an individual. For example, the United States Food and Drug Administration has issued regulatory guidelines setting standards for cell-based immunotherapeutic products, including 21 CFR 610 and 21 CFR 610.13. Methods are known in the art to assess the appearance, identity, purity, safety, and/or potency of pharmaceutical compositions. In some embodiments, the pharmaceutical composition is substantially free of extraneous protein capable of producing allergenic effects, such as proteins of an animal source used in cell culture other than the therapeutic cells. In some embodiments, “substantially free” is less than about any of 10%, 5%, 1%, 0.1%, 0.01%, 0.001%, 1 ppm or less of total volume or weight of the pharmaceutical composition. In some embodiments, the pharmaceutical composition is prepared in a GMP-level workshop. In some embodiments, the pharmaceutical composition comprises less than about 5 EU/kg body weight/hr of endotoxin for parenteral administration. In some embodiments, at least about 70% of the therapeutic cells in the pharmaceutical composition are alive for intravenous administration. In some embodiments, the pharmaceutical composition has a “no growth” result when assessed using a 14-day direct inoculation test method as described in the United States Pharmacopoeia (USP). In some embodiments, prior to administration of the pharmaceutical composition, a sample including both the therapeutic cells and the pharmaceutically acceptable excipient should be taken for sterility testing approximately about 48-72 hours prior to the final harvest (or coincident with the last re-feeding of the culture). In some embodiments, the pharmaceutical composition is free of mycoplasma contamination. In some embodiments, the pharmaceutical composition is free of detectable microbial agents. In some embodiments, the pharmaceutical composition is free of communicable disease agents, such as HIV type I, HIV type II, HBV, HCV, Human T-lymphotropic virus, type I; and Human T-lymphotropic virus, type II.

### IV. Kits and Articles of Manufacture

Also provided are kits, unit dosages, and articles of manufacture comprising any one of the modified therapeutic cells described herein.

In some embodiments, a kit is provided which contains any one of the pharmaceutical compositions described herein and preferably provides instructions for its use. In some embodiments, there is provided a kit comprising: (a) any one of the modified therapeutic cells described herein; and (b) instructions for use in any one of the methods described herein. In some embodiments, the modified therapeutic cells are allogeneic therapeutic cells. In some embodiments, the modified therapeutic cells are modified immune cells.

In some embodiments, there is provided a kit comprising: (a) a composition comprising allogeneic CAR-T cells comprising a first heterologous nucleic acid sequence encoding an ICL, wherein the CAR-T cells express a MHC molecule; and (b) instructions for treating a disease or condition (e.g., cancer) in an individual in need thereof. In some embodiments, the allogeneic CAR-T cells are universal CAR-T cells. In some embodiments, the allogeneic CAR-T cells target BCMA, or CD19. In some embodiments, the allogeneic CAR-T cells are not genetically modified to reduce immunogenicity of the allogeneic cells in the individual. In some embodiments, the MHC genes (e.g., the B2M gene) of the allogeneic CAR-T cells are not genetically modified. In some embodiments, the allogeneic CAR-T cells have no genetic modification except for the CAR or the ICL. In some embodiments, the ICL is selected from the group consisting of PD-L1, CD155, CD112, FGL1, galectin-9, CD47, B7H3 and B714.

In some embodiments, the kit further comprises one or more lymphodepletion agents. In some embodiments, the kit further comprises fludarabine and cyclophosphamide. In some embodiments, the kit, in addition to the modified therapeutic cells, further comprises a second cancer therapy, such as chemotherapy, hormone therapy, and/or immunotherapy. The kit(s) may be tailored to a particular cancer for an individual and comprise respective second cancer therapies for the individual.

The kits may contain one or more additional components, such as containers, reagents, culturing media, inducers, cytokines, buffers, antibodies, and the like to allow propagation or induction of the modified therapeutic cells. The kits may also contain a device for administration of the pharmaceutical composition.

The kits of the present application are in suitable packaging. Suitable packaging includes, but is not limited to, vials, bottles, jars, flexible packaging (e.g., sealed Mylar or plastic bags), and the like. Kits may optionally provide additional components such as buffers and interpretative information. The present application thus also provides articles of manufacture, which include vials (such as sealed vials), bottles, jars, flexible packaging, and the like. Some components of the kits may be packaged either in aqueous media or in lyophilized form.

The article of manufacture can comprise a container and a label or package insert on or associated with the container. Suitable containers include, for example, bottles, vials, syringes, etc. The containers may be formed from a variety of materials such as glass or plastic. Generally, the container holds a composition, which is effective for treating a disease, or disorder (such as cancer) described herein, and may have a sterile access port (for example, the container may be an intravenous solution bag or a vial having a stopper pierceable by a hypodermic injection needle). The label or package insert indicates that the composition is used for treating the particular condition in an individual. The label or package insert will further comprise instructions for administering the composition to the individual. The label may indicate directions for reconstitution and/or use. The container holding the pharmaceutical composition may be a multi-use vial, which allows for repeat administrations (e.g., from 2-6 administrations). Package insert refers to instructions customarily included in commercial packages of therapeutic products that contain information about the indications, usage, dosage, administration, contraindications and/or warnings concerning the use of such therapeutic products. Additionally, the article of manufacture may further comprise a second container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

The kits or article of manufacture may include multiple unit doses of the pharmaceutical composition and instructions for use, packaged in quantities sufficient for storage and use in pharmacies, for example, hospital pharmacies and compounding pharmacies.

### EXAMPLES

The examples and exemplary embodiments below are intended to be purely exemplary of the invention and should therefore not be considered to limit the invention in any way. The following examples and detailed description are offered by way of illustration and not by way of limitation.

### Example 1: Plasmid Construction and Virus Production

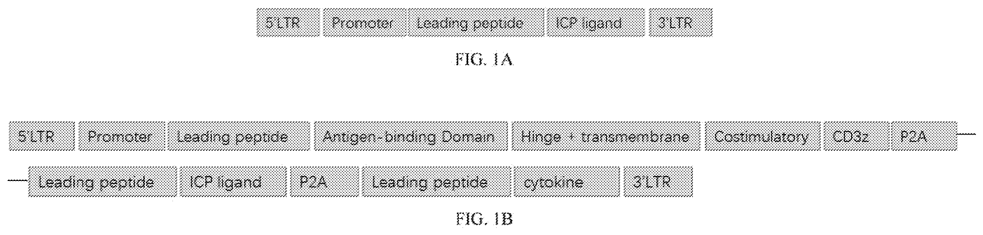

Constructs encoding an immune checkpoint ligand (ICL), such as PD-L1, CD155, CD112, FGL1, galectin-9, CD47, B7H3 or B7H4, a chimeric antigen receptor (CAR) with a co-expressed ICL (also referred herein as “armored CAR”), or a control CAR that does not have a co-expressed ICL (also referred herein as “unarmored CAR”) were designed as shown in FIGS. 1A and 1B. SEQ ID NOs: 1-8 are exemplary sequences of the ICLs. SEQ ID NOs: 9-10 are exemplary sequences of CARs. SEQ ID NOs: 12-27 are exemplary sequences of ICL-armored CARs. Lentiviral vector pLVX-Puro (Clontech #632164) was modified by replacing the original promoter with human elongation factor 1α promoter (hEF1a) and by removing the puromycin resistance gene with EcoR1 and BamHI to provide PLVX-EF1A. The constructs were each cloned into PLVX-EF1A, which was then subject to the lentivirus packaging procedure as described below.

To generate viral particles comprising nucleic acids of the target constructs above, lentivirus packaging plasmid mixture including pMDLg/pRRE (Addgene #11251), pRSV-Rev (Addgene #11253), and pMD2.G (Addgene #11259) were pre-mixed with a PLVX-EF1A vector (including a target construct) at a pre-optimized ratio with polyetherimide (PEI), mixed properly, and incubated at room temperature for 5 minutes. The transfection mix was added dropwise to 293-T cells and mixed gently. Transfected 293-T cells were incubated overnight at 37° C. and 5% CO₂. Twenty-four hours post-transfection, supernatants were collected and centrifuged at 4° C., 500 g for 10 minutes to remove any cellular debris. Centrifuged supernatants were filtered through a 0.45 m PES filter to concentrate the viral supernatants post ultracentrifugation. After centrifugation, the supernatants were carefully discarded and the virus pellets were rinsed with pre-chilled DPBS. The concentration of virus was measured. Virus was aliquoted and stored at −80° C. Viral titers were determined by functional transduction of a standard T cell line.

The following examples demonstrated efficacy of CAR-T cells expressing PD-L1 as a representative immune checkpoint ligand for treating multiple myeloma and B cell malignancies as exemplary indications, which are targeted by the CAR constructs via BCMA and CD-19 respectively. Therefore, viruses encoding said designs (i.e., corresponding to SEQ ID NOs: 9, 10, 12 and 20) were selected for transduction of both a PT and γδT cells.

### Example 2: T Cell Transduction and FACS Analysis of Transduced T Cells αβ T Cells Transduction

αβ leukocytes were collected in R10 medium, and then mixed with 0.9% NaCl solution at a 1:1 (v/v) ratio 3 mL, of Lymphoprep medium was added to a 15 mL centrifuge tube and slowly layered to form 6 mL of diluted lymphocyte mix. The lymphocyte mix was centrifuged at 800 g for 30 minutes without brakes at 20° C. Lymphocyte buffy coat was then collected with a 200 μL pipette. The harvested fraction was diluted at least 6 fold using 0.9% NaCl or R10 to reduce the density of the solution before further centrifugation at 250 g for 10 minutes at 20° C. The supernatant was aspirated completely, and 10 mL of R10 was added to the cell pellet. The mixture was further centrifuged at 250 g for 10 minutes at 20° C. The supernatant was then aspirated. Two milliliters R10 was pre-warmed at 37° C. and supplemented with 100 J/mL IL-2, which was added to the cell pellet to re-suspend the cell pellet gently. Cells were quantified and the PBMC sample was ready for experimentation. Human T cells were purified from PBMCs using Miltenyi Pan T cell isolation kit (Cat #130-096-535).

The prepared αβ T cells were subsequently pre-activated for 48 hours with human T cell Activation/Expansion kit (Milteny #130-091-441) by using one loaded anti-Biotin MACSiBead Particle per two cells (bead-to-cell ratio 1:2).

The pre-activated αβ T cells were collected and re-suspended in 1640 medium containing 300 IU/mL IL-2. Lentiviral vectors encoding the constructs of Example 1 were each diluted to MOI=5 with the same medium and used to infect 10⁶ pre-activated αβ T cells. The pre-activated T cells were transduced with lentivirus stock in the presence of 8 μg/ml polybrene with centrifugation at 1000 g, 32° C. for 1 h. The transduced cells were then transferred to the cell culture incubator for transgene expression under suitable conditions. The following day, the transduced cells were centrifuged and replaced with fresh media. Cell density was measured every other day, and fresh media were added to continue the expansion.

### γδ T Cells Transducdon

PBMCs were isolated by density centrifugation (lymphoprep) from leukapheresis material and cryopreserved. PBMCs were resuscitated and activated with zoledronic acid (5 μM) in cell culture media AIM-V supplemented with IL-2 (1000 IU/ml) and 5% human AB serum and kept in a humidified chamber (37° C., 5% CO₂). Forty-eight hours post-activation, cells were transduced with lentiviral vectors encoding the constructs described in Example 1 at an MOI of 5 with 5 pg/ml polybrene. Such transduction procedure was repeated the next day followed by replenishment of fresh media containing IL-2 (1000 IU/ml) the day after the second transduction. Cells were cultured in AIM-V medium supplemented with IL-2 (1000 IU/ml) in a humidified chamber with periodical change of media as determined by the pH of the culture media for further expansion. Cells were harvested 10 days post-transduction and the total number, purity and transduction efficiency were determined. Cells were further enriched with a negative TCRγ/δ+ T cell isolation kit (Miltenyi Biotec) before future applications or cryopreserved.

### Example 3: Quantification of Transgene Expression

On day 7 post-transduction, cells were evaluated for expression of chimeric antigen receptors and ICL armors of the indicated constructs of Example 1 by flow cytometry. An aliquot of cells were collected from the culture, washed, pelleted, and re-suspended in diluted antibodies (eBioscience Anti-human TCR beta PE, anti-CAR Ab or anti-ICP ligand antibodies) at a dilution factor of 100 in 50-100 μl of PBS+0.5% FBS per sample. Cell were incubated at 4° C. for 30 minutes. Viability dye eFluor780 or SYTOX Blue viability stain was then added according to manufacturer's instructions. Cells were washed twice in PBS post incubation and resuspended in 100 to 200 μl PBS for analysis. The mean fluorescence of the system was quantified by flow cytometry.

For ICL staining, cells were stained with PE-labeled antibodies (eBioscience, PD-L1, Cat #12-5983-42). For anti-BCMA CAR-T staining, cells were stained with Alexa Fluor 488-labeled mouse-anti-camel sdAb antibodies (Genscript). For anti-CD19 CAR-T staining, cells were stained with Alexa Fluor 488-labeled human CD19 protein (Genscript). Flow cytometry analysis was performed using FlowJo (Tree Star, Inc.). Staining of cells expressing other ICL ligands can be carried out with PE-labeled antibodies: eBioscience. CD155, Cat #12-1550-41; CD112, Cat #12-1121-82; Galectin-9, Cat #25-9211-82; FGL1, Cat #PA5-30030; CD47 Cat #25-0471-80; B7H3, Cat #12-5973-80; and B7H4, Cat #12-5949-41.

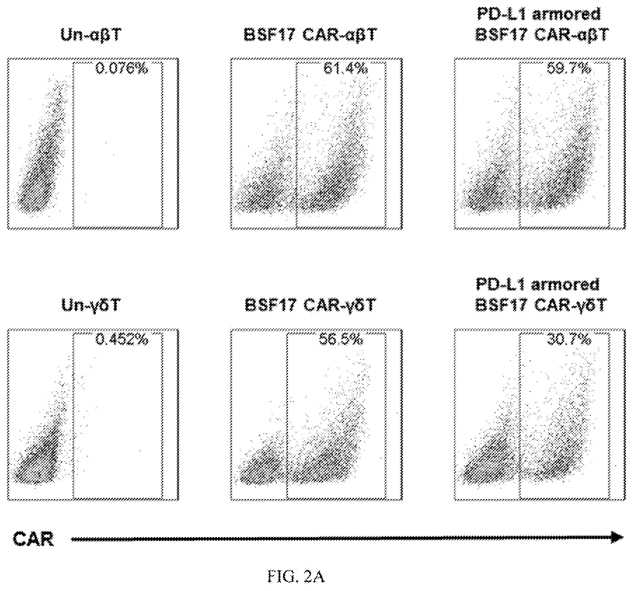

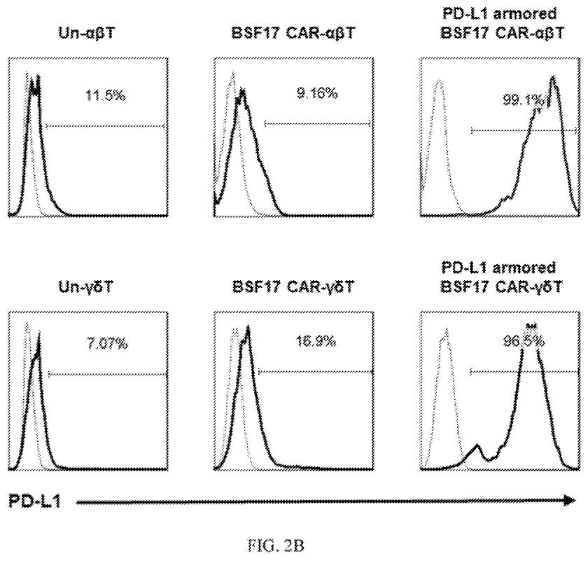

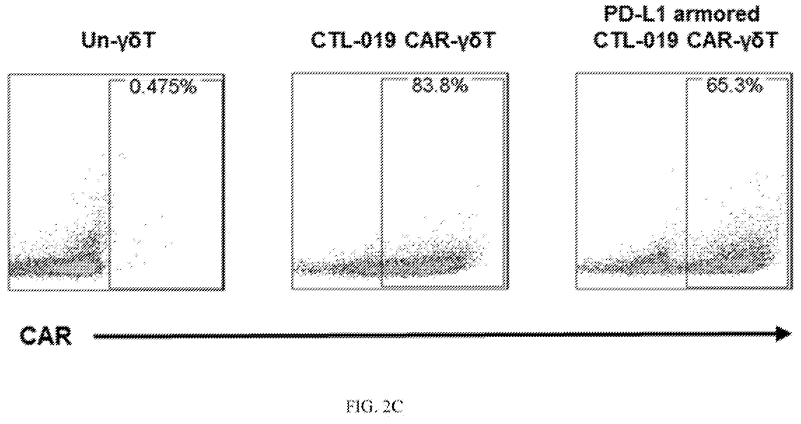

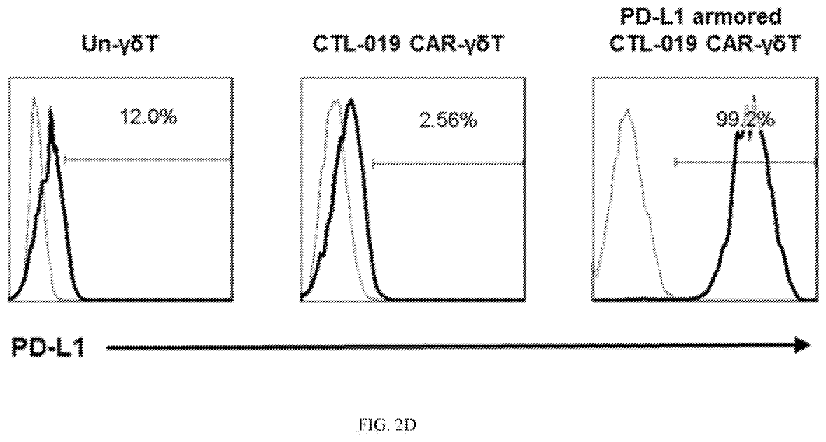

The results are shown in FIG. 2. Both anti-BCMA and anti-CD19 CARs can be expressed in αβ or γδ T cells at sufficient levels with >30% CAR positivity rate (FIGS. 2A and 2C). In addition, PD-L1 armor expression were determined to be >95% for the armored constructs, increased from <2% when not armored (FIGS. 2B and 2D) among the CAR+ cells.

### Example 4: In Vitro Killing and Cytokine Release

### Cytotoxicity Assay of αβ T Cells

Cytotoxicity of CAR-T cells, as well as the corresponding control αβ T cells were determined in a 20 h co-culture assay. In the experiments, the effector cells were collected by centrifugation, then diluted to the desired concentrations with 1640 phenol-red free medium (Invitrogen) supplemented with 2% heat inactivated FBS (Invitrogen). RPM I-8226, a human B lymphocyte that expresses target antigen BCMA, was chosen as the target cell line for anti-BCMA CAR-T cells. Raji, a human B lymphocyte that expresses target antigen CD19, was chosen as the target cell line for anti-CD19 CAR-T cells. The effector cells and the corresponding target cells were co-cultured at different effector to target ratios (E:T=2:1, 1:1, 0.5:1, 0.25:1 and 0:1 for RPMI-8226 and 1:1, 0.5:1, 0.25:1 and 0:1 for Raji) at 37° C. for 20 h in a 96 well plate. Control wells contained assay buffer only (1640 phenol red-free medium plus 2% hiFBS), target cell only (T), effector cell only (E) and max release of target cell (1% solution of Triton-X 100) Each condition was repeated in triplicates, and the cytotoxicity of effector cells was detected by a lactate dehydrogenase (LDH) assay kit (Roche). After completion of the 20 h co-culture, the assay plate was centrifuged, and supernatants were transferred to a new 96-well plate. The supernatants were diluted with an equal volume of the LDH assay reagent according to the manufacture's manual. The assay plate was incubated for about 30 min at 15° C.-25° C. The absorbance of the plate was measured at 492 nm and 650 nm using Flexstation reader (Molecular Devices) to provide relative cytotoxicity for each condition. To further determine long-term persistence, the effector cells were subjected to multiple rounds of target cell stimulation to determine their anti-tumor toxicity levels in the context of repeated antigen stimulation.

### Cytotoxicity Assay of γδ T Cells

Cytotoxicity of γδ T cells transduced with the lentiviral vectors containing constructs of Example 1 was assessed seven days post-transduction. Specifically, transduced or non-transduced γδ T cells were incubated with BCMA or CD19 positive target cell line, RPMI-8226 or Raji, and the cytotoxic effect of γδ T cells were evaluated using an LDH assay kit (Roche) as described above. Similarly, to further determine long-term persistence, the effector cells were subjected to multiple rounds of target cell stimulation to determine their anti-tumor toxicity levels in the context of repeated antigen stimulation.

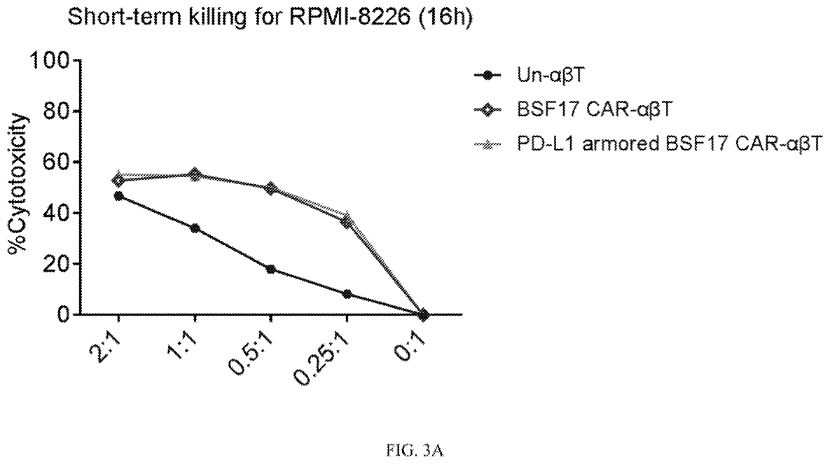

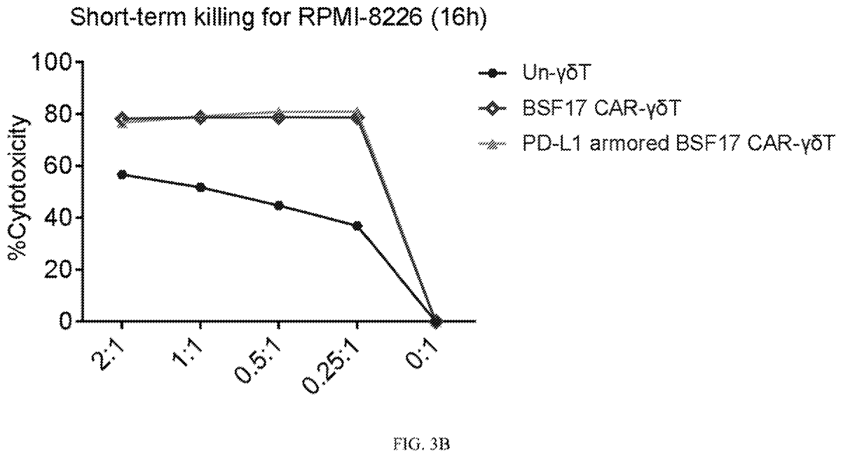

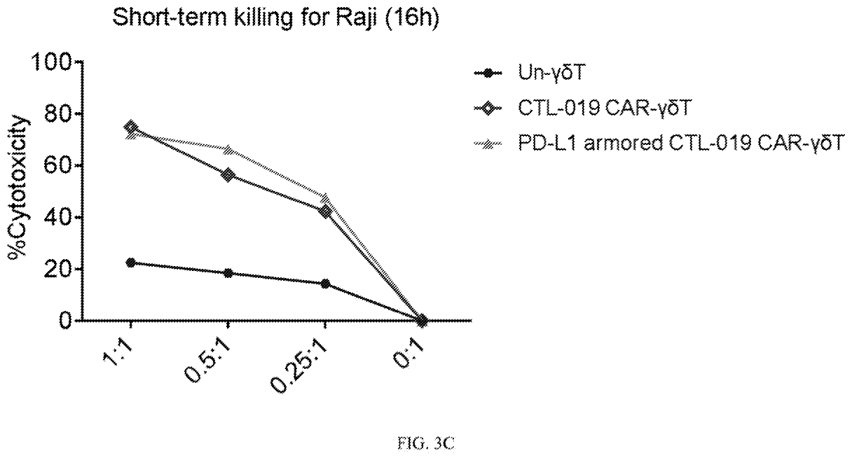

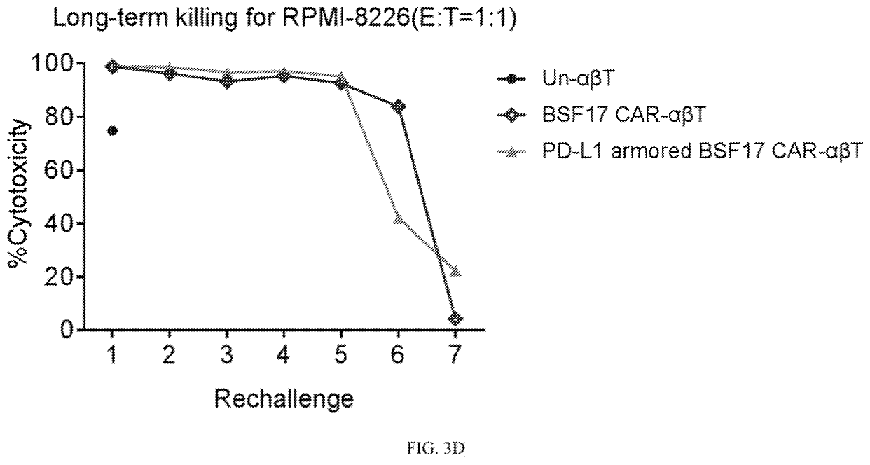

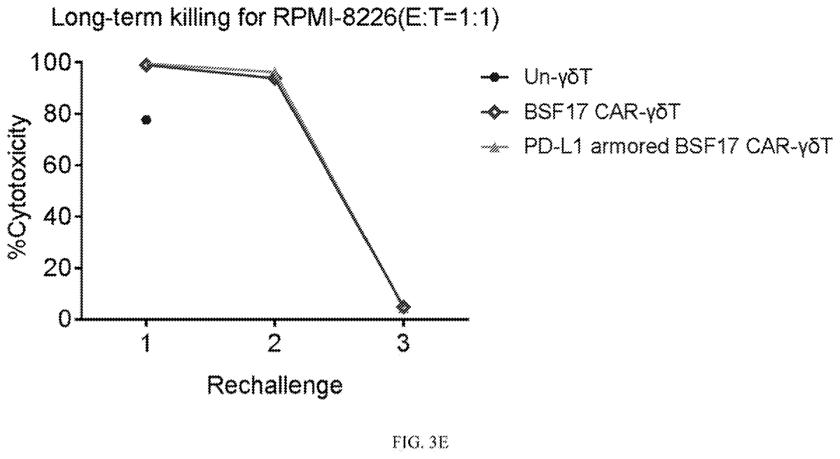

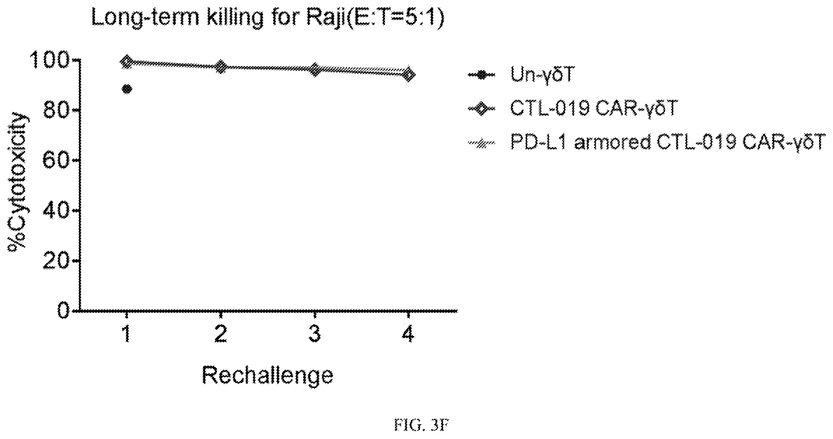

The results are shown in FIG. 3. We demonstrated that, for anti-BCMA designs, PD-L1 armored BSF17 CAR displayed similar level of cytotoxicity towards RPMI-8226 cells compared to unarmored BSF17 CAR in either αβ or γδ T cells at indicated E:T ratios (FIGS. 3A and 3B). Furthermore, for anti-CD19 designs, PD-L1 armored CRL-019 CAR displayed similar level of cytotoxicity towards Raji cells compared to unarmored CTL-019 CAR in γδ T cells at indicated E:T ratios (FIG. 3C). To further assess the long-terms persistence of these cells, repeated stimulation was conducted. We found that, for anti-BCMA designs, PD-L1 armored BSF17 CAR displayed similar level of long-term persistence towards RPMI-8226 cells compared to unarmored BSF17 CAR in either αβ or γδ T cells at indicated E:T ratios (FIGS. 3D and 3E). Furthermore, for anti-CD19 designs, PD-L1 armored CTL-019 CAR displayed similar level of long-term persistence towards Raji cells compared to unarmored CTL-019 CAR in γδ T cells at indicated E:T ratios (FIG. 3F). Taken together, these results suggest that the addition of a PD-L1 armor does not diminish CAR-T short-term cytotoxicity or alter its long-term persistence in vitro.

### Example 5: Cytokine Release

The supernatants of the cytotoxicity assay plate were collected for cytokine release analysis using commercial kits (Human IFN gamma kit, Cisbio, Cat #62HIFNGPEH; Human TNF alpha kit, Cisbio Cat #62HTNFAPEH). Other commercial kits can be used to assess release of additional cytokines: Human IL6 kit, Cisbio, Cat #62HIL06PEG; and Human IL2 kit, Cisbio, Cat #62HIL02PEH. The cell supernatants and an ELISA standard were dispensed directly into the assay plate for cytokine detection utilizing HTRF® reagents. The antibodies labeled with the HTRF donor and acceptor were pre-mixed and added in a single dispensing step.

The ELISA standard curve was generated using a Four-Parameter Logistic (4PL) curve. Regression using the standard curve enabled accurate determination of an unknown sample concentration across a wider range of concentrations than linear analysis, which was suitable for analysis of biological systems such as cytokine release. Applicable assay kits include Human IFN gamma kit (Cisbio, Cat #62HIFNGPEH) and Human TNF alpha kit (Cisbio, Cat #62HTNFAPEH).

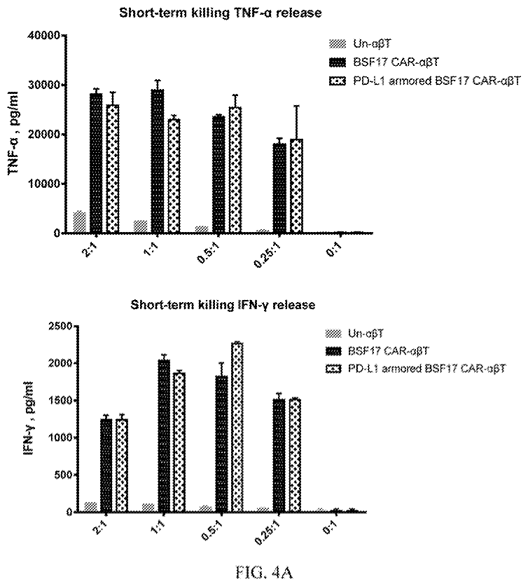

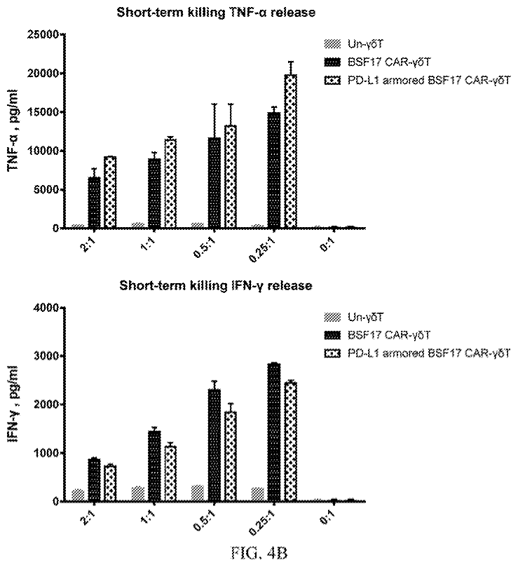

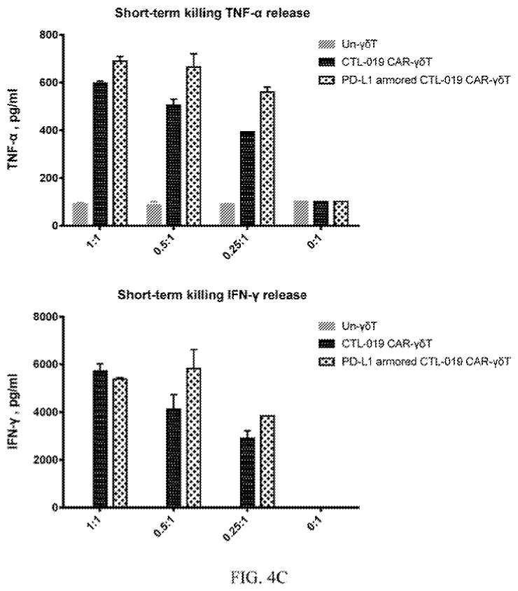

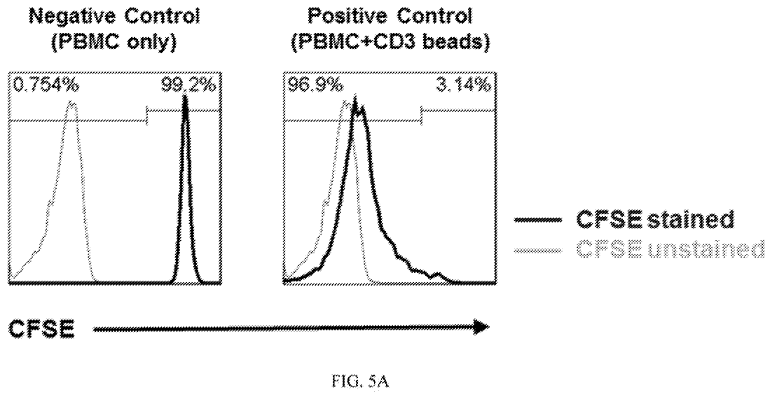

The results are shown in FIG. 4. We found that, for anti-BCMA designs, PD-L1 armored BSF17 CAR displayed similar level of TNF-α and IFN-γ cytokine production compared to unarmored BSF17 CAR in either αβ or γδ T cells at indicated E:T ratios (FIGS. 4A and 4B). Furthermore, for anti-CD19 designs, PD-L1 armored CTL-019 CAR displayed similar level of TNF-α and IFN-γ cytokine production compared to unarmored CTL-019 CAR in γδ T cells at indicated E:T ratios (FIG. 4C). Taken together, these results suggest that the addition of a PD-L1 armor does not alter CAR-T cytokine production profile in vitro.

### Example 6: In Vitro HvGD Assay

HvGD was assessed via a one-way mixed lymphocyte reaction (MLR) assay. Briefly, PD-L1 armored BSF17-CAR-γδ T cells (SEQ ID NO: 20), unarmored BSF17-CAR-γδ T cells (SEQ ID NO: 10), and untransduced γδ T cells were gamma-irradiated and co-incubated with CFSE-labelled HL A-mismatched allogeneic PBMCs from two different donors for a period of 7 days. At the end of the experiment, proliferation of allogeneic αβ T cells in each condition was evaluated by flow cytometry.

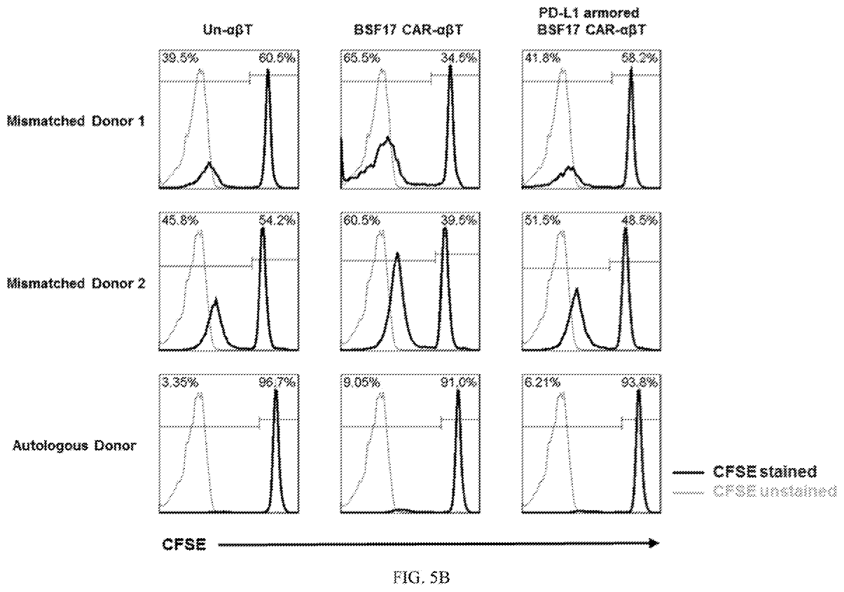

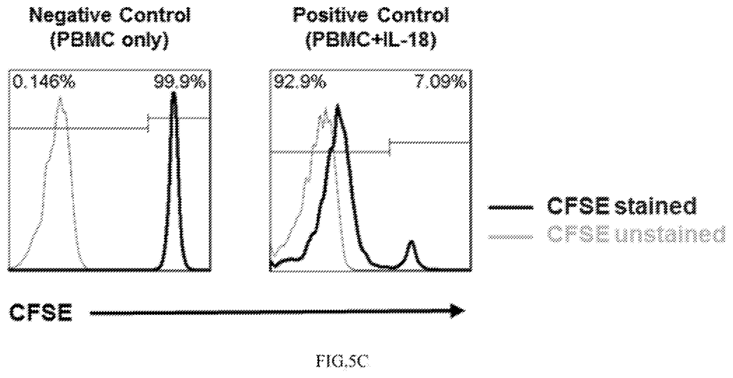

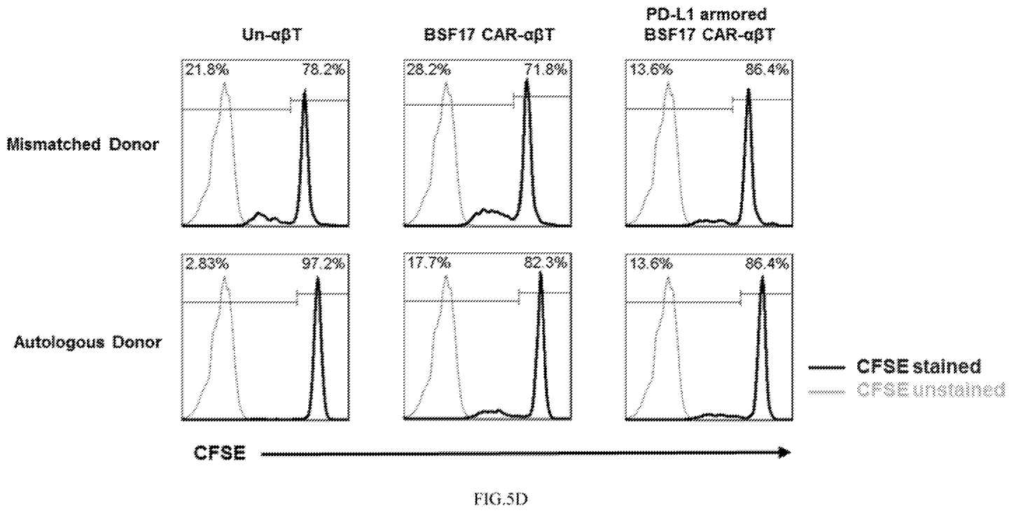

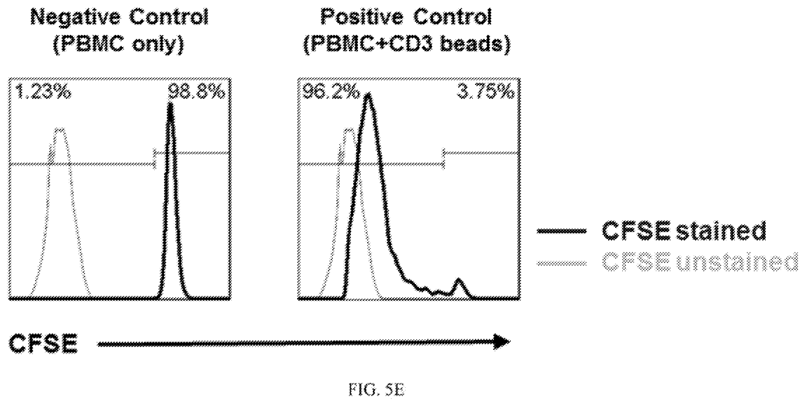

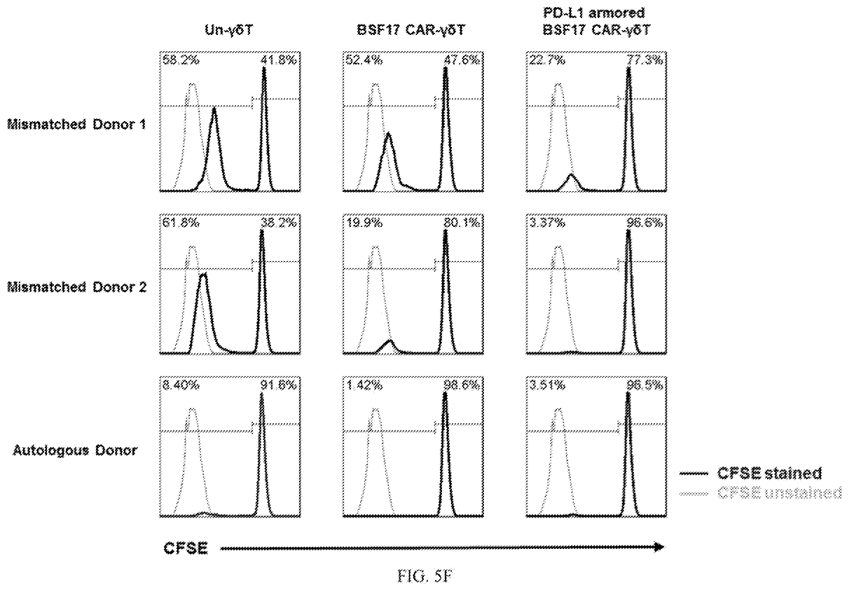

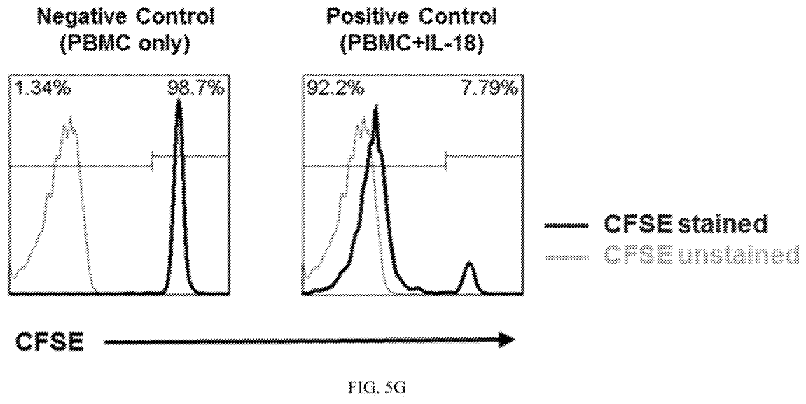

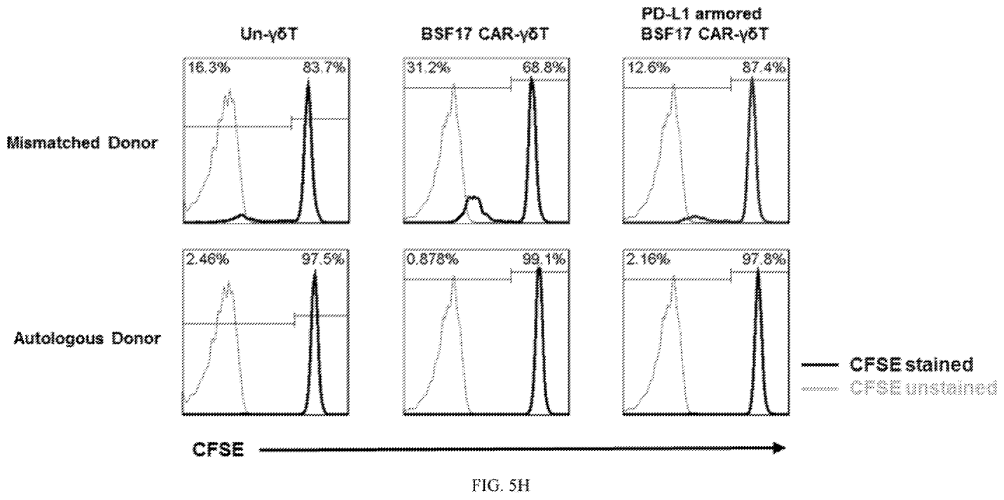

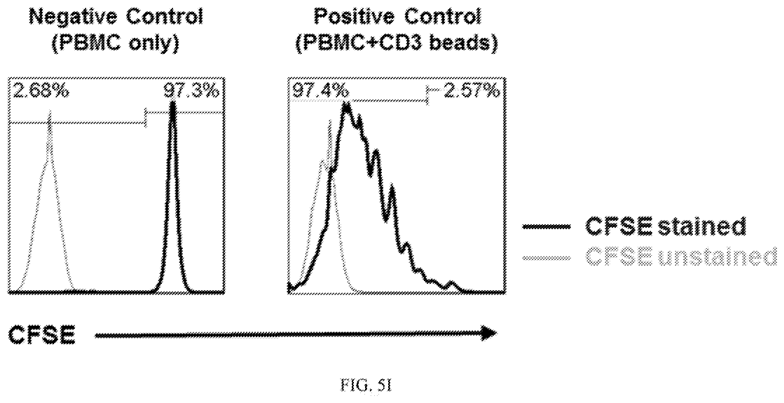

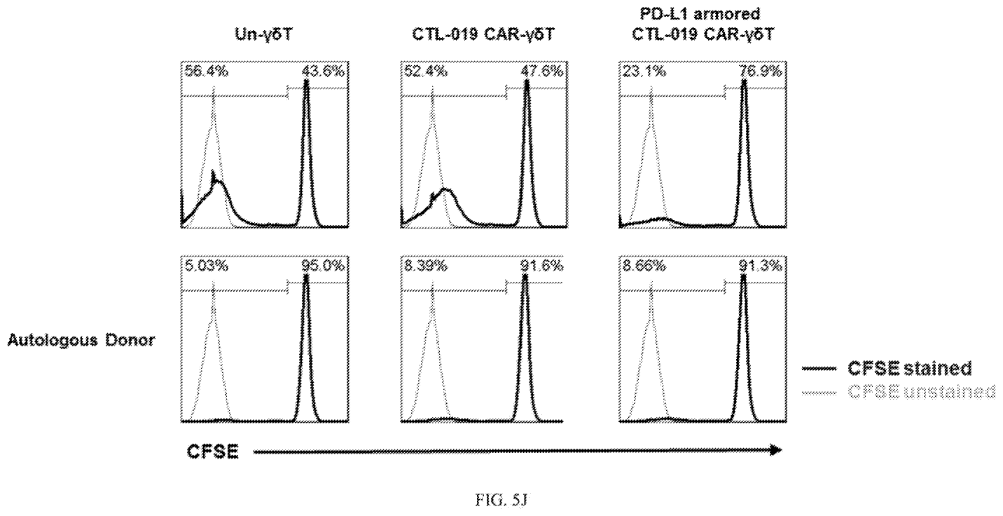

FIGS. 5A, 5C, 5E, 5G and 5I show results of the control groups, including PBMCs treated with CD3 beads (positive control for αβ T cells proliferation), PBMCs treated with IL-18 (positive control for NK cells proliferation) or PBMCs with no treatment which demonstrate robustness of the assay. To assess allogeneic αβ T cells proliferation in the PBMCs for example, in the right panel of FIG. 5A, 96.9% αβ T cells in PBMCs showed weak CFSE staining due to dilution of the CFSE dye by proliferated αβ T cells as a result of CD3 stimulation. In contrast, in the left panel of FIG. 5A, 99.2% of αβ T cells in PBMCs showed strong CFSE staining when PBMCs were untreated because little to no αβ T cell proliferation occurred in the absence of stimulation. Similarly, to assess allogeneic NK cells proliferation in the PBMCs, in the right panel of FIG. 5C, 92.9% NK cells in PBMCs showed weak CFSE staining due to dilution of the CFSE dye by proliferated αβ T cells as a result of IL-18, a well-established human NK cell stimulant, stimulation. In contrast, in the left panel of FIG. 5A, 99.9% of NK cells in PBMCs showed strong CFSE staining when PBMCs were untreated because little to no NK cell proliferation occurred in the absence of stimulation. Therefore, when allogeneic PBMCs were co-cultured with αβ or γδ T cells, higher HvGD corresponded to less strong CFSE staining as αβ or γδ T cells stimulated proliferation of αβ T or NK cells in the allogeneic PBMCs.

We first demonstrated the potential benefits of an ICL armor with an anti-BCMA, BSF17-CAR on αβ and γδ T cells. As shown in FIGS. 5B and 5F, for both PBMC donors, PD-L1 co-expression in the αβ or γδ CAR-T cells drastically reduced the proliferation of allogeneic αβ T cells. Specifically, in FIG. 5F, for mismatched Donor 1, more than 60% reduction of proliferation (from 52.4% to 22.7%) was observed when the γδ CAR-T cells were armored with PD-L1. For mismatched Donor 2, more than 80% reduction of proliferation (from 19.9% to 3.37%) was observed when γδ CAR-T cells were armored with PD-L1. Notably, for mismatched Donor 2, PD-L1 armored γδ T showed a minimal level of proliferation at 3.37%, which is comparable to that of the αβ T cells proliferation in autologous donor PBMCs (3.51%). In this particular case, HvGD was totally abolished in vitro. Similar, but to a lesser extent, effects were observed when αβ CAR-T cells were armored with PD-LL. Specifically, in FIG. 5B, for mismatched Donor 1, around 40% reduction of proliferation (from 65.5% to 41.8%) was observed when the αβ CAR-T cells were armored with PD-L1. For mismatched Donor 2, about 15% reduction of proliferation (from 60.5% to 51.5%) was observed when a0 CAR-T cells were armored with PD-L1. On the other hand, we also demonstrated that PD-L1 armor can reduce allogeneic NK proliferation in PBMCs. As shown in FIGS. 5D and 5H, around 50% reduction of proliferation (from 28.2% to 13.6%) was observed when the αβ CAR-T cells were armored with PD-L1. Meanwhile, approximately 60% reduction of proliferation (from 31.2% to 12.6%) was observed when γδ CAR-T cells were armored with PD-L1. Overall, these data demonstrates that a PD-L1 armor on γδ or αβ CAR-T cells can significantly reduce HvGD in vitro via the inhibition of proliferation of allogeneic αβ T and NK cells.

These findings were further supported by results of PD-L1 armored CTL-019 CAR on γδ T cells. We demonstrated, in FIG. 5J, that PD-L1 co-expression in the γδ CAR-T cells drastically reduced the proliferation of allogeneic αβ T cells. Specifically, about 60% reduction of proliferation (from 52.4% to 23.1%) was observed when the γδ CAR-T cells were armored with PD-L1. This results further expand the utility of this armor in other indications or molecule designs.

Taken together, we showed that the addition of ICL ligands, notably PD-L1, on CAR-αβ or γδ T cells could drastically reduce the risk of HvGD in vivo, thus highlighting its potential therapeutic use in allogeneic, off-the-shelf, cell therapy products.

### Example 7: In Vivo Anti-Tumor Efficacy of ICL-Armored CAR-T Cells

Anti-tumor activity of an exemplary anti-BCMA CAR-T was assessed in vivo in an RPMI-8226 (myeloma) xenograft model. Briefly, one million (1×10⁶) RPMI-8226 cells stably expressing the firefly luciferase reporter were implanted subcutaneously or intravenously on Day 0 in NOD/SCID IL-2RγCnull (NSG) mice. Fourteen days after tumor inoculation, mice were treated with intravenous injection of 1×10⁶ ICL-armored CAR-αβ or γδ T cells, untransduced control T cells, or phosphate-buffered saline (PBS). Tumor progression was monitored by bioluminescent imaging (BLI) once a week. In addition, T cell proliferation was monitored via FACS analysis of plasma samples of the treated mice. In addition, T cell proliferation was monitored via FACS analysis of plasma samples of treated mice.

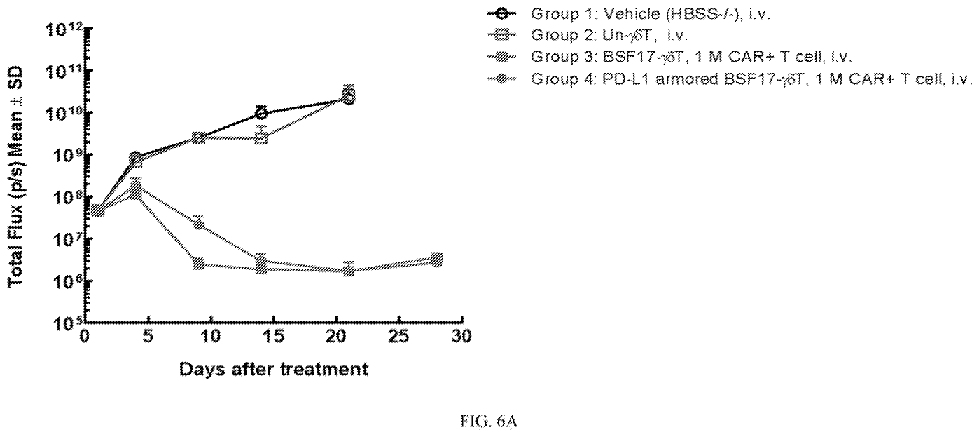

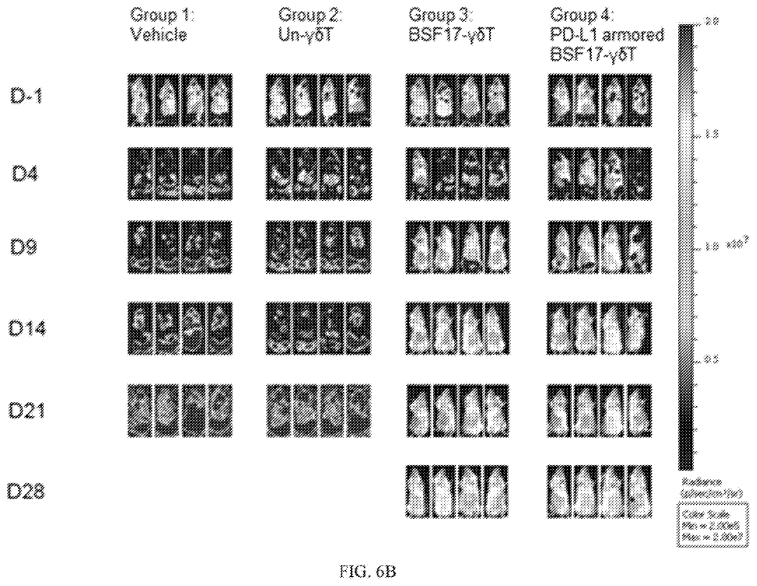

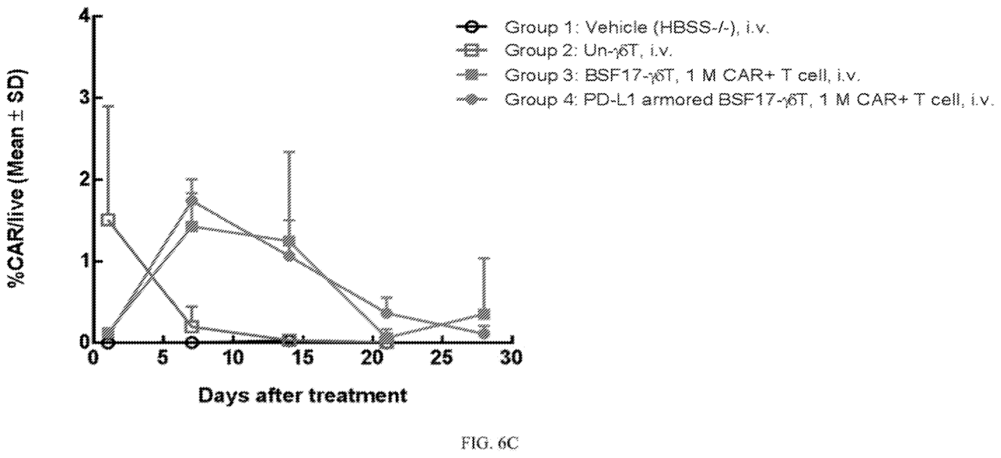

The results are shown in FIG. 6. We demonstrated that, for anti-BCMA designs, PD-L1 armored BSF17 CAR displayed similar level of in vivo efficacy compared to unarmored BSF17 CAR in γδ T cells (FIGS. 6A and 6B). Both designs showed superior anti-tumor cytotoxicity compared against control groups, as mice treated with either cells reached tumor-free status for at least two weeks while the control groups had to be sacrificed due to excessive tumor growth. Furthermore, PD-L1 armored BSF17 CAR displayed similar level of in vivo proliferation compared to unarmored BSF17 CAR in γδ T cells, with both groups peaked around 7 days post-treatment at around 1.5% CAR-T in peripheral blood (FIG. 6C). Taken together, these results suggest that the addition of a PD-L1 armor does not alter CAR-T anti-tumor efficacy or proliferation in vivo.

Furthermore, anti-tumor activity of an exemplary anti-CD19 CAR-T is assessed in vivo in a Raji (Burkitt's lymphoma) xenograft model. Briefly, one million (1-10⁶) Raji cells stably expressing the firefly luciferase reporter are implanted subcutaneously or intravenously on Day 0 in NOD/SCID IL-2RγCnull (NSG) mice. Seven days after tumor inoculation, mice are treated with intravenous injection of 1-10⁶ ICL-armored CAR-αβ or γδ T cells, untransduced control T cells, or phosphate-buffered saline (PBS). Tumor progression is monitored by bioluminescent imaging (BLI) once a week. In addition, T cell proliferation is monitored via FACS analysis of plasma samples of the treated mice.

### Example 8: In Vivo HvGD Against CAR-T Cells

HvGD is also evaluated in a mouse model, in which HLA-mismatched PBMCs are co-incubated with CAR-T cells in mice. Briefly, 2×10⁶ HLA-A2⁻ PBMCs are injected intravenously into NCG mice. Four days later, 5×10⁶ HLA-A2⁺ CAR-T cells, either ICL-armored or unarmored, or untransduced control T cells, are injected intravenously in the mice. Flow cytometry and blood routine assays are conducted to determine the severity of HvGD in each group of mice.

1. A modified therapeutic cell comprising a first heterologous nucleic acid sequence encoding an immune checkpoint ligand (ICL), wherein the therapeutic cell expresses a Major Histocompatibility Complex (MHC) molecule.

2-3. (canceled)

4. The modified therapeutic cell of claim 1, wherein the β2-microglobulin (B2M) gene of the modified therapeutic cell is not genetically modified.

5. (canceled)

6. The modified therapeutic cell of claim 1, wherein the ICL is selected from the group consisting of PD-L1, CD155, CD112, FGL1, galectin-9, CD47, B7H3, and B7H4.

7. (canceled)

8. The modified therapeutic cell of claim 1, wherein the ICL comprises an amino acid sequence selected from the group consisting of SEQ ID NOs: 1-8, or a variant thereof comprising an amino acid sequence having at least about 85% sequence identity to an amino acid sequence selected from the group consisting of SEQ ID NOs: 1-8.

9. The modified therapeutic cell of claim 1, wherein the modified therapeutic cell is an immune cell.

10. The modified therapeutic cell of claim 9, wherein the immune cell is selected from the group consisting of cytotoxic T cell, helper T cell, NK cell, NK-T cell, αβ T cell, γδ T cell, tumor-infiltrating T cell, dendritic cell (DC)-activated T cell, and peripheral blood mononuclear cell (PBMC).

11-18. (canceled)

19. The modified therapeutic cell of claim 1, further comprising a second heterologous nucleic acid sequence encoding an engineered receptor.

20. The modified therapeutic cell of claim 19, wherein the engineered receptor is selected from the group consisting of a chimeric antigen receptor (CAR), a recombinant T cell receptor (TCR), a T-cell antigen coupler (TAC) receptor, and a TCR fusion protein (TFP).

21-26. (canceled)

27. The modified therapeutic cell of claim 19, wherein the first heterologous nucleic acid sequence and the second heterologous nucleic acid sequence are present in a vector.

28. The modified therapeutic cell of claim 27, wherein the first heterologous nucleic acid sequence is fused to the second heterologous nucleic acid sequence via a third nucleic acid sequence encoding a self-cleavable linker.

29. The modified therapeutic cell of claim 28, wherein the vector comprises a nucleic acid sequence encoding an amino acid sequence selected from the group consisting of SEQ ID NOs: 12-27.

30. (canceled)

31. A pharmaceutical composition comprising the modified therapeutic cell of claim 1.

32. A method of treating a disease or condition in an individual in need thereof, comprising administering to the individual an effective amount of the pharmaceutical composition of claim 31.

33-34. (canceled)

35. The method of claim 3, wherein one or more human leukocyte antigen (HLA) alleles of the individual have mismatching allotypes compared to those of the modified therapeutic cell.

36. The method of claim 35, wherein the one or more HLA alleles are selected from the group consisting of HLA-A, HLA-B, HLA-C, and HLA-DRB1.

37. The method of claim 3, wherein all tested HLA alleles of the individual have matching allotypes compared to those of the modified therapeutic cell.

38. The method of claim 32, wherein the method:
i) reduces undesired immune response against the modified therapeutic cell in the individual compared to a method using a therapeutic cell that does not comprise the first heterologous nucleic acid sequence encoding the ICL; and/or
ii) induces immune tolerance towards the modified therapeutic cell in the individual compared to a method using a therapeutic cell that does not comprise the first heterologous nucleic acid sequence encoding the ICL.

39. The method of claim 38, wherein the undesired immune response comprises Host-versus-Graft (HvG) response.

40. (canceled)

41. The method of claim 32, wherein the disease or condition is a cancer, an infectious disease, or an autoimmune disease.

42. A method of reducing graft rejection of allogeneic therapeutic cells in an individual in need thereof, comprising administering to the individual an effective amount of the allogeneic therapeutic cells, wherein the allogeneic therapeutic cells comprise a first heterologous nucleic acid sequence encoding an ICL, and wherein the allogeneic therapeutic cells express an MHC molecule.

43. (canceled)
